(19) United States
(12) Patent Application Publication
(10) Pub. No.: US 2024/0389989 A1
(43) Pub. Date: Nov. 28, 2024

(54) BIOPSY SITE TISSUE REPAIR DEVICE AND METHODS

(71) Applicant: Joseph Choate BURKETT, Addison, TX (US)

(72) Inventor: Joseph Choate BURKETT, Addison, TX (US)

(21) Appl. No.: 18/646,333

(22) Filed: Apr. 25, 2024

Related U.S. Application Data
(63) Continuation of application No. PCT/US2022/078919, filed on Oct. 28, 2022.
(60) Provisional application No. 63/272,997, filed on Oct. 28, 2021.
(60) Provisional application No. 63/319,195, filed on Mar. 11, 2022.
(60) Provisional application No. 63/397,250, filed on Aug. 11, 2022.
(60) Provisional application No. 63/413,161, filed on Oct. 4, 2022.

(51) Int. Cl. A61B 17/00; A61B 10/02

(52) U.S. Cl. CPC A61B 17/0057; A61B 10/0275; A61B 2010/0208; A61B 2017/00389; A61B 2017/00477; A61B 2017/00575; A61B 2017/00623; A61B 2017/0065; A61B 2017/00831

(57) ABSTRACT

An apparatus for performing a biopsy and preventing hemorrhaging at the site of the biopsy is disclosed herein. Methods of preventing hemorrhaging at the site of a biopsy by sealing or repairing damaged tissue are also disclosed.

### CROSS-REFERENCE

This application is a continuation of International Application No. PCT/US2022/078919, filed Oct. 28, 2022, which claims benefit of U.S. Provisional Application No. 63/272,997, filed Oct. 28, 2021, U.S. Provisional Application No. 63/319,195, filed Mar. 11, 2022, U.S. Provisional Application No. 63/397,250, filed Aug. 11, 2022, and U.S. Provisional Application No. 63/413,161, filed Oct. 4, 2022, all of which are herein incorporated by reference in their entireties.

### BACKGROUND

Despite previous attempts at improving the safety of biopsy, endoscopy, and various other surgical procedures, current technology and prior art fall short of effectively preventing hemorrhage and other major complications. Many of these procedures are routine in nature but pose serious risk, even to otherwise healthy individuals.

Furthermore, even when these procedures may provide life-saving, treatment guiding information, the risk for complications prevents their use. Risk factors for these problems can be intrinsic (such as comorbid disease), extrinsic (such as anticoagulant medications), or both, and are often compounding in nature. In kidney disease, for example, etiologies of disease frequently overlap or rapidly progress. This requires clinicians perform kidney biopsy procedures to obtain diagnostic and treatment-guiding information. A large percentage of these patients, however, have dysfunctional coagulation systems, elevated blood pressures, diseased friable organ tissue, and suffer from concomitant cardiac disease or vascular fibrosis. These risk factors restrict kidney biopsy procedures to a fraction of the indicated use, prevents patients from getting the resulting treatment benefits, and has a high cost of morbidity and mortality. The same can be said for liver disease, neoplastic disease, neurological disease, gastrointestinal disease, among many others.

Patients with mild to moderate disease are still at considerable risk for these complications. As many as ten percent of liver or kidney biopsy patients experience major hemorrhage, requiring blood transfusions and prolonged hospital stay. The risk after kidney biopsy is so high, even relatively young patients without any comorbidities are kept for observation in upwards of 50% of cases. Many other types of needle or biopsy-based procedures safe have unacceptable rates of complication as well. Prostate biopsies result in residual rectal bleeding in 33% of cases, brain biopsies result in intercranial hemorrhage in 5-10% of cases, and gastrointestinal/pancreatic/and thoracic procedures all have high inherent risk of complications.

In kidney biopsy, all 3 components of the coagulation system are disrupted during the course of disease progression. A high tensile strength plug does not prevent lateral bleeding, especially in severely hypertensive individuals. Plugging without sealing also sets these kidney and liver disease patients up for higher risk of delayed bleeding. The plug may hold for some time period, but high internal and external adhesive forces are a better measure for preventing propagation of internal patch cracks or damage, as well as cracks that might otherwise propagate along the interface seal between the patch materials and the tissue.

The device described in this application solves numerous, previously unrecognized physiological and pathophysiological issues that predispose organs to bleeding or other complications during instrumentation. Embodiments of the device presented in this paper use one or more measures to rapidly or immediately seal some or all of damaged edges biological tissues, organs, vessels, luminous structures, and substances.

### SUMMARY

In some embodiments, this device is designed to rapidly seal biological tissue, structures, organs, vessels, and other biological structures. In some embodiments, the device is designed to perform biopsy sampling procedures with simultaneous or sequential sealing the damaged tissue. In some embodiments, this is accomplished by using one or more measures, including patch material, thermal cautery, RF electrosurgery, acoustic ultrasonic cautery, freezing, plugging, or other methods described herein.

In some embodiments, the patch material is a photopolymerized substance. In some embodiments these photopolymerized patch materials are created from a dual network of molecules, including more than one type of monomers, mixtures, or substances. In some embodiments, the device is designed to rapidly activate highly adhesive photopolymerizing patch material. In some embodiments, the patch material does not rely of photopolymerization to seal biological substances.

In some embodiments, the RF electrosurgery used to seal the tissue is monopolar in nature, bipolar in nature, several types. In some types of embodiments, there is a combination of patch materials used, before, during, or after the use of one other the other tissue altering methods. In some embodiments, the device comprises the use of a combination is RF electrosurgery along with deploying a photoactivated patch material. In some embodiments, the device uses one or more synthetic patch materials, natural patch materials, patch materials not requiring photoactivation, other patch materials disclosed herein, or some combination thereof. In some embodiments, acoustic cautery or thermal cautery is used in combination with deploying a patch material. In some embodiments acoustic cautery or thermal cautery is used in combination with deploying and activating a photopolymerizable patch material.

In some embodiments, the device emits photons from one or more the functioning end to activate photo-sensitive substances being utilized as a biocompatible patch material. In some embodiments, this can be used for one or more of the following: sealing wounds, sealing tissues, sealing vessels, sealing tubular structures, preventing hemorrhage, coagulating blood, preventing fluid release, repairing defects, creating tubular bodies, repairing tubular bodies, modifying the lumen of tubular bodies, or other suitable uses and combinations thereof. In some embodiments, the device emits the photons from one or more of the following portions of the device: through the needle body, shaft, interior, exterior, tip, and/or other portions, the cutting end and/or body, and through the interior and/or exterior surfaces.

In some embodiments of the device there is a device housing. In some embodiments herein, the proximal side of the device refers to the portion of the device away from the tip used on a patient, animal, or target tissue. In some embodiments herein, the distal side of the device refers to the portion of the tip or portion device away from the device used at a biological site on a patient, animal, or target tissue. In some embodiments of the device, there the biocompatible patch material comprising photoactivated (or photoinitiated) substance (or mixture) is expelled from a distal portion of the device to a biopsy sample void, biopsy tract, biopsy area, needle tract, instrumented tissue, injured area, lacerated area, wound, lactation, surgical site, vessel, nerve, damaged tissue, central nervous system tract, kidney tissue, liver tissue, prostate tissue, brain tissue, breast tissue, tumor tissue, mass tissue, lung, heart, blunt trauma area, abrasion area, or some other biological tissue area. This site may be referred to as the biological site, tissue repair site, or biological repair site herein. In some embodiments, the substance prevents bleeding, vascular damage, vascular malformation, extrusion of saliva, extrusion of bile, extrusion of fecal matter, extrusion of cerebrospinal fluid, extrusion of urine, or some other biological substance. In some embodiments, the substance is applied on the one or more of the following locations: on medical instrument, adjacent to the medical instrument, some distance from the medical instrument, biopsy location, biopsy tract, instrumentation tract. In some embodiments, photoinitiation of the biocompatible patch material is conducted by photons emitted from one or more of the following locations: needle tip, the shaft of the needle, a catheter, a sheath, a waveguide, a light diffuser, a light projector, a lamp, a laser, housing of the device, a biopsy portion of the device, a mobile portion of the device, a static portion of the device, some other aspect of the device, or a combination thereof.

In some embodiments, the device is intended for use with one or more types of the following biopsy procedures: fine needle aspiration biopsy, core needle biopsy, aspiration biopsy, needle biopsy, cryo-biopsy, vibrating needle biopsy, biopsy guns, spring loaded biopsy, actuator activated biopsy, pneumatic controlled biopsy, biopsy using heat, biopsy using electrocautery, biopsy using freezing, automatic biopsy, hydraulic biopsy, multiple needle biopsy, multiple pass biopsies, among others. In some embodiments, the device is intended for use during one or more of the following procedures: surgical procedures, endoscopic procedures, colonoscopic procedures, bronchoscopic procedures, vascular procedures, angioplasty procedures, biopsy procedures, arterial procedures, sinuplasty procedures, graft procedures, fistula procedures, neurosurgical procedures, cardiovascular procedures, urological procedures, nephrology procedures, breast procedures, lung procedures, gastrointestinal procedures.

In some embodiments, the device is intended for use with one or more organs, tissues, biological liquids, biological structures, biological substances, and/or other biological materials. In some embodiment, one or more of organs, structures, and/or materials is all and/or a portion of the head, the brain, the subarachnoid space, the outer ear, the inner ear, the middle ear, the sinus cavities, the face, the nasal cavities, oral space, the oropharyngeal space, in or the neck, the spine, the chest, the lung, the heart, the trachea, the bronchioles, the lungs, the abdomen, the kidney, the liver, a vessel, a nerve, an artery, the aorta, the esophagus, the stomach, the small bowel, the bladder, the large bowel, a bone, the central nervous system, the cornea, the eye, the biliary tract, a fistula, an ulcer, an acute wound, a tubular body, a chronic wound, the pancreas, the abdomen, the legs, the feet, the arms, the hands, the gastrointestinal the rectum, the skin, the nervous system, peripheral nerves, the hands, the feet, the lower legs, the fingers, the toes, the eyes, and/or some other portion of tissue, organ, cells, biological substance, or biological material.

In some embodiments, the device is intended for performing, assisting, treating, covering, sealing, sealing the tract of, aiding the repair of, or a combination thereof, of the one or more of the following procedures: a core needle biopsy, a fine needle biopsy, insertion of a needle, arterial wound, arterial hemostasis, surgical hemostasis, ligation of a vessel, ligation of an artery, ligation of a fallopian tube, lumbar puncture, CNS instrumentation, eye surgery, ear surgery, ENT surgery, Tonsillectomy, sinus surgery, nerve surgery, ligation tissue, treatment for a surgical wound, treatment for a traumatic wound, tr treating a mass, stenting an artery, stenting a vein, stenting a lymphatic duct, stenting an cerebral spinal fluid channel, repairing stenting a bile duct, stenting a pancreatic duct, stenting an injury to a tubular structure, stenting a vessel with damage related to thermal or radiation damage or injury, stenting a vessel tubular structure that has malignant infiltration, stenting the aorta, stenting an artery affected by peripheral artery sclerosis, stenting a cerebral vessel, stenting in an area of previous stenosis, stenting in the area of a previous stent, stenting a vessel or tubular structure damaged by penetrating trauma, stenting a vessel or tubular structure damaged by blunt force trauma, and/or a vessel or tubular structure damaged by explosive or pneumatic injury.

In some embodiments, the device is used for biopsy, such that the needle portion of the device conducts, transmits, and or/generates photons, such that those photons are transmitted to a photoactivated substance which causes one or more of the following actions: seal tissue, seal cells, seal tubular structures, seal vessels, seal arteries, solidify, liquify, harden, soften, turn into gel, become more viscous, become less viscous, become more thicker, become thinner, become more elastic, become less elastic, become more rigid, become less rigid, become more flexible, become less flexible, become more pliable, become less pliable, become more adherent, to become less adherent, to maintain shape, to release shape, to relax shape, to solidify surrounding structures, to weaken surround structures, to fix relative and/or absolute position of one or more surround structures, to fix relative or and/or absolute position structures within its internal and/or inner lumen.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-IG depict a device for collecting a tissue sample and repairing tissue at a biopsy site, according to some embodiments;

FIGS. 2A-2D depict various components of tissue repair device, according to some embodiments;

FIGS. 3A-3E depict a waveguide for transmitting light through a lumen of a biopsy needle, according to some embodiments;

FIGS. 4A-4E depict an extendible light rod for curing a photopolymerizable biocompatible patch material dispensed at a tissue repair site, according to some embodiments;

FIGS. 5A-5D depict rotating sheath for acquiring a tissue sample during a biopsy, according to some embodiments;

FIGS. 6A-6D a catheter deliverable device for collecting tissue and repairing tissue at a biopsy site, according to some embodiments;

FIGS. 7A-7D depict a device for repairing tissue, according to some embodiments;

FIGS. 8A-8E depict a device for repairing tissue, according to some embodiments;

FIGS. 9A-9C depict a needle cartridge component of a device for repairing tissue, according to some embodiments;

FIGS. 10A-10E depict a needle cartridge component of a device for repairing tissue, according to some embodiments;

FIGS. 11A-11D depict a device for repairing tissue, according to some embodiments;

FIGS. 12A-12F depict a device for repairing tissue, according to some embodiments;

FIGS. 13A-13E depict a needle cartridge component of a device for repairing tissue, according to some embodiments;

FIGS. 14A-14L depict components of a cocking mechanism of a device for repairing tissue, according to some embodiments;

FIGS. 15A-15F depict components of a cocking mechanism of a device for repairing tissue, according to some embodiments;

FIGS. 16A-16D depict components of a device for repairing tissue, according to some embodiments;

FIGS. 17A-17B depict a removeable light source of a device for repairing tissue, according to some embodiments;

FIGS. 17C-17D depict a recess for receiving a removeable light source of a device for repairing tissue, according to some embodiments;

FIGS. 18A-18E depict a needle cartridge component of a device for repairing tissue, according to some embodiments;

FIGS. 19A-19D depict a needle cartridge component of a device for repairing tissue, according to some embodiments;

FIGS. 20A-20D depict a needle cartridge component of a device for repairing tissue during delivery of a biocompatible patch material, according to some embodiments;

FIGS. 21A-21D depict a device for repairing tissue, according to some embodiments;

FIGS. 22A-22D depict one or more fluid manipulation components of a device for repairing tissue, according to some embodiments;

FIGS. 23A-23D depict one or more fluid manipulation components of a device for repairing tissue, according to some embodiments;

FIGS. 24A-24E depict one or more fluid manipulation components of a device for repairing tissue, according to some embodiments;

FIGS. 25A-25E depict one or more fluid manipulation components of a device for repairing tissue, according to some embodiments;

FIGS. 26A-26C depict one or more fluid manipulation components of a device for repairing tissue, according to some embodiments;

FIGS. 27A-27C depict one or more fluid manipulation components of a device for repairing tissue, according to some embodiments;

FIG. 28A depicts one or more fluid manipulation components of a device for repairing tissue, according to some embodiments;

FIG. 28B depicts a method for using a device for repairing tissue, according to some embodiments;

FIG. 29 depicts one or more fluid manipulation components of a device for repairing tissue, according to some embodiments;

FIG. 30 depicts one or more fluid manipulation components of a device for repairing tissue, according to some embodiments;

FIG. 31 depicts one or more fluid manipulation components of a device for repairing tissue, according to some embodiments;

FIG. 32 depicts one or more fluid manipulation components of a device for repairing tissue, according to some embodiments;

FIG. 33 depicts one or more fluid manipulation components of a device for repairing tissue, according to some embodiments;

FIG. 34 depicts one or more fluid manipulation components of a device for repairing tissue, according to some embodiments;

FIG. 35 depicts an automated needle retraction component of a device for repairing tissue, according to some embodiments;

FIGS. 36A-36B depict a needle retraction component of a device for repairing tissue, according to some embodiments;

FIGS. 37A-37B depict an automated light source toggle component of a device for repairing tissue, during delivery of a biocompatible patch material, according to some embodiments;

FIG. 38 depicts a sterile sleeve for containing at least a portion of a device for repairing tissue utilizing cauterization, according to some embodiments;

FIG. 39A-39D components of a tissue repair device, according to some embodiments;

FIGS. 40A to 40F depict a method of using a tissue repair device comprising a balloon to provide compression during tissue repair, according to some embodiments;

FIGS. 41A to 41F depict a method of using a tissue repair device comprising an extendable light source to repair a biopsy site, according to some embodiments;

FIGS. 42A to 42E depict a method of partially photoactivating a biocompatible patch material using a tissue repair device comprising an extendable light source, according to some embodiments;

FIGS. 43A to 43F depict a method of using a tissue repair device comprising a light emitting catheter to repair a biopsy site, according to some embodiments;

FIGS. 44A to 44F depict a method of using a tissue repair device comprising a light emitting catheter to repair a biopsy site by depositing a material anchor, according to some embodiments;

FIGS. 45A to 45E depict a method of using a tissue repair device to repair a biopsy site with cauterization, according to some embodiments;

FIGS. 46A to 46E depict a method of using a tissue repair device to repair a biopsy site with cauterization and delivery of a biocompatible patch material, according to some embodiments; and

FIGS. 47A-47B depict a plate which ultrasound waves can be transmitted through, according to some embodiments.

### DETAILED DESCRIPTION

Provided herein are embodiments of a device for repairing tissue by delivering a biocompatible patch material to a target site. In some embodiments, the biocompatible patch material comprises is activated to cause a phase change or change in viscosity. In some embodiments, the patch material is photoactivated. In some embodiments, the patch material is not photoactivated, but is still capable of stopping bleeding from arterial level hydrostatic pressure bleeding. In some embodiments, the device is able to stop arterial pressure bleeding with a combination of tissue altering mechanism along with delivery of patch material.

### I. Biocompatible Patch

In some embodiments, the device delivers and activates biocompatible patch material to one or more of an organ, tissue, vessel, artery, cavity, vein, nerve, lumen, skin, mucosa, tube, lymphatic tissue, muscle, bone, biological surface, biological interface, tubular body, other biological substances, or some combination thereof. In some embodiments, the device comprises the components, substances, and controls to deliver and activate that patch material to one or more biological locations. In some embodiments, the biological location has undergone instrumentation related to one or more of a biopsy site, biopsy wound, biopsy tract, needle tract, surgical tract, vessel ligation, arterial ligation, instrumented tissue, vascular procedure, endoscopic procedure, hemostatic procedure, bronchoscopy procedure, gastrointestinal procedure, wound treatment, traumatic wound, ulceration, fistula treatment, surgical wound treatment, acute wound treatment, chronic wound treatment, other surgical procedure, or a combination thereof. In some embodiments, the device delivers and seals the patch material to these areas in conjunction with one or more procedures, including, but not limited to: core needle biopsy, fine needle biopsy, fine needle aspiration, vacuum assisted biopsy, single handed biopsy, RF electrosurgery, hemostatic procedure, tissue sealing procedure, or some combination thereof.

In some embodiments, the device can be used to effectively cut and remove a biopsy sample from an internal organ, such as the liver or kidney, while also able to deliver and cure a biocompatible photopolymerizing patch material areas of tissues and biological substances altered by the instrumentation. In some embodiments, the patch material is activated by photopolymerization, and generates adhesive forces (internal, external, or both) exceeding that of hypertensive human hydrostatic blood pressures. In some embodiments, the device and patch material are designed to limit the curing, action, and/or function of the sealed patch material to one or more target locations.

In some embodiments, the device may perform one or more of those functions, while also able to perform another tissue altering procedure, such as RF electrosurgery, ultrasound (US) vibrational cautery, thermal cautery, some other tissue altering procedure, or some combination thereof as described herein.

In some embodiments, the photoactivated patch material withstands hydrostatic forces of about 120 mmHg to about 300 mmHg. In some embodiments, the photoactivated patch material withstands hydrostatic forces of about 120 mmHg to about 130 mmHg, about 120 mmHg to about 140 mmHg, about 120 mmHg to about 200 mmHg, about 120 mmHg to about 250 mmHg, about 120 mmHg to about 300 mmHg, about 130 mmHg to about 140 mmHg, about 130 mmHg to about 200 mmHg, about 130 mmHg to about 250 mmHg, about 130 mmHg to about 300 mmHg, about 140 mmHg to about 200 mmHg, about 140 mmHg to about 250 mmHg, about 140 mmHg to about 300 mmHg, about 200 mmHg to about 250 mmHg, about 200 mmHg to about 300 mmHg, or about 250 mmHg to about 300 mmHg. In some embodiments, the photoactivated patch material withstands hydrostatic forces of about 120 mmHg, about 130 mmHg, about 140 mmHg, about 200 mmHg, about 250 mmHg, or about 300 mmHg. In some embodiments, the photoactivated patch material withstands hydrostatic forces of at least about 120 mmHg, about 130 mmHg, about 140 mmHg, about 200 mmHg, or about 250 mmHg.

In some embodiments, the patch material is activated all or in part while the patch material is within some portion of the device. This preliminary activation can take place within the bore of the needle, within the housing of the device, within the lumen of a catheter, within the patch cartridges, within the needle cartridge, within the handle of a ligation device, within some other location before initiation at the wound site, or some combination thereof.

In some embodiments, the patch material is specifically formulated, delivered, and cured to enable it to seal tissue and substances disrupted by biopsy procedures such that it prevents significant bleeding from disrupted vessels or structures which have an internal hydrostatic force of arterial blood pressures (120 mmHG) or higher. In some embodiments, these patch materials are formulated, delivered, and cured so they can seal disrupted vessels, tissues, fluids, turbulent fluid motion, bleeding, vascular malformation structures with an internal luminal pressure, constant, pulsatile, hydrostatic force, or some combination thereof, at pressures equivalent to elevated human blood pressures (e.g., 140, 150, 160, . . . 300 mmHg) or higher.

In some embodiments, the device delivers and/or cures the patch material through a needle or small lumen component, with an internal diameter of a 16 gauge needle (1.194 mm) or smaller lumen size. In some embodiments, the device is able to alter the tissue or substances at or near the target location to improve the delivery and/or sealing related properties of the patch material (e.g., cauterizing the tissue in preparation for adhesion to the patch material).

In some embodiments, the patch material delivered and activated by this device is one or more types of photopolymerized patch material. In some embodiments, the photopolymerized patch material is used to seal certain parts or entire areas related to the related procedural instrumentation such as altered or damaged tissues, tracts, vessels, lumen containing bodies, nerves, organs, capsules, structures, anatomical anomalies, and other biological substance, or some combination thereof. Delivery and activation of these photopolymerized patch materials can be temporally adjacent, temporally separated, selected, semi-automatic, automatic, or a combination thereof. In some embodiments, the properties of the photopolymerized patch material are altered or activated before, during, or after the normal actions of the procedure are performed, such as just after cutting a biopsy sample from a tissue bed. Delivery of a photoactivated patch material can be coupled with tissue alteration by ultrasonic vibrational cautery, RF electrosurgery (monopolar or bipolar), resistive cautery, other method of cautery, freezing, or combinations thereof.

In some embodiments, photoactive patch materials are delivered during the procedure to the specific target location during the procedure itself, without significantly altering the user's procedural steps. In some embodiments, components are be mixed at or near the distal aperture from which they are emitted, such that they travel through the device with a reduce viscosity relative to the viscosity after mixing. In some embodiments, substances are heated or cooled to reduce the viscosity. In some embodiments, the patch material includes one or more photo-initiators, monomers, dimers, oligomers, polymers, synthetic substances, natural substances, semi-synthetic substances, salutes, solvents, nano-particles, additives, other substances, or a combination there of.

In some embodiments, the photo-initiator which is used activate or help activate the patch material is one or more of the following: LET (Lithium phenyl-2,4,6-trimethylbenzoylphosphinate), Irgacure Diphenyl(2,4,6-trimethylbenzoyl)phosphine oxide, Ruthenium (Ru) metal complex, Bipyridine, Ruthenium (Ru) metal complex, Eosin-Y, TEA, 1-vinyl-pyrrolidinone, Triethanolamine (TEA, co-initiator), N-Vinylcaprolactam (Co-initiator), Omnicure, 2,2-azobis [2-methyl-(2-hydroxyethyl) propionamide] (VA-086), or a combination there of.

In some embodiments, the viscosity or rheological values required of photopolymerized patch material are capable of stopping and preventing bleeding from arteries, other vessels, lumen containing bodies, tissues, or substances at hydrostatic forces of arterial levels requires a viscosity, toughness, and rheological values significantly higher than those previously described. In some embodiments, the viscosity of the biocompatible patch material upon delivery to the biological site is about 15 centipoise (cp) to about 100,000 cp. In some embodiments, the viscosity of the biocompatible patch material upon delivery to the biological site is about 15 cp to about 50 cp, about 15 cp to about 100 cp, about 15 cp to about 500 cp, about 15 cp to about 1,000 cp, about 15 cp to about 3,000 cp, about 15 cp to about 5,000 cp, about 15 cp to about 10,000 cp, about 15 cp to about 20,000 cp, about 15 cp to about 100,000 cp, about 50 cp to about 100 cp, about 50 cp to about 500 cp, about 50 cp to about 1,000 cp, about 50 cp to about 3,000 cp, about 50 cp to about 5,000 cp, about 50 cp to about 10,000 cp, about 50 cp to about 20,000 cp, about 50 cp to about 100,000 cp, about 100 cp to about 500 cp, about 100 cp to about 1,000 cp, about 100 cp to about 3,000 cp, about 100 cp to about 5,000 cp, about 100 cp to about 10,000 cp, about 100 cp to about 20,000 cp, about 100 cp to about 100,000 cp, about 500 cp to about 1,000 cp, about 500 cp to about 3,000 cp, about 500 cp to about 5,000 cp, about 500 cp to about 10,000 cp, about 500 cp to about 20,000 cp, about 500 cp to about 100,000 cp, about 1,000 cp to about 3,000 cp, about 1,000 cp to about 5,000 cp, about 1,000 cp to about 10,000 cp, about 1,000 cp to about 20,000 cp, about 1,000 cp to about 100,000 cp, about 3,000 cp to about 5,000 cp, about 3,000 cp to about 10,000 cp, about 3,000 cp to about 20,000 cp, about 3,000 cp to about 100,000 cp, about 5,000 cp to about 10,000 cp, about 5,000 cp to about 20,000 cp, about 5,000 cp to about 100,000 cp, about 10,000 cp to about 20,000 cp, about 10,000 cp to about 100,000 cp, or about 20,000 cp to about 100,000 cp. In some embodiments, the viscosity of the biocompatible patch material as it is transferred through the device is about 1 cp to about 10,000 cp. In some embodiments, the viscosity of the biocompatible patch material as it is transferred through the device is about 1 cp to about 5 cp, about 1 cp to about 10 cp, about 1 cp to about 20 cp, about 1 cp to about 50 cp, about 1 cp to about 100 cp, about 1 cp to about 500 cp, about 1 cp to about 1,000 cp, about 1 cp to about 5,000 cp, about 1 cp to about 10,000 cp, about 5 cp to about 10 cp, about 5 cp to about 20 cp, about 5 cp to about 50 cp, about 5 cp to about 100 cp, about 5 cp to about 500 cp, about 5 cp to about 1,000 cp, about 5 cp to about 5,000 cp, about 5 cp to about 10,000 cp, about 10 cp to about 20 cp, about 10 cp to about 50 cp, about 10 cp to about 100 cp, about 10 cp to about 500 cp, about 10 cp to about 1,000 cp, about 10 cp to about 5,000 cp, about 10 cp to about 10,000 cp, about 20 cp to about 50 cp, about 20 cp to about 100 cp, about 20 cp to about 500 cp, about 20 cp to about 1,000 cp, about 20 cp to about 5,000 cp, about 20 cp to about 10,000 cp, about 50 cp to about 100 cp, about 50 cp to about 500 cp, about 50 cp to about 1,000 cp, about 50 cp to about 5,000 cp, about 50 cp to about 10,000 cp, about 100 cp to about 500 cp, about 100 cp to about 1,000 cp, about 100 cp to about 5,000 cp, about 100 cp to about 10,000 cp, about 500 cp to about 1,000 cp, about 500 cp to about 5,000 cp, about 500 cp to about 10,000 cp, about 1,000 cp to about 5,000 cp, about 1,000 cp to about 10,000 cp, or about 5,000 cp to about 10,000 cp.

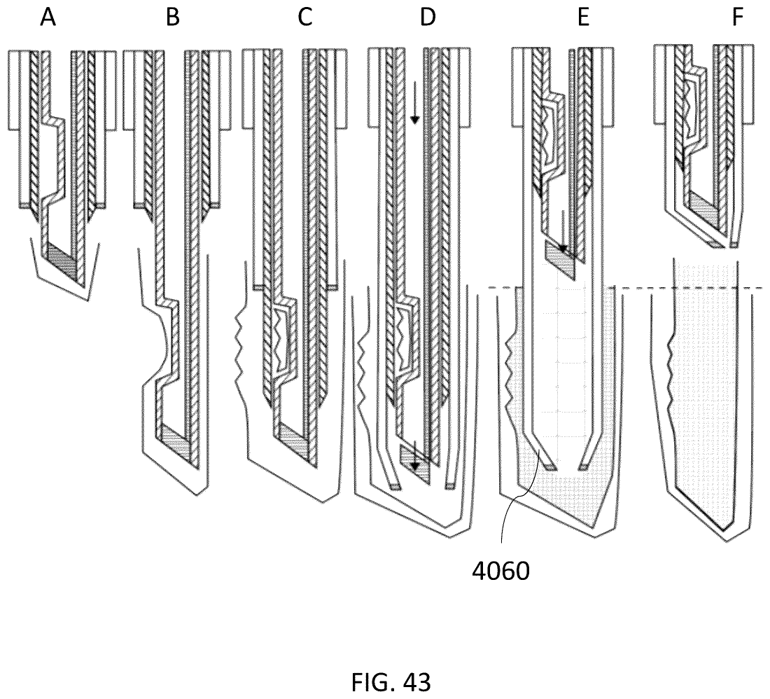

In some embodiments, the photopolymerized patch material is selectively, partial, mostly, or fully activated by photons while the patch material is still within a portion of the device, such that the material is “toughened” before its exposure to high pressured bleeding or tissues. An exemplary method of activation while within a portion of the device is depicted in FIGS. 43A-43E. This may facilitate rapidly sealing of actively bleeding tissue, sealing tissue bleeding at elevated or arterial blood pressures, sealing tissue in an impaired or dysfunctional coagulation system, adhesion to wet tissues or substances, prevention of dilution of the patch material, decreasing the Swelling Ratio (SR) of the patch material, increasing the viscoelastic strength of the patch material, increasing the viscosity of patch material, increase the contact area of monomers and polymers to the tissue, prevent local migration of patch material, preventing or limiting embolization of patch material, reducing the cure time for network growth or tissue seal, and reducing the time necessary for the needle or device to be in or exposed to organs or tissues, or some combination thereof.

In some embodiments, a mesh or structure is formed by photopolymerization. In some embodiments, microstructures are formed during an initial polymerization. In some embodiments, the patch material contains one, two, or more various types of molecules to create a homogenous, heterogenous, or mixed type of polymer structure. These molecules may include but are not limited to one or more proteins, lipids, sugars, amino acids, polysaccharides, glycosaminoglycans, sugars, alcohols, synthetic molecules, natural molecules, partially synthetic molecules, bio-adhesive type molecules, extracellular matrix type molecules, coagulant molecule, plant-based molecules, bacterial derived, animal derived, or composite molecules, other types of molecules, or some combinations thereof.

In some embodiments, the monomers comprise chemically active molecules attached via chemical bonding, mechanical bonding, or a combination thereof to increase the number of possible locations that can be activated. In some embodiments the added molecules are acryl groups, tyrosine groups, some other groups or some combination of these molecules or related molecules. In some embodiments, the patch material has elevated concentration of these molecules present to increase the cure rate of increase the mechanical strength or adhesion of the patch material.

In some embodiments, the patch material may be a “dual network” type photopolymerized mixture in some embodiments. This may include one or more different types of monomers, dimers, polymers, or some combination thereof used to create the 3D structure of the patch material upon curing. These molecules may contain similar or differing types of mechanical bond activity, chemical reactivity, bonding activity, curing speed, degradation time, hydrophobicity, electrical charge, shape, strength, elasticity, viscosity, viscoelasticity, burst pressure, breaking force, adhesiveness, shear strength, porosity, or uniformity.

### A. Rapidly Polymerizing, Highly Adhesive, Hypertensive Arteriostatic Biodegradable Patch Material

In some embodiments, the device comprises a patch material which is a dual-network (or multi-network) and is optimized for use in biopsy. In some embodiments, the patch material is a rapidly polymerizing, highly adhesive, hypertensive arteriostatic biodegradable patch material. The patch material may leverage the benefits of each of its main components in different ways. This embodiment of patch material will typically include one more low molecular weight biodegradable monomers which rapidly photopolymerizes under activation conditions, one or more higher molecular weight biodegradable monomer which contains one or more hydrophobic regions (or is modified to include the addition thereof), and one or more photoinitiators. This combination may be ideally suited for use with biopsy, especially for liver biopsies, kidney biopsies, pancreatic biopsies, and the like.

This unique combination leverages the rapid polymerization of the smaller monomer to rapidly strengthen the patch material and decrease the fluid turbulence related to arterial pressure bleeding to speed the connection between the internal 3-polymer structure and that at the surface of the tissue. The larger monomer acts as an anchor to the tissue, leveraging its large sizes to entangle with the molecules of the surface, and binds strongly to the surface through the hydrophobic interactions.

In some embodiments, the patch material mixture rapidly polymerizes despite arterial pressure bleeding to prevent secondary injury with the biopsy needle during the polymerization process. In some embodiments, the features of the patch material prevent major hemorrhage with every biopsy. In some embodiments, concentrations of the patch material elements are combined before or during the process of the procedure, such that when activated the rapidly polymerizing highly adhesive biodegradable patch material will nearly fully cure within 0.5 seconds, 1 seconds, 1.5 seconds, 2 seconds, 2.5 seconds, 3 seconds, 4 seconds, 5 seconds, 6 seconds, 7 seconds, 8 seconds, 9 seconds, 10 seconds, or within some other period considered rapid up to about 5 minutes, while also having high adhesive forces equal to or greater than that of hydrostatic force of hypertensive arterial pressures including 120 mmHG, 130 mmHG, 140 mmHG, 150 mmHG, 160 mmHG, 170 mmHG, up to about 300 mmHG, or more. In some embodiments, this patch material will typically contain at least one bond which can be cleaved in-situ, such that it is biodegradable within several months, but takes longer than 5 days to fully degrade in-situ.

In some embodiments, examples of the of the small molecular weight, rapidly polymerizing biodegradable monomer within the rapidly polymerizing, highly adhesive hypertensive arteriostatic patch material can include but are not limited to: gelatin, gelma, polyethylene glycol, PEGMA, PEGDA, oxidized alginate, oxidized alginate methylacrylate, tropoelastin, tropoelastin-methylacrylate, among various others. This component may alternatively be present as dimer, trimer, or small polymer form in the inactivated patch material. This component may have one or more methyl acrylate groups added to speed the reaction. In the appropriate concentrations, this monomer will fully polymerize when exposed to the appropriate light source in less than one or more of the following time periods: 0.5 seconds, 1 seconds, 1.5 seconds, 2 seconds, 2.5 seconds, 3 seconds, 4 seconds, 5 seconds, 6 seconds, 7 seconds, 8 seconds, 9 seconds, 10 seconds, or within some other period considered.

In some embodiments, the larger molecular weight monomer component of the rapidly polymerizing, highly adhesive, biodegradable photopolymerizable patch material is one or more of the following of molecules: glycosaminoglycans such as hyaluronic acid, collagen, chitosan, polyallylamine, proteins such as fibrinogen, laminin, some other monomer larger than the smaller monomer component, or some combination thereof. In some embodiments, this larger species has one or more hydrophobic segments, including but not limited to: a bio-inspired phenol group, some other phenol group, some other hydrocarbon chain, hydrocarbon ring, fatty-acid, glycerol, lipid, peptide segment with significantly externally exposed hydrophobic amino acid sequences, some other outwardly accessible hydrophobic segment, or some combination thereof. This hydrophobic can be an inherent portion of the larger molecule, a modification of another molecule, an addition before or during the patch forming process, or some combinations thereof.

The rapidly polymerizing, highly-adhesive, hypertensive arteriostatic biodegradable patch material may include one or more photoinitiator molecules such as those listed above. This patch material may include one or more other additional molecules described herein for additional benefits.

In some embodiments, this patch material is separated into component parts within a patch material cartridge, instrument cartridge, needle cartridge, syringe, dispenser, capsule, or some combination thereof. Alternatively, it may be drawn up or injected manually before, during, or after the procedure, such as by connecting a syringe to the introducer needle or some other port. The components of the rapidly polymerizing, highly adhesive, hypertensive arteriostatic biodegradable patch material may exist as a liquid, solid, semi-solid, dehydrated component, mixture, or some combination thereof. Additional cells, growth factors, antimicrobial, other components, or combinations thereof may also be a part of these mixtures in some embodiments.

In some embodiments, these rapidly polymerizing highly adhesive patch, hypertensive arteriostatic patch materials undergo some amount of initial exposure and activation within some portion of the device as described above. This preliminary activation can take place within the patch cartridge, needle cartridge, bore of the needle, lumen of the catheter, of some combination thereof. With this type of mixture, the smaller monomers can be activated allowing stabilization of the 3D matrix and partial polymerization of the small monomers to the large monomers, producing longer tissue anchors before extrusion into the wound site. This also may allow non-covalent bonding interactions, and resulting increase in facture propagation resistance to the patch material. It may also give more stability to the patch material matrix once extruded to allow closer interaction, more entanglement, more covalent, and more non-covalent bonds to form between the larger monomers and tissue.

In some embodiments, these rapidly polymerizing highly adhesive patch, hypertensive arteriostatic patch materials may comprise some other combination of components, solutions, additives, mixtures, accelerators, or otherwise. In some embodiments concentrations of single monomer or multi monomer patch mixtures may provide the following features: 1. Being injectable before, after, during, or in between biopsy procedures 2. Have the adhesive properties such that they are able to rapidly stop and prevent hemorrhage from hypertensive hydrostatic bleeding 3. Be biodegradable in not less than about 5 days to no more than about a year.

Specific examples which would benefit from these rapidly polymerizing, highly adhesive, hypertensive arteriostatic biodegradable patch material would include use in conjunction with all types of kidney and liver biopsies, especially in advancing disease is to have significant bleeding.

In some embodiments, this rapidly polymerizing, highly adhesive, hypertensive arteriostatic biodegradable patch material is utilized in combination with use of tissue alteration (such as RF monopolar, RF bipolar, or HF ultrasonic cautery). Use of rapidly polymerizing, highly adhesive, hypertensive arteriostatic patches (or similar mixtures) with tissue alteration may reduce the radial thickness of required cautery, increase adhesive forces, increase the speed of polymerization of this patch material, for more entanglement with denatured proteins and large monomer anchors, reduce the protective gel at the surface of tissue or biopsy site by desiccation of H₂O from native glycosaminoglycans (GAGs) and extracellular matrices (ECMs), reduce the amount of patch material required, reduce washout, improve tissue to polymer self-healing, improve wound healing speed, and significantly reduce risk of delayed bleeding.

In some embodiments, the device comprises a luminal body ligation clamp. In some embodiments, a combination of RF bipolar, impedance sensed/directed cautery is utilized in combination with the components, mechanisms, and controls for dispensing and activation of the of rapidly. This combination may be used for other types of needle-based procedures such as sealing arterial wounds after cardiac catheterization, treatment for chronic wounds, sealing surgical wounds such as ear-nose-throat (ENT) surgeries, and closure chronic fistulas.

A specific dual network patch material may include a moderately concentrated combination of a rapidly polymerizing molecule (such as GelMA) and a second monomer which includes a “bio-adhesive inspired” polyphenol chain. When expelled from the device near the site of instrumentation, the highly hydrophobic bio-adhesive polyphenol rapidly binds to the tissue walls before its full polymerization with other molecules. This rapid hydrophobic adhesion may act as a quick temporary sealant, slowing down the bleeding enough for platelet adhesion and the GelMA to form a 3d network and attach to the polyphenol sites.

In some embodiments, certain patch material cartridges (or other form) are selected by the user before the procedure with device. In some embodiments, the patch material is specific for platelet disfunction or other procedure with this risk factor present. In some embodiments, the patch material comprises concentrated oxidized alginate and hyaluronic acid are used. Oxidized alginate may be utilized and may be slower to degrade than most other types of patch material components, but still rapidly cures. In some embodiments, Hyaluronic acid can further strengthen the structure against elevated blood pressure bleeding above 120 mmHG, 160 mmHG, 180 mmHG, 200 mmHG or higher. providing a mix of rapid curing, toughness, and slow absorption.

In some embodiments, a patch material is configured to specifically towards resisting very high-pressured bleeding through rapid adhesion and high toughness. Patients with advanced kidney disease frequently have markedly high blood pressures (often over 220 mmHG) along with coagulation deficiencies. In some embodiments, the patch materials intended for these biopsies is composed of a concentrated mixture of GelMa and modified hyaluronic acid, with hyaluronic acid having been chemically altered to add a hydrophobic section to the molecule such as NB (N-(2-aminoethyl)-4-(4-(hydroxymethyl)-2-methoxy-5-nitrosophenoxy) butanamide). This creates a rapid seal (less than about 5 seconds) with high adhesion strength, burst pressure, or both.

In some embodiments, the patch material does not require photoactivation to polymerize or seal tissue. In some embodiments these patch materials are still able to be delivered and/or activated at the entire or portion of the site tissue, structures, or substances altered during instrumentation by the device. These patch materials may rely on a chemical, thermal, hydrophilic, ionic, pH, or some other mechanism of activation, plug, sealing, or coagulation method that does not require photoactivation to reach the desire effect, but still may be used to consistently prevent bleeding from arterial pressure. These patch materials may be comprised of one or more of the following substances: Vitagel, avista, TraumaStat, ChitoSam, HemCon, Axiostar, Celox, thrombin, transexamic acid, Floseal, Surgifoam, Gelfoam, Cyklopapron, quick clot, Erfa Tranexamic acid, other components, or a combination thereof.

In other embodiments, chemicals or substances are provided in the patch material to reduce the viscosity of the fluid so it can more easily flow through a small lumen. In some embodiments, accelerators can be mitigated using a combination of internal and external activation at the distal end of the device and near the target location. In some embodiments, components are used to selectively heat, cool, vibrate, alter, or otherwise modify the patch material to reduce the viscosity, toughness, and/or adhesion to facilitate deliver though a needle. In some embodiments, the patch material can be contained and delivered in a packet, capsule, pill, mesh, or other surrounding material or substance, which can be removed, destroyed, crushed, or expelled neat or at the target seal site.

Mixing of a patch material may include includes mixing with substances at various times or locations within or outside of the device to modulate curing time. In some embodiments, mixing includes mixing with substances at various times or locations within or outside of the device to modulate location of curing, viscosity, pressure on surrounding tissue, elasticity, strength (compressive, shear, and/or tensile), sensing ability, or combinations thereof.

In some embodiments, the patch material comprises tranexamic acid (TXA) liquids, solids, and or gels (a plant based product that promotes coagulation). In some embodiments, the patch material comprises PEGdma; PEGDA; PEGDA in combination with HA/GelMa/Collagen; PEGDA in combination with polyaniline PEGDA with biosensing capabilities; GelMa, GelMa with MeHA/heparin/gellan gum GelMa with nanosilicate/hydroxyapatite; GelMa with graphene oxide and carbon nanotubes; GelMa with Methacryloyl-substituted tropoelastin; GelMa with Dopamine GelMa with polyacrylamide and chitosan nanoparticles; MeHA; MeHA with methacrylate chondroitin sulfate, devitalized cartilage microparticles, and/or N-cadherin mimetic peptides; MeHA with puramatrix peptide. In some embodiments, the polymerization reaction is triggered by radically reacting photoinitiators during laser irradiation, which allows real time monitoring of polymerization. In some embodiments, the patch material comprises SR499. In some embodiments, the polymerization reaction is triggered by radically reacting photoinitiators during laser irradiation, which allows real time monitoring of polymerization.

In some embodiments, the patch material comprises Microspheres, gelatin-based products used alone or in combination with a pro-coagulant substance cellulose-based haemostatic agents, fibrin, synthetic glues, adhesives, pledgets, nanoparticles, microparticles, zeolite, smectite, sponges, and combinations thereof. In some embodiments, polysaccharide-based haemostatics are utilized. Haemostatics may include N-acetyl-glucosamine-containing glycosaminoglycans purified from microalgae and diatoms and microporous polysaccharide haemospheres produced from potato starch. A patch material may also include systemically delivered medications that help and/or promote the hemostasis of the procedure, which is delivered by IV, IM, IO local to the location or remotely. Photoactivated patch material can be composed of photoactivated or inert polysaccharides, fibrin molecules, fibrinogen molecules.

In some embodiments, a mesh, plug, solid, or highly viscous or viscoelastic substance is used in combination with the patch material. In some embodiments, the patch material is extruded from coaxial needles such that multiple layers contain different substances with potentially different properties. In some embodiments, the patch material comprises ECM. In some embodiments, the patch material comprises various architectural forms and compositions in different tissues. In some embodiments, the patch material comprises a complex 3D network consisting of mainly collagen and elastic fibers, which also contain proteoglycans, multi-adhesive proteins (e.g., fibronectin, laminin), and glycosaminoglycans (e.g., hyaluronan).

In some embodiments, the patch material comprises is photoinitiated by laser-based stereolithography and digital light projection (DLP). In some embodiments, photoinitiation of the patch material comprises visible-light curing of poly(2-hydroxyethyl methacrylate) (p-HEMA) hydrogel under 405 nm light+/−sodium alginate.

In some embodiments, the patch material comprises nanoparticles. Nanoparticles may be utilized for storing and releasing other molecules. The rate, amount, and timing can be modified in various ways, and may be caused by specific stimulation. For example, the nanostructures, in some embodiments, activation can be stimulated by a drop in pH to release antimicrobials (antibiotics). Bacterial presence can cause a local decrease in pH, which would stimulate the nanoparticles in the example to release substances to eliminate the bacteria. Nanoparticles may also release cells or cellular stimulating molecules. Stimulating molecules may be utilized to release molecules stimulating the generation of new blood vessels and glomeruli when placed during kidney biopsy. They may also be placed with stem cells or other growth factors upon sealing the kidney tissue after biopsy. The released substances could also help differentiate those stem cells to become and divide into certain cell lines and structures. Nano particles utilized may include silicate nanoparticles, ceramic nanoparticles, carbon nanotubes, thermal sensitive nanoparticles, photon sensitive nanoparticles, nanoparticles with other sensitivity to stimuli (e.g., pressure, cooling, heating, etc.), and combinations thereof.

In some embodiments, a combination of photoactivated bonded substances is mixed with, bonded to, isolated, or related to chemicals that promote blood clot formation, such that the combination of the sealing chemicals and chemicals that promote clotting. In some embodiments, the combination of substances prevents and/or decreases the amount of clotting that occurs in other parts of the organ or organs away from the instrumented portion, such as preventing clotting or material buildup in kidney tubules, the bile duct system, lymphatic system, blood vessels of otherwise.

In some embodiments, the patch material is modified in some manner to change the spectrum of wavelengths by which chemical bonds are created or broken. For example, a substance that normally only creates bonds when exposed to photons of the UV spectrum, may be chemically altered to create bonds within the visible spectrum. These changes can be produced by one or more chemical, electrical, or mechanical reactions or in some other way.

### B. Repair Structure

In some embodiments, the patch material comprises a gel, solid cast, liquid, partitioned segments, tape, mesh, mixed gel, capsule, packet, plug, powder, cream, or a combination thereof. In some embodiments, the patch material comprises one or more monomer, dimer, and/or polymer components and crosslinkers. In some embodiments, polymer components and cross linkers are combined and/or bonded before, during, and/or after the procedure. In some embodiments, the patch material comprises gelatin, alginate, fibrin, silk fibers, ethylene glycol, polyethylene glycol, collagen (type one, two, four, and/or modified), chitosan, hyaluronic acid, genipin and genipin scaffolding, nucleic acids such as DNA, RNA, and modified versions, plant originating molecules, cellulose, indocyanine-green polymers, polysaccharides such as cellulose, dextran, agarose, biosentry hydrogel, Taracel T hydrogel, SpaceOAR Hydrogel, Fibrinogen, Albumin, Evicel, Coseal, n-vinylamide and hydroxyl, hydroxyalkyl methacrylate, methylacrylamide, hydrogels with one, two, or more methods of stimulating a response to stimuli, Ventrigel, silica based hydrogels, Polymerizing components within other substances, polyethylene oxide, bioadhesive hydrogels, oxidized pullulan, carboxyl containing hydrogel, Cellulose, Cellulose-silica based hydrogel, polymeric fibers which may or may not be bound to hydrogel, Poly alkylene glycol, antimicrobial hydrogel, lyophilized hyaluronic acid, Hypromellose, nano-fiber hydrogel hybrid, hydroxyethyl cellulose, Chitosan-grafted dihydrocaffinic acid, Alginate chitosan, drug loaded micelles, collagen with chitosan, Polyacrylamide and hyaluronic acid, Polyacrylonitrile, hydroxymethylcellulose, radiopaque substances, ABA triblockcopolymers of vit D, Functionalized Polycarbonate, polymer-nanoparticle, chondoitan sulfate, hylauronin, Polester based polymers: PLA, PCL, PGS, PU, Conducting Polymers: PPY, PTh, pani, bioactuators, Biosensors: PLC-collagen nanofibers, metal and metal oxide nanoparticles, Titanium Oxide nanoparticles, graphene, graphene Oxide-Alginate, Elastin, Tropoelastin, Alg-MA (alginate methylacrylate), MeTro (Methylacrymide pendant of tropoelastin), Alginate, B-D-mannuronic acid, a-L-guluronic acid, Albumin, Evicel, Coseal, Tisseel, Tyrosine rich molecules, Molecules modified to contain an increased amount of tyrosine molecules, Mussel Adhesive Proteins (MAPs) (natural, synthetic, recombinant, and modified) such as LAMBA, 3,4-dihydroxyphenylalanine (DOPA) rich molecules, Polyacrylamide, poly-vinyl alcohols, Hybrid molecules, Chitin, Chitosan, Catechols, 3,4-dihydroxybenzaldehyde, Benzyl azide, carbodiimide, heparin, gelatin gum, carbon nanotubes, other Components of extracellular matrices of with various architectural forms and compositions, in different tissues, a complex 3D network consisting of mainly collagen and which also may contain elastic fibers, multi-adhesive proteins (e.g., fibronectin, laminin), proteoglycans, glycosaminoglycans (e.g., hyaluronan), N-3-dimethylaminopropyl-N-ethylcarbodiimide hydrochloride (EDC), phenolated molecules, chondroitin sulfate (CS), CS-MA, oxCS-MA (oxidized with periodate), poly(2-hydroxyethylmethacrylate) (PolyHEMA), Dextran (Polysacharide), DexMA, OxDex-MA-Gel, DMSO, 2-isocyanatoethylmethacrylate, 2-nitrobenzyl alcohol, Polyethyleneimine, E-Poly(L-lysine), bovine serum albumin, PRP (Platelet rich plasma), oNB-CMC, Carboxymethyl cellulose, N-aliphatic diethanolamide (soy diol), co-polyesters, 2,2-dimethoxy-2-phenylacetatephene, Alginate with calcium lactate, tyrosine kinase inhibitors, Copolymers with vit E, glutathione crosslinker, Thiols, In some embodiments, the patch material comprises an elastic composite hydrogel based on methacryloyl elastin-like polypeptide (mELP) combined with gelatin methacryloyl (GelMA) and a stiff glue based on pure GelMA. Using multiple gels may allow advantages from different properties: such as an elastic composite hydrogel based on methacryloyl elastin-like polypeptide (mELP) combined with gelatin methacryloyl (GelMA) and a stiff glue based on pure GelMA. Two formulations with distinct mechanical characteristics may be necessary to achieve stable anastomosis.

In some embodiments, the patch material comprises GelMA, GelMA-MeHA/heparin/gelitan gum, GelMA-nanosilcate/hydroxyapetite, GelMA-Graphine oxide/Carbon nanotubes, Gelma-methylacryloyl substituted tropoelastin, GelMA-Dopamine, GelMA-Polyacrylamide/Chitosen Nanoparticles, GelMA-nickel nanoparticles+reduced graphene oxide, Gel-St (gelatin functionalized with styrene groups), GelDA, PEGDA (Polyethyleneglycol diacrylate), PEGDA-HA/GelMA/Collagen, MeHA, MeHA Puramatrix Peptide, MeHA-methylacrylated chondroitin sulfate/n-cadherin memetic peptides, PegMa, PegMA-Polyaniline, Gel-X (xanthene conjugated gelatin), benzophenone conjugated gelatin (Gel-BPh), oxidized variations of molecules to improve their adhesive properties (especially under wet conditions), sodium periodate (used at times to oxidize), IDCG-Chitosan, Gelatin conjugated with pendant catechol groups (Gel-Cat), Phloretic acid, Gelatin with pendant phenol (Gel-Phe), Methacrylic Anhydride, 2-hydroxy-4′-(2-hydroxyethoxy)-2-methylpropiophenone (Igacure 2959), PEG-dimethylacrylate, Thiol pendant chiotosen, Glycol chitosan (G-CH), AZ-CH-LA, CH-MA-Cat, Ethylene Glycol, 3-armed PEG with added Catechol, or combinations thereof.

In some embodiments the patch material comprises natural polymer hydrogels and/or synthetic polymer hydrogels. Synthetic hydrogels may include poly(lactic acid) (PLA), poly(glycolic acid) (PGA), poly(lactic-co-glycolic acid) (PLGA), poly(ethylene glycol) (PEG), poly(c-caprolactone) (PCL), poly(2-hydroxyethyl methacrylate) (p-HEMA). In some embodiments, the patch material comprises photo-cured biopsy hemostasis, photo-sealed biopsy hemostasis, epoxy-based materials, SU 8, CAR 44, acrylate based materials, ormocer, or a combination thereof. In some embodiments, the patch material comprises inorganic/organic hybrid polymers. In some embodiments, patch materials are synthesized by sol-gel processing, whereby inorganic units are connected to organic moieties on a molecular level. In some embodiments, the polymers are hybrids. In some embodiments, hybrid materials combine the properties of organic polymers (low-temperature processing, functionality, and toughness) with those of glasslike materials (hardness, chemical and thermal stability, and transparency). This may allow one to achieve material properties not accessible with composite or polymer materials.

### II. Form Factor

### A. Needle

In some embodiments, the needle comprises a Chiba needle, a Turner needle, a Madayag needle, a Spinal Needle, a Greene needle, a Franseen needle, a Wescott needle, a side-cutting Tru-Cut biopsy needle, a rotating needle, a Center-cutting biopsy needle, a Full-core biopsy needle, a Lancet Tip biopsy needle, biopsy needle or system utilizing vacuum components, fine needle aspiration needle, or a combination of features thereof.

### 1. Sheath

In some embodiments the device comprises a sheath. In some embodiments, the sheath encloses a sample tray. In some embodiments, the sheath is configured as a cutting sheath. In some embodiments, the sheath seals one or more apertures for emitting the patch material. In some embodiments, the sheath covers a reservoir containing the patch material. In some embodiments, there is a stabilizing sheath, that is fixedly connected to one or more portions of the device on its proximal end. On its distal end, it terminates a short distance from the distal tip of the needle, approximately 5 cm from the needle tip though this may vary from about 2 cm to about 10 cm from the tip. In some embodiments, the sheath stabilizes the apparatus and provides an anchor point, such that the inner or outer biopsy needle, catheter, light carrying components, or patch of some other portion of the device can be smoothly retracted towards the body of the device from a more distal location to a more proximal location, while not pulling the handle towards the interior of the patient. This helps ensure correct curing time as well, and is an important aspect for curing the patch material to a high adhesion and toughness necessary to stop and seal hypertensive arterial pressure bleeding.

### 2. Movement of Needle and Sheath

In some embodiments, the device comprises an actuator for controlling advancement and or retraction of the needle. In some embodiments, the actuator is a piezoelectric actuator. In some embodiments, movement of the sheath is automated. In some embodiments, movement of the needle is automated. In some embodiments, automated movement of the sheath controls the speed and distance of the movement. In some embodiments, automated movement of the needle controls the speed and distance of the movement.

In some embodiments, as depicted in FIGS. 5A-5D, a sheath comprises one or more portions which rotate to cut and enclose a tissue sample with a sample tray 530. The rotating portions may be referred to as wings. In some embodiment, the ends of the wings are provided with a sharpened cutting surface 505.

### 3. Introducer Needle

In some embodiments, an introducer needle is utilized. The introducer needle may first be in introduced proximal of a target site for a biopsy. A biopsy needle may then be introduced through the introducer needle. The biopsy needle may then be utilized to perform the biopsy and enclose a tissue sample within the trough of the biopsy needle. After the biopsy is performed, a repair needle may be guided through the introducer needle to be provided at the biopsy site. Once at the biopsy site, the repair needle may inject biocompatible patch material to the biopsy site. In some embodiments, the repair needle comprises a light emitting surface formed by a wave guides guiding a light source to the distal tip of the needle. The light source may be activated to initiate photopolymerization of the biocompatible patch material at the biopsy site.

### B. Light Source

In some embodiments, photons are used to control some portion of the device. In some embodiments, photons emission is timed with needle insertion or retraction, timing of photoactivated substance release, amount of photoactivated substance release, control of shape of 3D printed object, or a combination thereof.

In some embodiments, the device is comprised of photocuring components which include: one two or more light generating sources, light transmitting components, waveguides, connectors, couplers, filters, projectors, diffusers, optical fibers, optical sensors, thermal sensors, positional sensors, emitters, fiber optics, fiber cores, grates, filters, shutters, fiber cladding, waveguide cladding, the components in which the optical components are attached or embedded, the catheter, wires, cables, connectors, waveguides, photon sensing components, computing devices, controlling devices, attachments to control movement of some of these components, components to produce movement of these components, components to couple movement of these components to other movements, automated controls, manual controls, semi-automated controls, needles, patch materials, or other light transmission related components, or some combination thereof.

In some embodiments, the device is comprised of one or more photon generating sources located in one or more of the following locations: within the housing of the device, external to the housing of the device, within a remote unit, external to a remote unit, in the handle of the device, outside of the handle, a removeable internal module, a permanent internal module, a removeable external module, a permanent external module, within the lumen of the internal needle, within the lumen of the external needle, withing the lumen of the advanced catheter, within the lumen of the stabilizing catheter, within the body of the needle, within the body of the catheter, some other location, or a combination there up, In some embodiments, the light source is permanently attached, temporarily attached, reusable, disposable, autoclavable, rechargeable, partially disposable, partially reusable, cleaned with antiseptic, covered in a drape, covered in a sterile sleeve, covered in a sterile capsule, otherwise modified, or a combination thereof.

In some embodiments, the light source may comprise an electrochemical light source, a gas light source (e.g. gas laser), a chemical source, a laser source, a LED (light-emitting diode), a solar source, a mechanic electrical source, a tungsten lamp, a tungsten-halogen lamp; a high-pressure gas discharge lamp; an arc lamps (e.g. xenon, or mercury-xenon, high-pressure lamps); a low-pressure discharge lamps, a special phosphor fluorescent lamp (e.g., UV fluorescent lamps), other continuous sources (e.g. LEDs and synchrotrons), pulsed sources (e.g. flashlamps), two-photon polymerization with ultrashort laser pulses (e.g. as used for nanoscale 3d printing), or combinations thereof. In some embodiments, the device comprises a protective light filtering component such as a filter, grating, shudder, shade, other component, or a combination there of to limit the exposure of biological tissues to dangerous frequencies.

The laser or beam light may be emitted by a laser. The laser light may be emitted by a continuous wave laser. The laser light may be emitted by a pulsed laser. The laser light may be emitted by a gas laser, such as a helium-neon (HeNe) laser, an argon (Ar) laser, a krypton (Kr) laser, a xenon (Xe) ion laser, a nitrogen (N2) laser, a carbon dioxide (CO2) laser, a carbon monoxide (CO) laser, a transversely excited atmospheric (TEA) laser, or an excimer laser. For instance, the laser light may be emitted by an argon dimer (Ar2) excimer laser, a krypton dimer (Kr2) excimer laser, a fluorine dimer (F2) excimer laser, a xenon dimer (Xe2) excimer laser, an argon fluoride (ArF) excimer laser, a krypton chloride (KrCl) excimer laser, a krypton fluoride (KrF) excimer laser, a xenon bromide (XeBr) excimer laser, a xenon chloride (XeCl) excimer laser, or a xenon fluoride (XeF) excimer laser. The laser light may be emitted by a dye laser.

The laser light may be emitted by a metal-vapor laser, such as a helium-cadmium (HeCd) metal-vapor laser, a helium-mercury (HeHg) metal-vapor laser, a helium-selenium (HeSe) metal-vapor laser, a helium-silver (HeAg) metal-vapor laser, a strontium (Sr) metal-vapor laser, a neon-copper (NeCu) metal-vapor laser, a copper (Cu) metal-vapor laser, a gold (Au) metal-vapor laser, a manganese (Mn) metal-vapor, or a manganese chloride (MnCl2) metal-vapor laser.

The laser light may be emitted by a solid-state laser, such as a ruby laser, a metal-doped crystal laser, or a metal-doped fiber laser. For instance, the laser light may be emitted by a neodymium-doped yttrium aluminum garnet (Nd:YAG) laser, a neodymium/chromium doped yttrium aluminum garnet (Nd/Cr:YAG) laser, an erbium-doped yttrium aluminum garnet (Er:YAG) laser, a neodymium-doped yttrium lithium fluoride (Nd:YLF) laser, a neodymium-doped yttrium orthovanadate (ND:YVO4) laser, a neodymium-doped yttrium calcium oxoborate (Nd:YCOB) laser, a neodymium glass (Nd:glass) laser, a titanium sapphire (Ti:sapphire) laser, a thulium-doped ytrrium aluminum garnet (Tm:YAG) laser, a ytterbium-doped ytrrium aluminum garnet (Yb:YAG) laser, a ytterbium-doped glass (Yt:glass) laser, a holmium ytrrium aluminum garnet (Ho:YAG) laser, a chromium-doped zinc selenide (Cr:ZnSe) laser, a cerium-doped lithium strontium aluminum fluoride (Ce:LiSAF) laser, a cerium-doped lithium calcium aluminum fluoride (Ce:LiCAF) laser, a erbium-doped glass (Er:glass), an erbium-ytterbium-codoped glass (Er/Yt:glass) laser, a uranium-doped calcium fluoride (U:CaF2) laser, or a samarium-doped calcium fluoride (Sm:CaF2) laser.

The laser light may be emitted by a semiconductor laser or diode laser, such as a gallium nitride (GaN) laser, an indium gallium nitride (InGaN) laser, an aluminum gallium indium phosphide (AlGaInP) laser, an aluminum gallium arsenide (AlGaAs) laser, an indium gallium arsenic phosphide (InGaAsP) laser, a vertical cavity surface emitting laser (VCSEL), or a quantum cascade laser.

The laser light may be continuous wave laser light. The laser light may be pulsed laser light. The laser light may have a pulse length of at least 1 femtoseconds (fs), at least 2 fs, at least 3 fs, at least 4 fs, at least 5 fs, at least 6 fs, at least 7 fs, at least 8 fs, at least 9 fs, at least 10 fs, at least 20 fs, at least 30 fs, at least 40 fs, at least 50 fs, at least 60 fs, at least 70 fs, at least 80 fs, at least 90 fs, at least 100 fs, at least 200 fs, at least 300 fs, at least 400 fs, at least 500 fs, at least 600 fs, at least 700 fs, at least 800 fs, at least 900 fs, at least 1 picosecond (ps), at least 2 ps, at least 3 ps, at least 4 ps, at least 5 ps, at least 6 ps, at least 7 ps, at least 8 ps, at least 9 ps, at least 10 ps, at least 20 ps, at least 30 ps, at least 40 ps, at least 50 ps, at least 60 ps, at least 70 ps, at least 80 ps, at least 90 ps, at least 100 ps, at least 200 ps, at least 300 ps, at least 400 ps, at least 500 ps, at least 600 ps, at least 700 ps, at least 800 ps, at least 900 ps, at least 1 nanosecond (ns), at least 2 ns, at least 3 ns, at least 4 ns, at least 5 ns, at least 6 ns, at least 7 ns, at least 8 ns, at least 9 ns, at least 10 ns, at least 20 ns, at least 30 ns, at least 40 ns, at least 50 ns, at least 60 ns, at least 70 ns, at least 80 ns, at least 90 ns, at least 100 ns, at least 200 ns, at least 300 ns, at least 400 ns, at least 500 ns, at least 600 ns, at least 700 ns, at least 800 ns, at least 900 ns, at least 1,000 ns, or more. The laser light may have a pulse length that is within a range defined by any two of the preceding values. For instance, the laser light may have a pulse length between 1 ns and 50 ns.

The laser light may have a repetition rate of at least 1 hertz (Hz), at least 2 Hz, at least 3 Hz, at least 4 Hz, at least 5 Hz, at least 6 Hz, at least 7 Hz, at least 8 Hz, at least 9 Hz, at least 10 Hz, at least 20 Hz, at least 30 Hz, at least 40 Hz, at least 50 Hz, at least 60 Hz, at least 70 Hz, at least 80 Hz, at least 90 Hz, at least 100 Hz, at least 200 Hz, at least 300 Hz, at least 400 Hz, at least 500 Hz, at least 600 Hz, at least 700 Hz, at least 800 Hz, at least 900 Hz, at least 1 kilohertz (kHz), at least 2 kHz, at least 3 kHz, at least 4 kHz, at least 5 kHz, at least 6 kHz, at least 7 kHz, at least 8 kHz, at least 9 kHz, at least 10 kHz, at least 20 kHz, at least 30 kHz, at least 40 kHz, at least 50 kHz, at least 60 kHz, at least 70 kHz, at least 80 kHz, at least 90 kHz, at least 100 kHz, at least 200 kHz, at least 300 kHz, at least 400 kHz, at least 500 kHz, at least 600 kHz, at least 700 kHz, at least 800 kHz, at least 900 kHz, at least 1 megahertz (MHz), at least 2 MHz, at least 3 MHz, at least 4 MHz, at least 5 MHz, at least 6 MHz, at least 7 MHz, at least 8 MHz, at least 9 MHz, at least 10 MHz, at least 20 MHz, at least 30 MHz, at least 40 MHz, at least 50 MHz, at least 60 MHz, at least 70 MHz, at least 80 MHz, at least 90 MHz, at least 100 MHz, at least 200 MHz, at least 300 MHz, at least 400 MHz, at least 500 MHz, at least 600 MHz, at least 700 MHz, at least 800 MHz, at least 900 MHz, at least 1,000 MHz, or more. The laser light may have a repetition rate that is within a range defined by any two of the preceding values.

The laser light may have a pulse energy of at least 1 nanojoule (nJ), at least 2 nJ, at least 3 nJ, at least 4 nJ, at least 5 nJ, at least 6 nJ, at least 7 nJ, at least 8 nJ, at least 9 nJ, at least 10 nJ, at least 20 nJ, at least 30 nJ, at least 40 nJ, at least 50 nJ, at least 60 nJ, at least 70 nJ, at least 80 nJ, at least 90 nJ, at least 100 nJ, at least 200 nJ, at least 300 nJ, at least 400 nJ, at least 500 nJ, at least 600 nJ, at least 700 nJ, at least 800 nJ, at least 900 nJ, at least 1 microjoule (μJ), at least 2 μJ, at least 3 μJ, at least 4 μJ, at least 5 μJ, at least 6 μJ, at least 7 μJ, at least 8 μJ, at least 9 μJ, at least 10 μJ, at least 20 μJ, at least 30 μJ, at least 40 μJ, at least 50 μJ, at least 60 μJ, at least 70 μJ, at least 80 μJ, at least 90 μJ, at least 100 μJ, at least 200 μJ, at least 300 μJ, at least 400 μJ, at least 500 μJ, at least 600 μJ, at least 700 μJ, at least 800 μJ, at least 900 μJ, a least 1 millijoule (mJ), at least 2 mJ, at least 3 mJ, at least 4 mJ, at least 5 mJ, at least 6 mJ, at least 7 mJ, at least 8 mJ, at least 9 mJ, at least 10 mJ, at least 20 mJ, at least 30 mJ, at least 40 mJ, at least 50 mJ, at least 60 mJ, at least 70 mJ, at least 80 mJ, at least 90 mJ, at least 100 mJ, at least 200 mJ, at least 300 mJ, at least 400 mJ, at least 500 mJ, at least 600 mJ, at least 700 mJ, at least 800 mJ, at least 900 mJ, a least 1 Joule (J), or more. The laser light may have a pulse energy that is within a range defined by any two of the preceding values. For instance, the laser light may have a pulse energy between 100 mJ and 500 mJ.

The laser light may have an average power of at least 1 microwatt (μV), at least 2 μW, at least 3 μW, at least 4 μW, at least 5 μW, at least 6 μW, at least 7 μW, at least 8 μW, at least 9 μW, at least 10 μW, at least 20 μW, at least 30 μW, at least 40 μW, at least 50 μW, at least 60 μW, at least 70 μW, at least 80 μW, at least 90 μW, at least 100 μW, at least 200 μW, at least 300 μW, at least 400 μW, at least 500 μW, at least 600 μW, at least 700 μW, at least 800 μW, at least 900 μW, at least 1 milliwatt (mW), at least 2 mW, at least 3 mW, at least 4 mW, at least 5 mW, at least 6 mW, at least 7 mW, at least 8 mW, at least 9 mW, at least 10 mW, at least 20 mW, at least 30 mW, at least 40 mW, at least 50 mW, at least 60 mW, at least 70 mW, at least 80 mW, at least 90 mW, at least 100 mW, at least 200 mW, at least 300 mW, at least 400 mW, at least 500 mW, at least 600 mW, at least 700 mW, at least 800 mW, at least 900 mW, at least 1 watt (W), at least 2 W, at least 3 W, at least 4 W, at least 5 W, at least 6 W, at least 7 W, at least 8 W, at least 9 W, at least 10 W, at least 20 W, at least 30 W, at least 40 W, at least 50 W, at least 60 W, at least 70 W, at least 80 W, at least 90 W, at least 100 W, at least 200 W, at least 300 W, at least 400 W, at least 500 W, at least 600 W, at least 700 W, at least 800 W, at least 900 W, at least 1,000 W, or more. The laser light may have a power that is within a range defined by any two of the preceding values.

The laser light may comprise a wavelength in the ultraviolet (UV), visible, or infrared (IR) portions of the electromagnetic spectrum. The laser light may comprise a wavelength of at least 100 nanometers (nm), at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm, at least 300 nm, at least 310 nm, at least 320 nm, at least 330 nm, at least 340 nm, at least 350 nm, at least 360 nm, at least 370 nm, at least 380 nm, at least 390 nm, at least 400 nm, at least 410 nm, at least 420 nm, at least 430 nm, at least 440 nm, at least 450 nm, at least 460 nm, at least 470 nm, at least 480 nm, at least 490 nm, at least 500 nm, at least 510 nm, at least 520 nm, at least 530 nm, at least 540 nm, at least 550 nm, at least 560 nm, at least 570 nm, at least 580 nm, at least 590 nm, at least 600 nm, at least 610 nm, at least 620 nm, at least 630 nm, at least 640 nm, at least 650 nm, at least 660 nm, at least 670 nm, at least 680 nm, at least 690 nm, at least 700 nm, at least 710 nm, at least 720 nm, at least 730 nm, at least 740 nm, at least 750 nm, at least 760 nm, at least 770 nm, at least 780 nm, at least 790 nm, at least 800 nm, at least 810 nm, at least 820 nm, at least 830 nm, at least 840 nm, at least 850 nm, at least 860 nm, at least 870 nm, at least 880 nm, at least 890 nm, at least 900 nm, at least 910 nm, at least 920 nm, at least 930 nm, at least 940 nm, at least 950 nm, at least 960 nm, at least 970 nm, at least 980 nm, at least 990 nm, at least 1,000 nm, at least 1,010 nm, at least 1,020 nm, at least 1,030 nm, at least 1,040 nm, at least 1,050 nm, at least 1,060 nm, at least 1,070 nm, at least 1,080 nm, at least 1,090 nm, at least 1,100 nm, at least 1,110 nm, at least 1,120 nm, at least 1,130 nm, at least 1,140 nm, at least 1,150 nm, at least 1,160 nm, at least 1,170 nm, at least 1,180 nm, at least 1,190 nm, at least 1,200 nm, at least 1,210 nm, at least 1,220 nm, at least 1,230 nm, at least 1,240 nm, at least 1,250 nm, at least 1,260 nm, at least 1,270 nm, at least 1,280 nm, at least 1,290 nm, at least 1,300 nm, at least 1,310 nm, at least 1,320 nm, at least 1,330 nm, at least 1,340 nm, at least 1,350 nm, at least 1,360 nm, at least 1,370 nm, at least 1,380 nm, at least 1,390 nm, at least 1,400 nm, or more. The laser light may comprise a wavelength that is within a range defined by any two of the preceding values.

The laser light may have a bandwidth of at least 0.001 nm, at least 0.002 nm, at least 0.003 nm, at least 0.004 nm, at least 0.005 nm, at least 0.006 nm, at least 0.007 nm, at least 0.008 nm, at least 0.009 nm, at least 0.01 nm, at least 0.02 nm, at least 0.03 nm, at least 0.04 nm, at least 0.05 nm, at least 0.06 nm, at least 0.07 nm, at least 0.08 nm, at least 0.09 nm, at least 0.1 nm, at least 0.2 nm, at least 0.3 nm, at least 0.4 nm, at least 0.5 nm, at least 0.6 nm, at least 0.7 nm, at least 0.8 nm, at least 0.9 nm, at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, or more. The laser light may have a bandwidth that is within a range defined by any two of the preceding values.

The laser light may have a diameter (for instance, as measured by a Rayleigh beam width, full width at half maximum, 1/e² width, second moment width, knife-edge width, D86 width, or any other measure of beam diameter) of at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 6 mm, at least 7 mm, at least 8 mm, at least 9 mm, at least 10 mm, at least 20 mm, at least 30 mm, at least 40 mm, at least 50 mm, at least 60 mm, at least 70 mm, at least 80 mm, at least 90 mm, at least 100 mm, or more. The first light may have a diameter of at most 100 mm, at most 90 mm, at most 80 mm, at most 70 mm, at most 60 mm, at most 50 mm, at most 40 mm, at most 30 mm, at most 20 mm, at most 10 mm, at most 9 mm, at most 8 mm, at most 7 mm, at most 6 mm, at most 5 mm, at most 4 mm, at most 3 mm, at most 2 mm, at most 1 mm, at most 0.9 mm, at most 0.8 mm, at most 0.7 mm, at most 0.6 mm, at most 0.5 mm, at most 0.4 mm, at most 0.3 mm, at most 0.2 mm, at most 0.1 mm, or less.

In some embodiments, the device uses a specific laser photocoagulation system for depending on the part of the body, pathophysiological condition. In some embodiments, the device is configured to perform photocoagulation with wavelengths specific to coagulating blood, ablating vessels, desiccating water, cautery, or some combination thereof. The waveguides for these embodiments use one or more waveguides which permit transmission of this photocoagulation light.

In some embodiments, the device uses a photocoagulation to alter the surface of a wound followed by the application of patch material. In some embodiments, this patch material is highly adhesive, rapidly curing photo-polymerizing hydrogel. In some embodiments, the device comprises more than one light source such that is able to both perform both photocoagulation and curing photopolymerized gel.

In some embodiments, the device is comprised of a light source connected to a rapidly extendable power source. In some embodiments, this rapidly extending power source can be a telescoping circuitry system in the handle, power cartridge, instrument cartridge, needle cartridge, or some other portion of the device. In some embodiments, the device can have a rapidly extendable power source through an electronic connection to an instrument cartridge through a component also connecting driving forces of one or more actuators to the instrument cartridge or needle cartridge. In some embodiments, the rapidly extendable portions of the device are comprised of low mass parts that do not add considerable weight to the components, translating motion through a driver system to perform a biopsy. In some embodiments, the rapidly extendable power system includes portions with low friction coatings to allow sliding translation with limited increase in required force. In some embodiments, using a reusable power source, the rapidly extended portion of the device is incorporated in the reusable section when using hypertensive ateriostatic gels, as such gels have high adhesion forces and can quicky disrupt any telescoping or extendable portions of the biopsy needle translation.

### 1. Optimal Thermal or Vibrational Resistance of Light Transmitting Catheter for Use in Combo Device

In some embodiments, one or more of the light transmitting components of the catheter are comprised one or more of types of glass, plastic, silica materials, non-silica material, polymer optical fibers, Fontex, synthetic material, natural material, hydrogel, elastomer, some other class of material, or a combination of these classes of materials. In some embodiments, one of more of the light transmitting of the components is comprised of one or more material optimized for use in one or more specific types of procedures, biopsies, patch materials, surgeries, endoscopies, bronchoscopy, wound treatments, organs, tissues, substances, disease states, size, shape, width, mechanical properties, flexibility, bend stiffness, elasticity, tensile strength, shear strength, breaking strength, melting point, softness, electrical conductance or resistance (or impedance), thermal conductance or resistance, vibrational conductance or resistance, light transmission properties, light propagation loss, light attenuation, light frequency, light biocompatibility, light power, nontoxicity, degradation time, self-healing properties, wavelengths, power, or some combination thereof.

In some embodiments, there is an unanticipated advantage to comprising one or more of the light transducing materials with stretchable optical fibers. Some embodiments need to be light weight for precise control, prolonged use (especially when there are patch cure times greater than 5-10 seconds), or other reasons. In some embodiments, one of more components comprising a light transducing component is a highly elastic, stretchable polymer based optical fibers, allows for axial movement of the light transmitting components without the need for redundant lengths of optical fibers. This advantage is especially important to embodiments designed for using photopolymerized patch material in conjunction with performing biopsy, endoscopy, bronchoscopy, laparoscopic surgery, or other subcutaneous or internal procedures. In some embodiments, the axial stretchability of one or more the light transducing components as increased in the length as compared to original length is 2%, 3%, 5%, 10%, 100%, some other percentage. This ability to stretch also decreases the likelihood of broken or retained component during these procedures.

In some embodiments, the light transmitting components are comprised of one or more components which has a stiffness lower than standard silica fiber optic tissues and may be optimized for a specific target procedure, tissue, organ, disease, some other use factor, or some combination thereof. Standard Fiber optics have a Young's modulus of approximately 70Gpa, which is much greater than that of healthy or diseased kidney tissue (about 30-60 kpa), liver tissue, breast tissue, gastrointestinal tissue, brain and nervous system tissue, blood vessels, and other tissues. The stiffness of the standard silica optical fibers would be especially problematic when comprising any light transducing materials extended distally beyond the needle tip, creating high risk for blunt trauma to the tissue organs even which shielding that tissue from the sharp trauma of a needle. The high Young's modulus of standard fiber optics might also transmit large blunt axial forces to high strength photopolymerized gel, which could dislodge the patch material or cause other distal blunt trauma. If the Young's modulus of the light transmitting components is much lower than normal tissues or organs, components may be at risk of deforming when exposed to arterial pressure bleeding. In some embodiments, one or more of the light transducing or waveguide components is comprised of a material which has a Young's modulus of less than 30 GPa, 20 GPa, 10 GPa, 5 GPa, 1 GPa, 500 MPa, 100 MPa, 50 MPa, 10 MPa, 500 KPa, 100 KPa, 50 KPa, 40 KPa, 30 KPa, 20 KPa, 10 KPa, 5 KPa, 3 KPa, 2 KPa, 1 KPa, 0.5 KPa, 0.1 KPa, some other level, or some combination thereof. In some embodiments, one or more of the light diffusers is comprised of a material which has a Young's modulus of less than 30 GPa, 20 GPa, 10 GPa, 5 GPa, 1 GPa, 500 MPa, 100 MPa, 50 MPa, 10 MPa, 500 KPa, 100 KPa, 50 KPa, 40 KPa, 30 KPa, 20 KPa, 10 KPa, 5 KPa, 3 KPa, 2 KPa, 1 KPa, 0.5 KPa, 0.1 KPa, some other level, or some combination thereof.

In some embodiments, one more light transducing components are comprised of the optical fibers are 1000 micrometer, 500 micometer, 250 micometers, 100 micrometers, 75 micrometers, 50 micrometers, some other size, or a combination thereof. In some embodiments, one or more of the optical fibers are the size listed above (or other optimized size) while also being biocompatible, so when used for procedures such as biopsy, the overall diameter of the components inserted subcutaneously similar to devices currently used to perform biopsies, or otherwise limits damage to target tissues or organs.

In some embodiments, the device utilizes one or more of the light transmitting materials, fibers, catheters, needles, rods, substances, coatings, diffusers, attachments, motion transmitting components, some other component, or some combination of these components to be comprised of biodegradable light transducing material. This would allow for one or more of the following benefits: improve the safety of the device or components, decrease local or remote toxicity, decrease cytotoxicity, allow for biodegradation, decreased organ or tissue disfunction, deeper or prolonger insertion or transmission within an organ or tissue, decreased likelihood mechanical injury, optimized photopolymerization, stronger hemostatic affect, other specific mechanical properties, lower regulatory barriers, reduced the cost of disposable components, increased user adoption, some other benefit, or a combination there of. In some embodiments, one or more biodegradable light transducing component are utilized. Light-transmitting biomaterials are especially important for use in some embodiments using distally extended light transmitting components such as the light transducing catheter, light rod, or other embodiments. Because all or a portion of these components are extended beyond the need or cutting portion of the device, there is significantly increased likelihood for disruption, cutting, or otherwise retained portion of the device. Creating partial polymerization of the patch material while some of that material is still within the lumen of the needle, light conducting catheter, or other component (so that the patch material can have the toughness to create a seal in the setting of arterial bleeding), also increases the advantages of embodiments comprised of biocompatible light transducing components to an unanticipated degree.

These light-transmitting biocompatible materials may be comprised of one or more types of poly L-lactic acid (PLLA), poly D L-lactic acid (PDLLA), poly lactic acid (PLA), polydimethylsiloxane (PDMS), poly octamethylene maleate citrate (POMC), Poly octamethylene citrate (POC), other citrate containing polymer, poly dimethyl-diphenyl siloxane (PMD-DPS), polyethylene glycol (PEG), Poly acrylamide-alginate (PAAm-Alg), PEGDA, APBA, Ca Alginate, cyclic olefin copolymer, conductive polyethylene, silk, cellulose, cells, bacterial cell based optical fibers, hydrogel, polycarbonate, some other biocompatible light-transmitting material, or combinations thereof.

In some embodiments, the material comprising all or a portion of the light transducing components have undergone one or more types of specific fabrication methods such as specific types of 3D printing, casting, thermal drawing, extrusion, spinning, electrospinning, some other fabrication process, or combinations thereof.

In some embodiments, the light source is connected to the needle and/or lies within the central lumen of that device, component, or needle. In some embodiments, the light source or diffuser is within the center needle. In some embodiments, the light source is able to diffuse light to either within the lumen of the needle or instrument, or external to those locations, or both of those locations. The benefits of combining both types are described earlier within this application.

### C. Optical Elements

In some embodiments, the light source is coupled to a flexible catheter which can transmit the photons near the desired target location and then diffuse them in a simple or optimized fashion to the target tissue, tract, or other locations. In some embodiments, the catheter comprises silica, fluorozirconate, fluoroaluminate, chalcogenide glasses, crystalline materials (e.g., sapphire), combinations thereof, or other suitable materials. This catheter can sit outside of the external cutting needle, between the needles, or within one of the needles. In some embodiments, the light diffusing catheter is extended to near to or beyond one or more of the inserted needle tips which allows the catheter to apply some radial force throughout the time which it is in the extended position, or alternatively it can be more flexible and only apply lateral pressure while patch material is pumped through its central lumen. This light catheter also allows the patch material to partially cure before exposure to turbulent bleeding or wet tissues, reducing the necessary viscosity and rheological requirements of the unpolymerized patch material and improving the ease of delivery. Light diffusing catheter embodiments also have the unanticipated benefit of providing radial traction on the tissues, which can decrease the extent of bleeding or collapsed surrounding tissue, allows more time and distance for photopolymerization to occur before its exposure to arterial pressed bleeding. This improves the likelihood of a strong seal so the core and/or cutting needle can be withdrawn slightly proximal too or significantly proximal to the biopsy location. Withdrawing the needle into the light diffusing catheter protects the needle from causing further trauma to the target tissue, allows for the sharp components to be retracted out of the dangerous organs or tissues, covers the vessel tips, and provides other benefits as well.

In some embodiments, photons are guided from the light source to an emitter or a target via a single fiber optic cable, a micro lens array, colloidal micro-lenses, a fused silica microarray, two types of fiber optic glass in a single mode and multimode, photonic crystal fiber for nano fiber optics, 2 and 3 photonic crystal fibers have multiple air channels that can hold substances, numerous fiber optic cables, or combinations thereof.

In some embodiments, the fiber optic nanofibers comprise portions having the diameter of light wavelengths. In some embodiments, fiber optic cables comprise tapered portions to allow for connections. In some embodiments, optical elements comprise substances treated chemical treatment such as exposure to hydrogen molecules to decrease solarization of transmission components. In some embodiments, optical elements comprise nano fiber optics with Bragg or long-period gratings, but other mode-selection schemes (i.e., photonic lanterns and patterned waveguides

In some embodiments, optical elements comprise substances that contain light diffusion enhancing nanoparticles such as aluminum, copper, gold, or silver nanoparticles. In some embodiments, optical elements comprise graphene. In some embodiments, optical elements comprise graphene-based FETs, which are considered biocompatible and chemically stable. In some embodiments, graphene functional field effect transistors are used for detecting specific proteins or substances.

In some embodiments, one or more of the materials, or a combination there which comprise the catheter material is acoustic dampening and thermally resistant. These embodiments have the advantage of being able to alter tissue to cauterize bleeding, and isolate area of tissue alteration to a portion of the distal end of the needle. In some embodiments, this material is PEEK, ULTEM, some other acoustic dampening and thermally resistance substance, or a combination there of.

In some embodiments, one or more of the portions of the device which are acoustic dampening and thermally resistant also comprise one or more portions of the device operatively connected to the wave guides, light diffusers. For example, some embodiments use acoustic cautery in combination with a catheter with embedded light waveguides, which can protect those portions of the device from heat and vibration related to high-intensity focused ultrasound (HIFU) and protect tissue from those effects as well. This allows the synergistic healing effects of isolating the target tissue, decreases local migration and embolization of the patch material, allows smaller volumes of patch material to be used while still able to polymerize in otherwise actively bleeding areas at hydrostatic pressures, and decreases the volume of HIFU altered tissue to protect remaining portions of tissue, and to protect the integrity of the waveguides.

### 1. Extendable Waveguide

In some embodiments, the device comprises a biodegradable waveguide, which makes up the entire waveguide or a portion there of In some embodiments, the device comprises a biocompatible waveguide, which makes the distal 20 cm, 15 cm, 14 cm, 13, 12, 11, 10, 9, 8, 7, 6, 5, 4, 3, 2, 1, 0.5, a different set length less than 1 cm of the waveguide, or some combination thereof. In some embodiments, the device comprises a biodegradable waveguide, which makes up the distal 20 cm, 15 cm, 14 cm, 13, 12, 11, 10, 9, 8, 7, 6, 5, 4, 3, 2, 1, 0.5, a different set length less than 1 cm of the waveguide, or some combination thereof. The portions of the needle most likely to bend during a procedure are in the distal 3-5 cm of length, which is especially true of the internal core biopsy needle during kidney, liver, prostate, solid mass, biopsies. This type of biopsy needle is weak to distal rotational and torsional forces at the biopsy trough. And bone biopsies are often affected as well. When needle bending occurs (during photopolymerized patch testing procedures), traditional glass waveguides embedded within needles have a high likelihood of fracture and subsequent loss within the biological tissue. This would require surgical retrieval to prevent secondary injury in live patients. Using biodegradable waveguides solves this problem because less likely to fracture with bending, and would not require surgical retrieval. They have high internal optical resistances however, so limiting their use to the distal portions of the device limits the total decibels of light intensity lost.

In some embodiments, the device comprises two or more types of non-silica based waveguide materials to seal surgical wounds using photopolymerizing patch material. In some embodiments, the device comprises two or more types of biodegradable waveguide material to seal surgical tracts, biopsy tracts, needle tracts, other tracts, or some combination thereof using photopolymerizing patch material. In some embodiments, the device comprises two or more types of biodegradable waveguide material to transport photons to activate photopolymerizing patch material seal biopsy wounds.

In some embodiments, the device is comprised of a light diffuse which is made of biodegradable material. In some embodiments, the biocompatible optical guide type is based on the photoinitiator type and the target spectrum of light. In some embodiments, the waveguide is comprised of a polyoctamethylene maleate citrate (POMC) core with Poly Octanediol-Co-Citrate (POC) cladding.

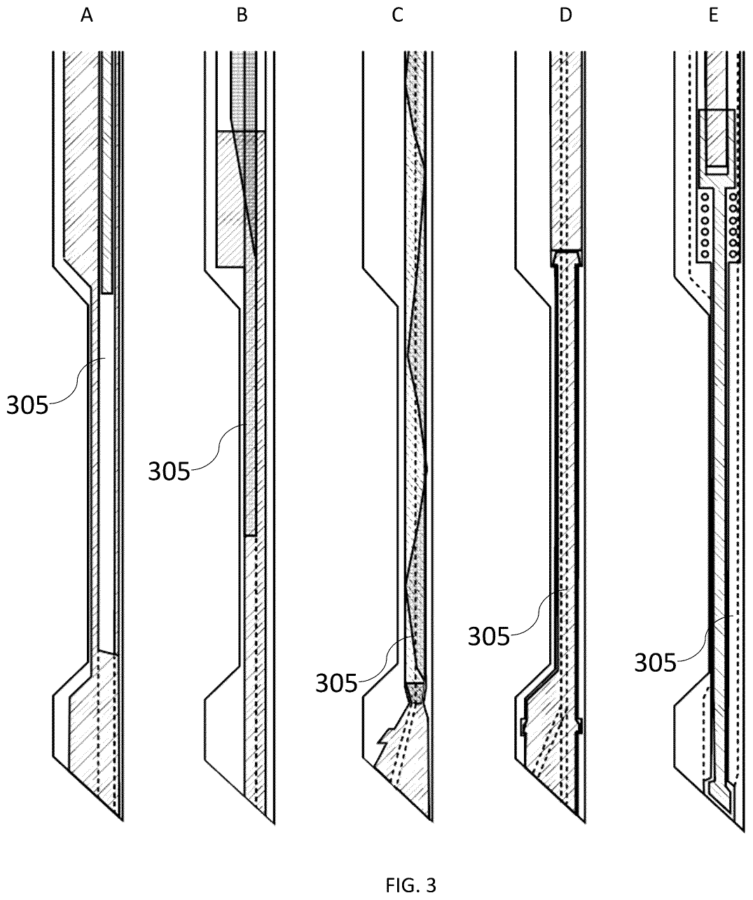

FIGS. 3A to 3E depict a biopsy needle comprising a biodegradable waveguide 305, according to some embodiments. In some embodiments the wave guide is translatable through a portion of the needle, as depicted in FIGS. 3A-3C. In some embodiments, a patch material passes around the wave guide, as depicted in FIGS. 3A and 3B. In some embodiments, waveguide is configured to emit light at more than one angle or exit bath, as depicted in FIGS. 3A, 3C, and 3D.

In some embodiments, the light source is connected to the needle and/or lies within the central lumen of that device, component, or needle. In some embodiments, the light source or diffuser is within the center needle. In some embodiments, the light source is able to diffuse light to either within the lumen of the needle or instrument, or external to those locations, or both of those locations. In some embodiments, one or more of the materials, or a combination thereof which comprise the catheter material is acoustic dampening and thermally resistant. In some embodiments, the material is PEEK, ULTEM, some other acoustic dampening and thermally resistance substance, or a combination there of.

In some embodiments, one or more of the portions of the device which are acoustic dampening and thermally resistant also comprise one or more portions of the device operatively connected to the wave guides, light diffusers. For example, some embodiments use acoustic cautery in combination with a catheter with embedded light waveguides, which can protect those portions of the device from heat and vibration related to HIFU and protect tissue from those effects as well. This allows the synergistic healing effects of isolating the target tissue, decreases local migration and embolization of the patch material, allows smaller volumes of patch material to be used while still able to polymerize in otherwise actively bleeding areas at hydrostatic pressures, and decreases the volume of HIFU altered tissue to protect remaining portions of tissue, protect the integrity of the waveguides.

In some embodiments, one or more light transducing portions of the device is thermally resistant and also HIFU resistant. In some embodiments, one or more portions or the wave guides, light diffusers, both, or a combination there of are biodegradable and have one or more portions which are covered, attached to, coated, sealed, adjacent to, otherwise associated with materials or substances which are thermally resistant and also HIFU resistant. To prevent hemorrhage in patients with advanced diseases, such as advanced kidney disease, these patients may require multiple steps or prolonged exposure of tissue sealing steps to prevent major hemorrhage. These multiple steps may cause cumulative damage to the photo transmitting portions of the device, which may increase the likelihood of breakdown, and also increase the likelihood of a portion of the device being retained in the diseased tissue. Having the combination of biodegradable photon transmitting portions along with the HIFU resistant and thermally materials, allows for longer and more intense in situ tissue sealing. In some embodiments, the device comprises an extendable waveguide for patch material activation, patch material analysis, biopsy analysis, biological substance analysis, light biopsy, sending photons from a proximal location to a distal location, receiving photons from a distal location to a proximal location, some other interaction, or a combination thereof.

In some embodiments, the extendable waveguide comprises one, two, or more mirrors, reflectors, couplers, waveguide connections, stretchable waveguide portions, telescoping waveguide sections, sliding waveguide portions, light sources, low friction components, telescoping elements, other components, or some combination thereof.

In some embodiments, the extendable waveguide is present within the main housing of the device, external to the main body of the device, within a needle cartridge of the device, within a needle, within a lumen, within an instrument cartridge of the device, driver component, actuator component, within the power unit of the device, within the light source cartridge of the device, some other location, or a combination thereof.

In some embodiments, the device comprises a rapidly extendable waveguide. In these embodiments, the device is able to cut the biopsy with at least the same distal linear cutting speed as traditional automatic biopsy device. Waveguides incorporated in rotational cutting and vacuum assisted style embodiments of this device also require waveguides that do not disrupt any slowing of a rotational speed, rotational force, distal driving speed, vacuum flow, or vacuum forces. If the mass or required drive force of the cutting portion of the device is increased as little as 15-20%, quality of biopsy samples can be significantly disrupted.

In some embodiments, the rapidly extendable waveguide comprises a coaxial telescoping mechanism. In some embodiments, the waveguide is comprised of components which are in parallel alignment of their respective axes and can slidably extend. This allows for waveguides to extend in one direction while still comprised relatively rigid structures. This also allows the unrecognized benefit of allowing use of inexpensive waveguides, such as gas or liquid surrounded by reflective cladding, to be used while still permitting extension of the waveguide length for instrumentation or patching of distal locations relative to device housing or starting position. These components would also be of great benefit to an embodiment comprised of disposable components for reducing cost, especially with use of disposable cartridges, needles, waveguides, or components.

In some embodiments, the tube cladding system providing a distal flow of gas, liquid, vacuum forces, or combinations thereof.

In some embodiments, the device is comprised of a low mass extendable waveguide. In some embodiments, the waveguide is a hollow telescoping waveguide with reduced mass relative to that of fiber optics. In some embodiments, this includes one or more hollow portions of wave guides, such that one waveguide fits into another. In some embodiments, the waveguide telescoping portions cause the device to reach the appropriate wavelength to activate photopolymerized patch material in one or more limited segments of the device.

In some embodiments, the device is comprised of extendable waveguide that has one, two, or more biodegradable photon diffusers or projectors. These may be coupled with hollow or solid telescoping sections of the device. In some embodiments with device is comprised of an extendable waveguide with variable lumen diameter, such that the light is altered to the desired wavelength activate polymerization of a patch material within some portion of the device, needle, catheter, other component, or a combination thereof. In some embodiments, the waveguide comprises components that allow extension of the waveguide in the direction of instrumentation.

In some embodiments the device comprises a low friction wave guide. In some embodiments the device comprises a telescoping waveguide that is an integral portion of one or more needle driving mechanisms. In some embodiments, a light source is integrated into an extendable portion of the device. In some embodiments, the light source or components creating or supplying energy to the light source is an integral portion of the needle driver component.

When using traditional fiber optic cable, the mass of the fiber optic or force required to straighten a curved segment can cause disruption of the speed or cutting force, thereby disrupting the biopsy cutting action. The traditional fiber optic would also take up excessive amount of space within the device, decreasing the space available for biopsy driving mechanisms. A light source or wave guide components with low mass, hollow, low friction, or other force reducing components increases the likelihood of successful biopsy. It also reduces the risk biopsy column fragmentation, and increases the width and length of biopsy column, reduces the number of required biopsies, and improves its efficiency. By integrating the waveguide or wave guide coupler into the biopsy driving mechanism, plunger, needle connector, needle axial controller, biopsy control mechanism, or biopsy needle actuator, this component provides those above benefits, but also ensures close coupling of the wave guide portions from the light source to the needle, and works synergistically with photopolymerized patch material to decrease the likelihood of major hemorrhage, by preventing macroscopic tearing of tissue (by prevention of slowing the biopsy cutting speed or reduction of cutting force) which is more difficult to seal with photopolymerized gel.

In some embodiments there are sensors integrated into the extendable waveguide. In some embodiments, light sources can be integrated into the light source.

In some embodiments the waveguide is extendable and slotted. In some embodiments, the waveguide is slotted in two or more locations. In some embodiments, there is a lateral connection between two waveguides, such that the light is transported from one segment to the other by a light bending segment of waveguide. In some embodiments, the light bending portion of the waveguide comprises a portion of the biopsy needle. In some embodiments, the light bending portion of the waveguide comprises the distal driving connection to one or more portions of a biopsy needle. In some embodiments, the extendable waveguide comprises a connection between the light source and the distal half of the needle via a lateral waveguide connector.

In some embodiments, connections between fiber optics are made from MTP (Multi-fiber Termination Push-ons), MPO fiber optic connectors, LC connectors, SC connectors, FC connectors, ST connectors, MT-RJ connectors, or combinations thereof.

In some embodiments the shape of the waveguide is circular, square, rectangular, some other shape, or contains segments of various shapes in in the cross-section. FIGS. 39A-D depict various embodiments of one or more waveguides 3905 and conduit 3910 for delivering a biocompatible patch material through a needle lumen. FIG. 39A depicts a waveguide 3905 and conduit 3910 enclosed in a circular cross-section of a lumen, according to some embodiments. FIG. 39B depicts waveguides 3905 and conduit 3910 enclosed in a circular cross-section of a lumen, according to some embodiments. FIGS. 39C and 39D depict waveguide 3905 and 3910 provided in a partial or semicircular portion of a conduit, according to some embodiments. These embodiments may correspond to the arrangement of a wave guide and conduit in sections of a lumen adjacent to a sample tray of a biopsy needle, as disclosed herein. In some embodiments, as depicted in FIG. 39C, a surface 3912 of a wave guide 3910 is exposed to allow emission of light from the surface 3912.

### 2. Emission

In some embodiments, light is emitted from one or more locations on a needle, including: the needle tip, the side of the needle, the shaft of the needle, the inside lumen of the needle, a cutting sheath of the needle, a biopsy portion of the needle, a face of an instrument facing a specific anatomical location, a surface of a vascular instrument, a surface of an introducer, a portion or all of forceps, or a combination thereof.

### 3. Fiber Optic Materials

In some embodiments, fiber optic materials comprise Erbium, Ytterbium, Erbium-ytterbium, Thulium, Neodymium, Telecom fibers, or combinations thereof. In some embodiments, optical elements comprise liquid optical fibers, optical nanowires, fiber optic lasers, silica, glass, crystal, or combinations thereof. In some embodiments, optical nanowires are configured to only allow half the light out of their sides.

### 4. Photon Wavelengths

In some embodiments, the wavelengths of photons emitted are in the UV spectrum, Visible Spectrum, Infrared Spectrum, other wavelengths, or some combination of these. In some embodiments, the UV spectrum 360 nm is used to activate the gel. In some embodiments, the far UV spectrum is utilized because it is still high energy and can quickly activate photopolymerized gels (in less than 5-10 seconds in some instances), but is known to be safe to mammalian tissues and eyes, penetrating only micrometers into biological tissue In some embodiments, the wavelength used to activate a photopolymerized patch material is between 365 nm-405 nm, 400 nm-450 nm, 385 nm-420 nm, or at times activated at a different wavelength.

### D. Fluid Circuitry

In some embodiments, the biocompatible patch based material is transmitted via pneumatic, thermal, photothermal (i.e., one wavelength expands/expels while another wavelength cures), mechanical, electrical, piezoelectric, magnetic action, capillary action, or a combination thereof. In some embodiments, there are one of more components within the device housing, body, needle, or other component or portion of the device, which can be used to advance the patch material through a small lumen of needle or other instrument despite high viscosities. These would include one or more components such as plungers, peristaltic pumps, helical pumps, gear pumps, centrifugal pumps, hydraulic mechanisms, air pumps, liquid pumps, internal or external balloons, or a combination of these components.

In some embodiments, timing and/or location release of, type and/or types of substance(s) released, transmitted to, amount of, the type, multiple types of, related features of the release of the photo activated substances may be once or multiple times; may have one, two, or more manual triggers, may have one, two, or more manual triggers; may be timed related to with a specific tissue; comprise automatic timing by the device; comprise manual timing by the device; comprise semi-automatic timing by the device; comprise timing related to the photoactivated substance ejection; comprise timing for immediate release of photons; comprise timing for delayed release of photons; and sometimes the photons are not released at all

In some embodiments, release of a patch material is timed before, during, or after a procedure is completed, or some combination thereof; with timing before, during, or after a needle is inserted, or some combination thereof; with timing before, during, or after a tubular structure is entered or some combination thereof; a needle and/or instrument segment entering an organ, certain portions of an organ, a tumor, certain portions of a tumor, and/or biological structure; a needle entering the skin; a heating process taking place; when freezing takes place; with timing before, during, and/or after a biopsy is performed; with timing before, during, or after a substance is released from the needle or instrument or some combination; with timing before, during, or after surrounding organs, portions of organs, vessels tissues, biological structures, substances, distance from points with a body, with timing to related input measuring to the extent to which a chemical, physical, biological, thermal, cooling, electrical, or photon activated process has occurred; or some combination thereof. For example, in some embodiments, the extent to which a substance has undergone-photochemical activation can be detected using optical input examining the target region, and the device can notify the user of that progress, and/or make computer based and/or computer aided decisions as to when, and/or how long photons will be emitted from the device. In some embodiments, the fluid channels for delivering the patch material are configured to reduce turbulent flow. In some embodiments, the final portion of the patch material emitted is void of a photoinitator to prevent unwanted adhesion, for example, to the device.

In some embodiments, timing is relative to sensor related input measuring to the extent to which a chemical, physical, biological, thermal, cooling, electrical, or photon activated process has occurred. For example, in some embodiments, the extent to which a substance has undergone-photochemical activation can be detected using optical input examining the target region, and the device can notify the user of that progress, and/or make computer based and/or computer aided decisions as to when, and/or how long photons will be emitted from the device.

With timing related to the shape, structure, stricture, internal flow, internal pressures, luminal wall pressures, hemorrhage, oncotic pressures, physiology, pathophysiology, damage to, missing, removed, excised, manipulated, added, repaired, surgical portion and/or other features of a tubular body that have been and/or will be sensed, detected, calculated, and/or proposed by the user and/or computer software

In some embodiments, timing is relative to the shape, structure, stricture, internal flow, internal pressures, luminal wall pressures, hemorrhage, oncotic pressures, physiology, pathophysiology, damage to, missing, removed, excised, manipulated, added, repaired, surgical portion and/or other features of portion of an organ which has been sensed, detected, calculated, and/or proposed by the user and/or software.

In some embodiments, timing is relative to the shape, structure, stricture, internal flow, internal pressures, luminal wall pressures, hemorrhage, oncotic pressures, physiology, pathophysiology, damage to, missing, removed, excised, manipulated, added, repaired, surgical portion and/or other features of an organ sensed, detected, calculated, and/or proposed by the user and/or computer software.

In some embodiments, the device is configured to prevent a photoactivated substance from adhering to the device. In some embodiments, photons received from tissue to device are used to determine one or more diagnosis, prognosis, severity of disease, guide recommended treatments, response to treatment, need to change medication or treatment, and/or determine type, intensity of, duration of, and/or factors of sealing tissue and/or tract.

In some embodiments the area of photo-activation is offset away from the device instrument, housing, and/or needle by some distance. Such as two photo activation at certain distance from the instrument. This would allow for sealing tissue without sealing the instrument itself to the photoactivated substance. In some embodiments the device could use this feature to create a precise 3D print inside and/or on the surface of biological tissue and/or structures. Such as repairing or stenting a vessel, repairing and or replacing structures in the ear, replacing portions and mass of biopsied tissues.

In some embodiments, timing is relative insertion of the needle into biological tissue, retraction of the needle from some portion of and/or all biological tissue, on interaction with a specific tissue, or a combination thereof.

### 1. Multi Driver System

In some embodiments, the device couples the activation of patch material delivery, tissue alteration, or both to the activation of other portions of the device. In some embodiments, this includes coupling the injections and activation of the patch material to one, two, three, or more drivers. These drivers can be the driving motion steps of biopsy such as that in an automatic core biopsy device. For example, in some embodiments, the user may trigger the firing of biopsy which may cause a first driver spring to be activated causing the internal needle to be advanced to a target position. As this first spring approaches full extension, it can activate a second driver spring as it pushes retain portions free from their hold. There is an additional coupling in some embodiments, such that can be coupled with every activation, or toggled on or off by the user as shown in the associated images. When the patch material delivery coupling mechanism is toggled to the on position (or permanently unlocked), the activation cascade caused by the biopsy activation can trigger one or more of the following actions: mixing of patch material, activation of patch material within some portion of the device, the release of patch material, the activation of patch material some location outside of the device, the advance of some portion of the device (such as a plunger), the rotation of some device (such as a pump mechanism), electrical activation of a lighting system, electrical activation of RF cautery, electrical activation of HF ultrasonic cautery, retraction of a needle, retraction of sheath, retraction of a catheter, retraction of some other portion of the device, advancement of some portion of the device, the ablative step, or some combination there off. This allows the user to rapidly activate the patch delivery and activation mechanisms to ensure the portion of the tissue injured by the biopsy (or related procedure are closely coupled. This is especially beneficial to preventing arterial pressure bleeding related to biopsy, making precise and repeatable results of mixing and partially polymerizing the patch material within some portion of the device. In some embodiments, triple driver is coupled to the retraction of one or more elements by the third driver in the system, and this retraction motion may cause mechanically the injection and activation of patch material, advancing a plunger or causing rotation of a pump component. In some embodiments, the coupling mechanisms and components between each of the drivers can comprise one or more: mechanical displacement of retaining elements, axial motion, rotational motion, vacuum pressure, motion outside of the plane of advancement, electrical activation, magnetic activation, piezoelectric activation, mechanoelectrical, or some combination thereof. In some embodiments, the drivers are comprised of one or more springs, electrical motors, piezoelectric motors, other stored mechanical energy, other stored electrical energy, other driver, or some combination thereof.

In some embodiments, one or more of the triple driver system is mechanically coupled to a cocking (resetting system). In some embodiments, the resetting system reloads the patch material within the device, reprimes the patch material within the device, resets the needles from a retracted position, resets a needle from a distal position to a “read position”, activates an ablative step, ejects a portion of the device, reveals the biopsy specimen the user, restores an electoral charge in a battery, restores an electrical charge to a capacitor, or some combination thereof.

In some embodiments, there is a preferred sequence of events for the triple driver activation and reset for use with a single hand cocking and activation. In some embodiments, activation causes full extension of the internal biopsy needle, the first driver then activated the second on full extension, thereby causing the external biopsy needle to advance and cut the biopsy sample to be cut from the target tissue. This second driver then causes activation of the third driver, which simultaneously causes the retraction of both needles in unison from the target site at a controlled rate as the patch material delivery system advances and activates the patch material. This activation of patch material can occur within a portion of the device such as within the bore of the needle, within a catheter, outside a portion of the device, or multiple locations. This combination is preferable for devices intended for biopsy and sealing tissue using photopolymerized patch material with strongly adhesive forces exceeding that of hypertensive arterial pressure bleeding. The cocking steps coupled to this example triple driver system, in some embodiments, also has a cocking sequence that can be set with one hand using a cocking lever system and resetting mechanisms. In some embodiments, once withdrawn from the patient, the user can pull the lever once to return the two needles to the un-retracted position which also resets/reprimes the biopsy needle with patch material. The second activation of the cocking lever then retracts the external needle, thereby revealing the sample in the inner needle trough. The third activation of the cocking lever then returns the internal needle to the initial position and readies the activation button for use.

Various other configurations and timing exist in some embodiments. For example, in use for breast biopsy, the device may only have one needle driving mechanism followed by a sample vacuum mechanism. In such cases, it may be preferred to deliver the patch material using the rotational cutting action to deliver the patch material, such as an internally rotating body that retracts the internal needle and biopsy sample on terminally distal location, revealing a low profile waveguide and rotational patch pump.

### 2. Controls of Patch Delivery Mechanism

In some embodiments, the device comprises a system for the user to control the release of patch material so as to toggle it on, off, increase duration, decrease duration, other modifications, or a combination there of This can be important as the user may not want to use the patch material process for every pass of the instrument. This may be important because the user may want to only seal the common biopsy tract formed on the final pass. When using the single or limited patch dose cartridges, this may be of unanticipated benefit of getting more biopsies performed using a single cartridge.

In some embodiments, the device is comprised of a limited number of patch doses. In some embodiments, the patch material can only be used to seal the needle tract of one pass, two passes, or some other limited number of needle passes or instrumentations. In some embodiments, the patch material can only be used to seal the needle tract of one biopsy, two biopsies, or some other limited number of needle biopsies. In some embodiments, toggling on, off, other changes to the patch material release/activation, or some combination thereof, is controlled by a switch, nob, roller, dial, button, touch screen, sensors, computer process, output, automatically controlled, semiautomatically controlled, remotely controlled, other mechanism, or a combination there of In some embodiments, these inputs may be used to control the mixing, pumping, extruding, activating, other mechanism related to the patch material, or a combination thereof. This modification of timing and sequencing is also of benefit to users who do not wish to use the patch functions on every pass of the needle.

In some embodiments, the device comprises a physical mechanism to couple, decouple, otherwise modify, or a combination thereof the interaction the patch release and curing from the other mechanism of the device. In some embodiments, the device comprises a physical mechanism to couple, decouple, otherwise modify the interaction of, or some combination thereof the patch release and curing from the biopsy action of the device. In some embodiments, the device is comprised of this action is a physical mechanism causing a change, such as taking two gears out of alignment, modifying the alignment of a post and rotational guide, separating a key and keyhole, causing a rotational or translational movement of one or more components relative to one or more other components, separating a rider and tract, separating a wheel and tract, change in an actuator position, piezoelectric change, some other mechanical mechanism, some other switch, or a combination there of This change can be electrical, sensor based, automated, semiautomated, physical, other changes, or a combination there of.

In some embodiments, the device comprises a control to reset the ability for the device to perform another procedure. In some embodiments this control is automatic, while in others there is a lever, button, handle, crank, switch, a combination of these or some other means to reset the device for activation. In some embodiments of this device, the cocking mechanism is coupled mechanically, electrically, or otherwise, to the initial priming or repriming of the patch material system such that the patch material system is also made ready by the action. Alternatively in some embodiments, priming the device for use with the patch material also resets the driving mechanism, thereby cocking the device. In some embodiments, the action of loading or unloading of a needle, instrument cartridge, patch cartridge, light cartridge, some other component, or a combination thereof into a portion of the device activates the cocking mechanism, preparing the drive actions for use

In some embodiments, the drive mechanism also generates the electrical, mechanical, electromechanical, piezoelectric, magnetic, or elastic energy to power the light source of the device. This may reduce the need for recharging, or enhance performance in austere conditions.

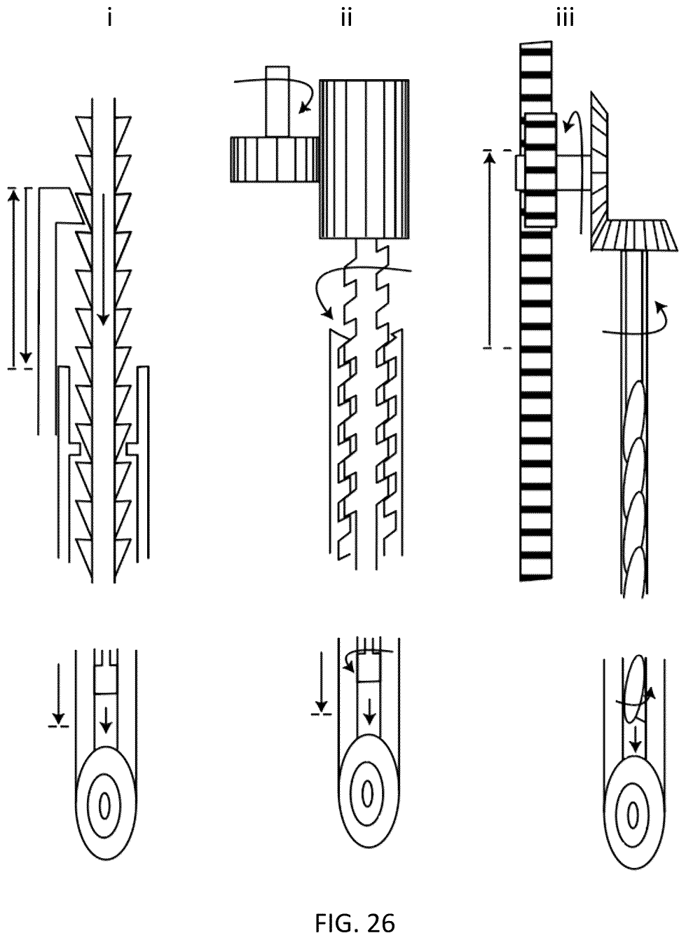

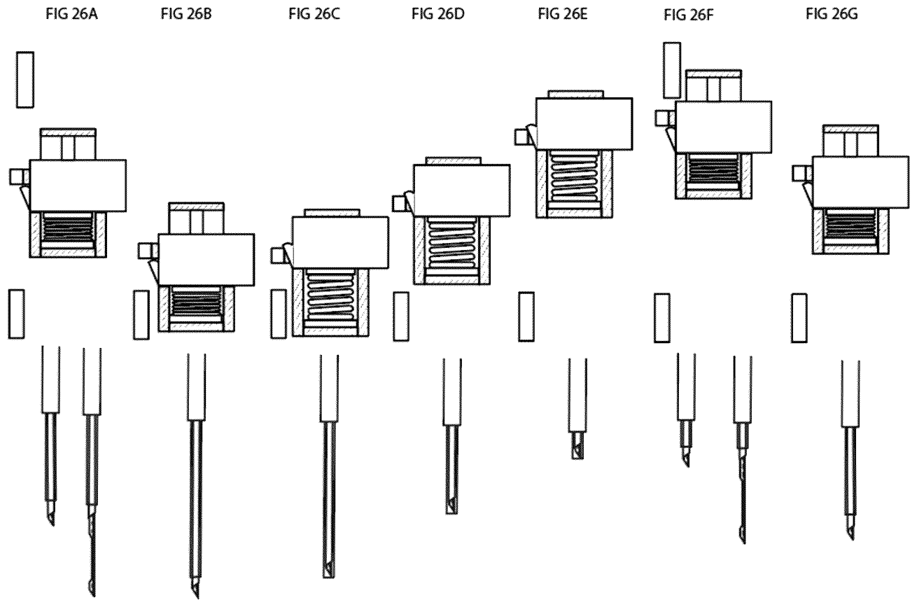

FIGS. 26A-34 depict various embodiments of fluid components for controlling delivery of patch material, according to some embodiments. Each of which will be recognized as having an advantage for incremental dispensing of a patch material.

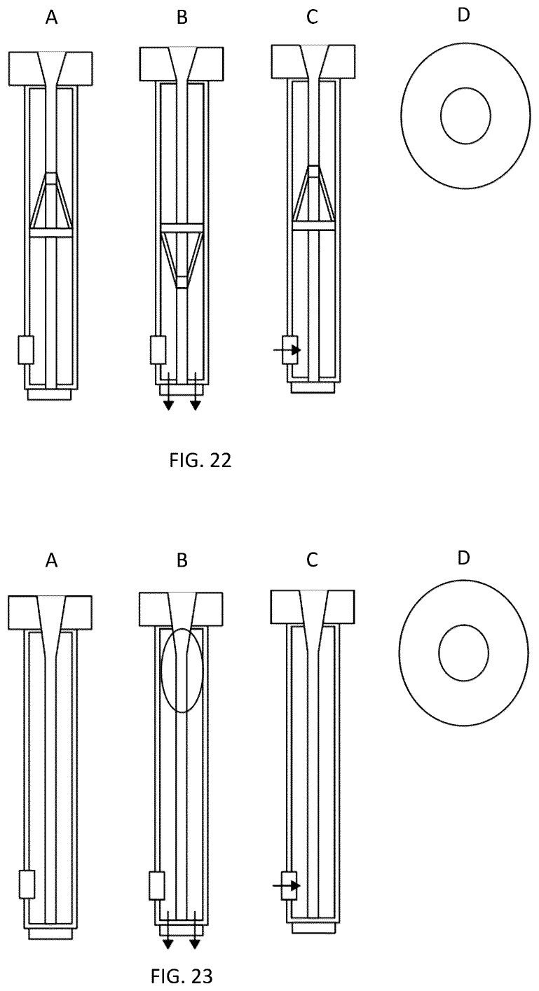

FIGS. 22A-22D depict a plunger which translates to force patch material from an outer lumen of a fluid delivery component. FIGS. 22A-23D may utilizes expansive properties of substances under heat to drive a fluid system.

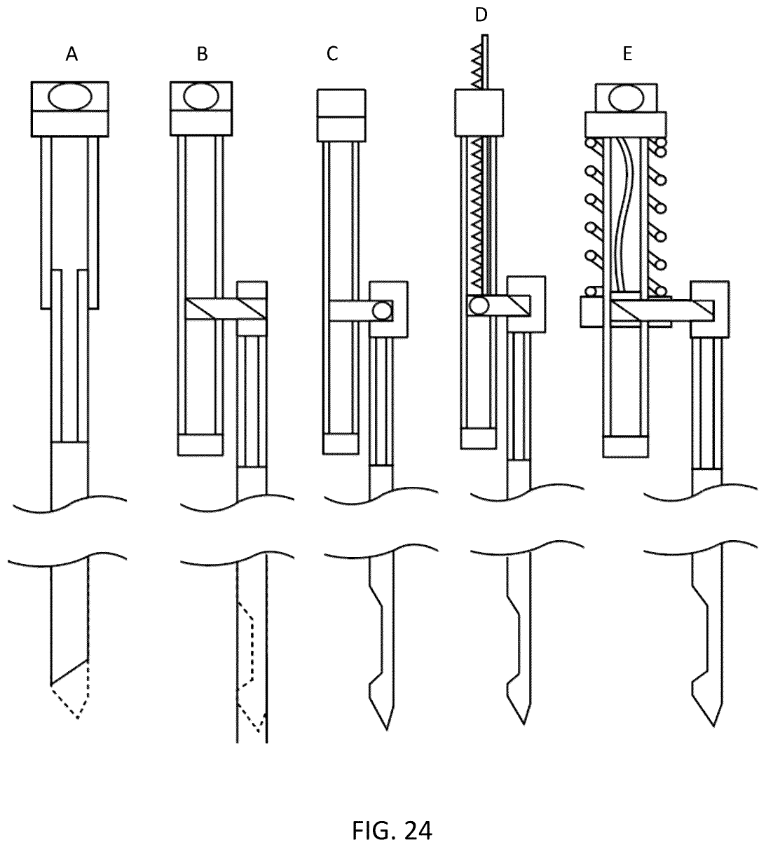

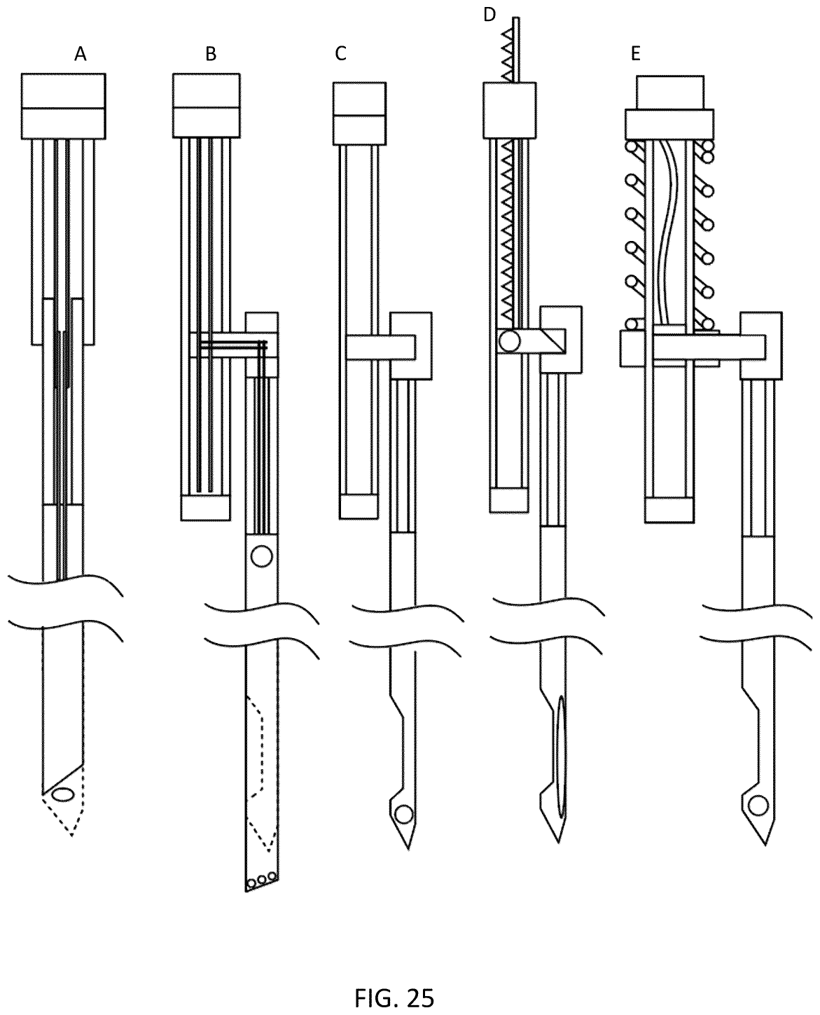

FIGS. 24A-25E depict various embodiments of vertically translated systems to translate patch material and/or components of a biopsy needle.

FIG. 26A depicts a fluid drive system with locking groves to prevent the drive mechanism from back up due to back pressure.

FIGS. 26B-26C depict a fluid drive system wherein rotation of components is translated to vertical motion of a fluid.

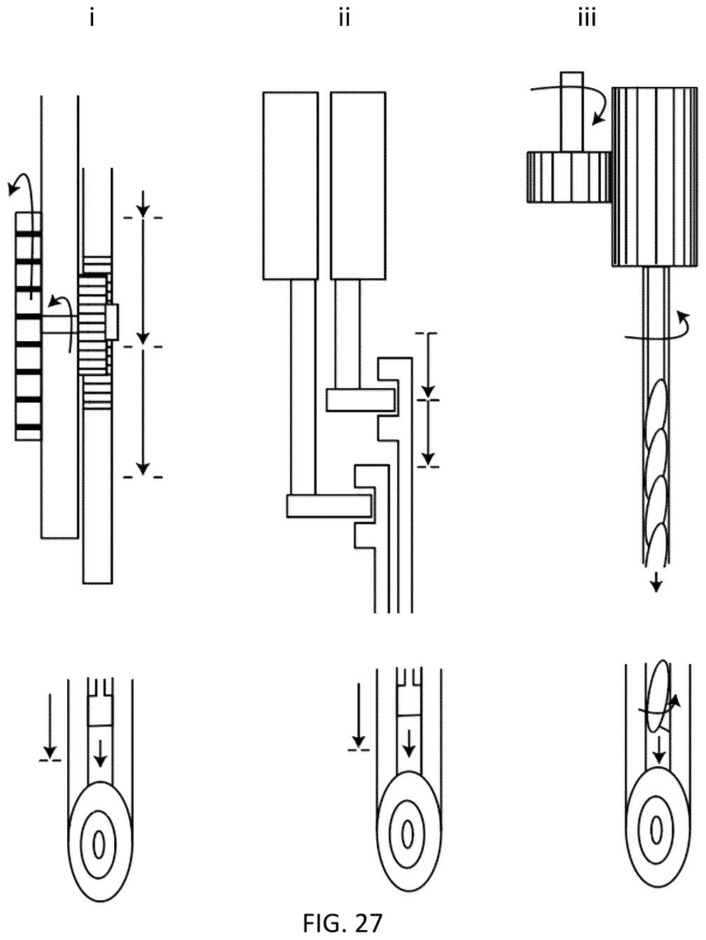

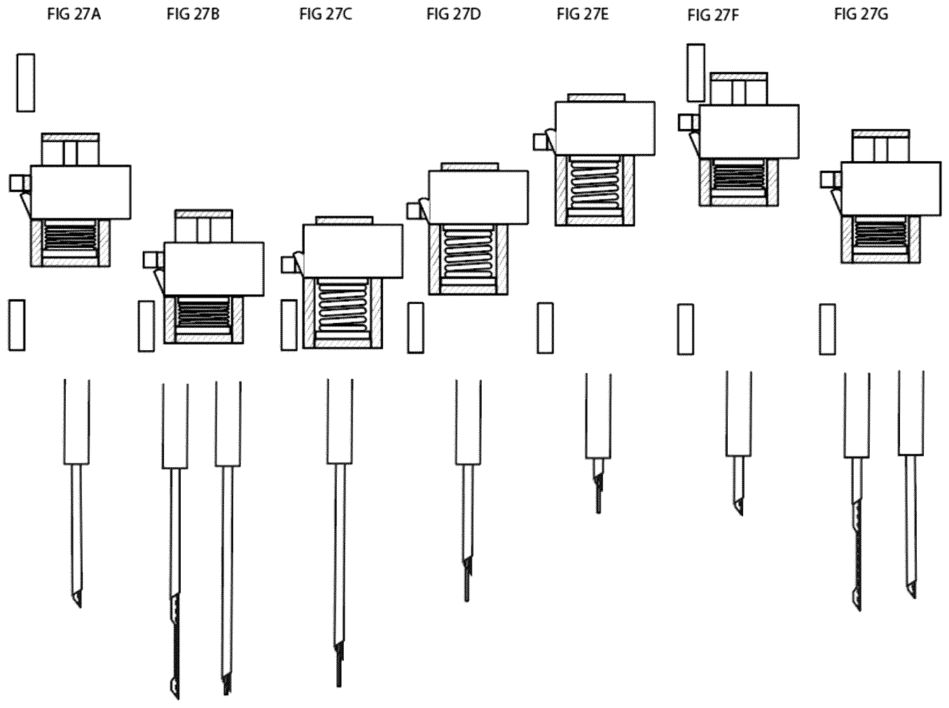

FIG. 27A depicts a rotating gear and corresponding rack system to drive a patch material from an end of a lumen.

FIG. 27B depicts a dual translation system to drive a patch material more than one patch material from an end of a lumen.

FIG. 27A depicts a rotating gear system to drive a patch material from an end of a lumen.

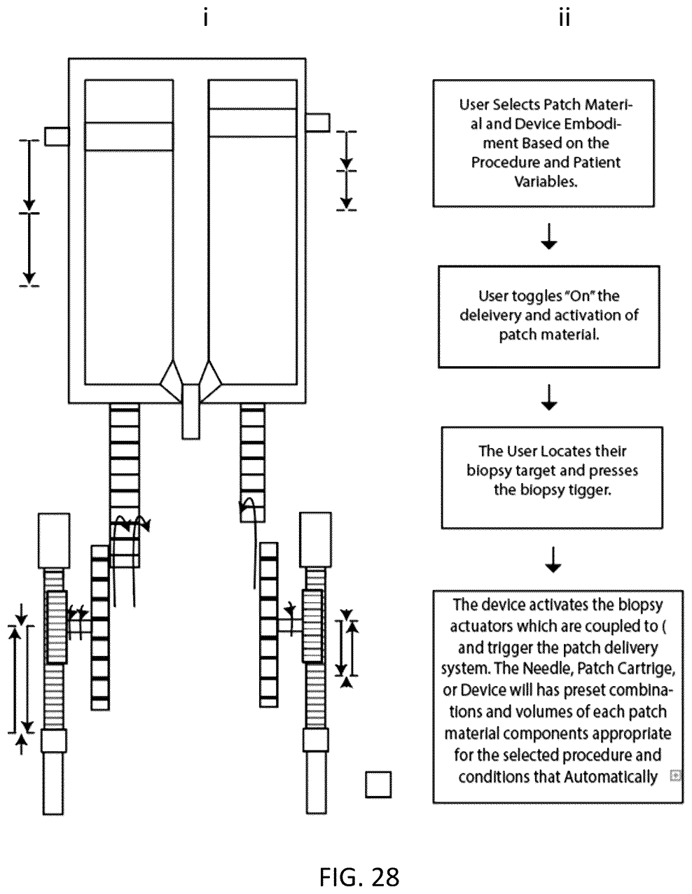

FIGS. 28A-28 B depict a method for delivering more than one patch material, wherein delivery of each patch material may be controlled separately.

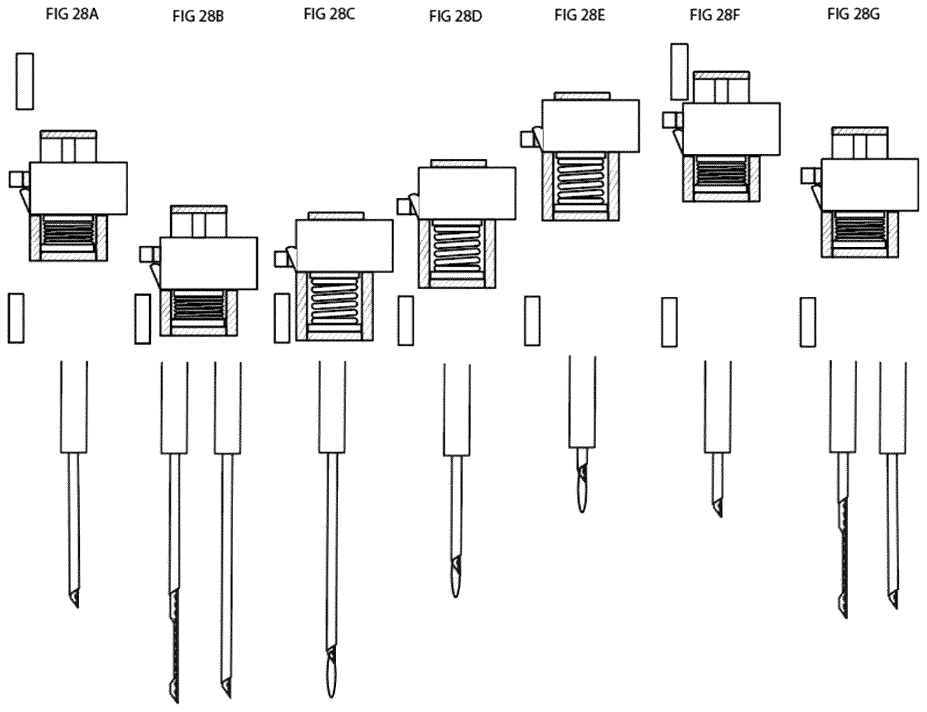

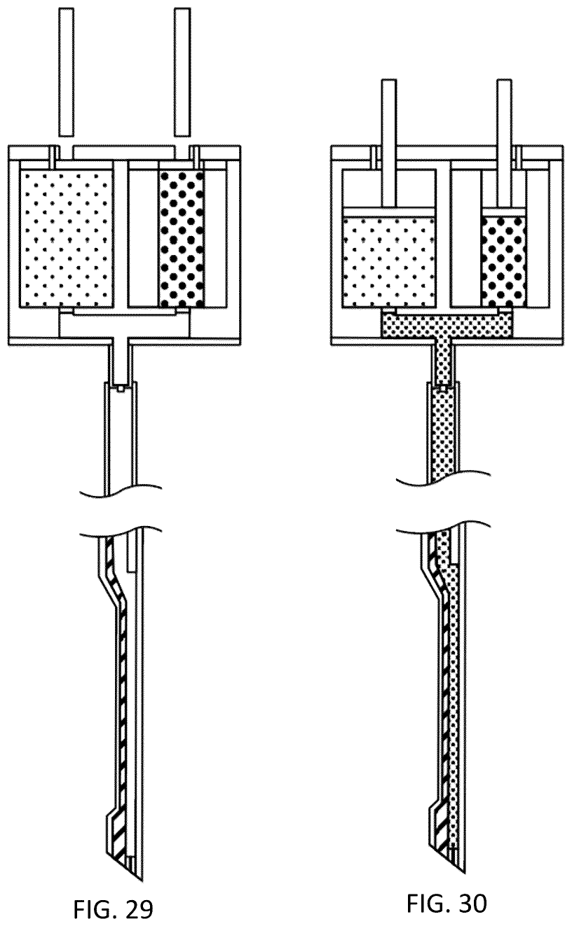

FIGS. 29-30 depict a system for delivering different concentrations of different patch material through a shared lumen. In some embodiments, internal features are added to insure thorough mixing of the patch materials.

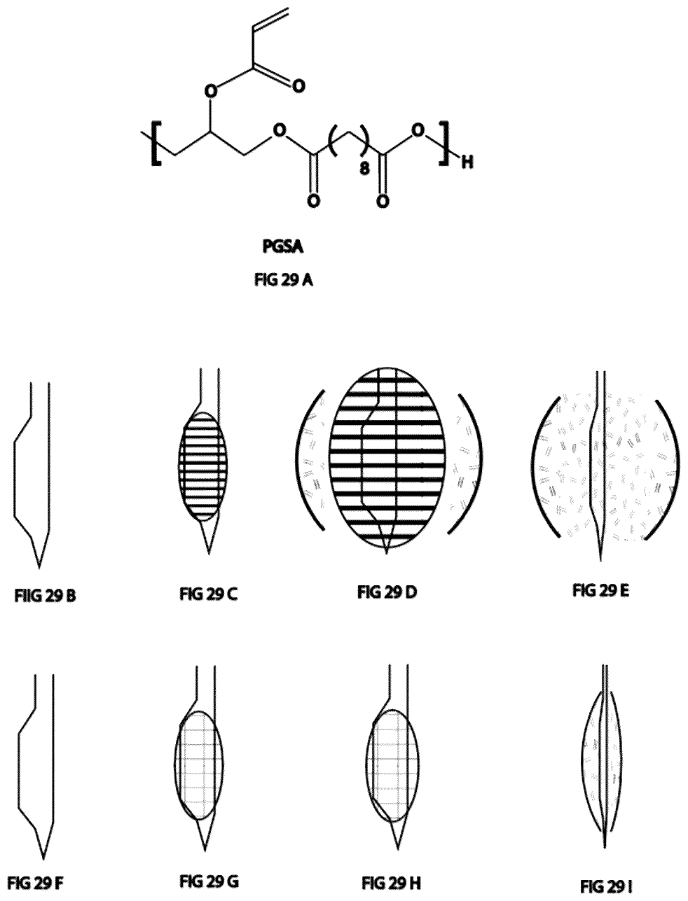

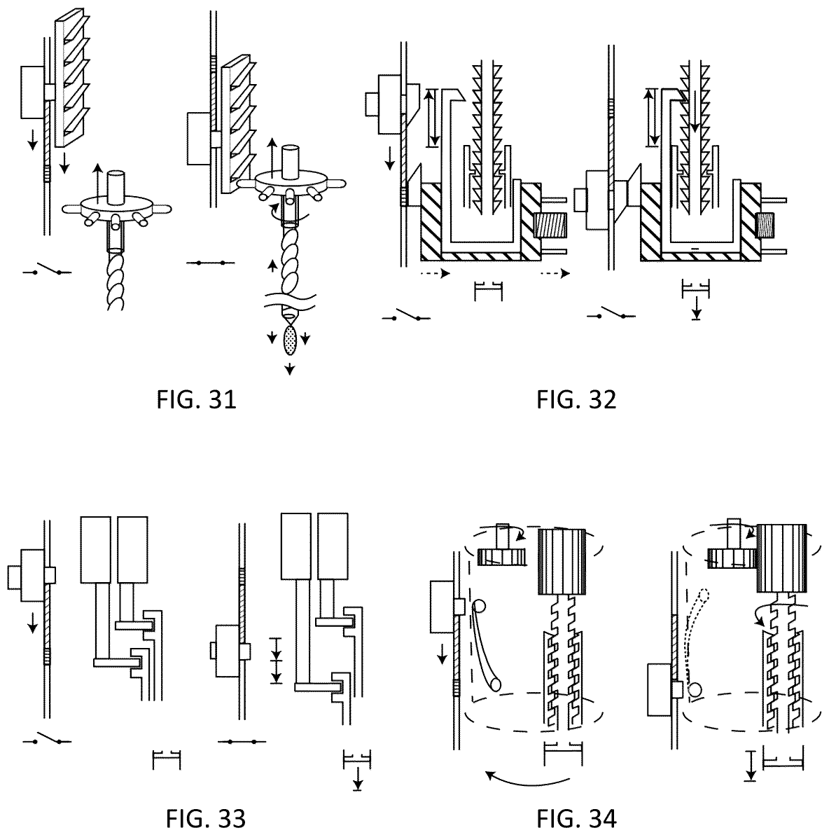

FIG. 31 depicts a system for delivering a patch material in increments utilizing a sliding rack which engages in a rotatable wheel.

FIG. 32 depicts a system for delivering a patch material in increments utilizing a locking sliding rack.

FIG. 33 depicts a system for delivering a patch material in increments utilizing a dual translating actuators.

FIG. 34 depicts a system for delivering a patch material in increments utilizing a rotating gear system which is selectively disengageable.

### 3. Timed Retraction

In some embodiments, one or more portions of the device comprise a timed retraction mechanism. In some embodiments, the timed retraction mechanism comprises one or more components used to optimize the seal strength, flexibility, tensile strength, constitution, young's modulus, curing, dispensing, mixing, formation rate, some other aspect of a photopolymerized patch material, or a combination thereof. In some embodiments, the timed retraction mechanism is used to optimize the seal, curing, dispensing, or some other aspect of a patch material. In some embodiments, the timed retraction mechanism is used to optimize the activation of tissue alteration along with patch material sealing. In some embodiments, the patch material cartridge attached to the device automatically adjusts the timing mechanism. In some embodiments, the power cartridge attached to the device automatically adjusts the timing mechanism. In some embodiments, the instrument cartridge attached to the device automatically adjusts the timing mechanism. In some embodiments, device is comprised of a sensing component of the device which is monitoring sealing of the curing aspect within some portion of the device which alters the timing mechanism through a central feedback system. In some embodiments, there is a sensing component of the device which is monitoring sealing of the curing aspect within some portion of the device.

In some embodiments, the viscosity of the patch material resists the force of a plunger, pump, helical pump, fluid, air, propeller, some other component, or a combination of these, which slows the mechanical retraction distal tip of the device while the patch material is polymerized and extruded from a portion of the device, thereby optimizing curing light exposure time.

In some embodiments, the device comprises a mechanism where one, two, or more catheters is retracted slower than one, two, or more needle while patch material is partially or fully polymerized within the lumen of the catheter. This allows for patch formation, lengthening, thickening, strengthening, and propagation while still within the lumen of the device. This allows for contiguous photopolymerized patch column formation even in a totally liquid environment distal to the end of the distal aperture of the catheter and/or needle. Those benefits would be especially useful when patching the biopsy of a kidney, liver, prostate, breast, brain, or within the GI tract, as this mechanism can be used to form distal patch to seal a vessel, vein, artery, AV fistula, pseudoaneurysm, meninges, or other fluid containing body entered by the distal tip of the vessel. This mechanism may be used to start the patch material thereby creating a distal anchor, or it can be used during all of a portion of the patch curing process, which would help create a solid column of patch material during all or a portion of the biopsy tract.

Alternative timing combinations creating the same benefits would include the following variations: In some embodiments, the device comprises a mechanism where one, two, or more needles is retracted slower than one, two, or more catheters while patch material is partially or fully polymerized within the lumen of the, beyond an external aperture, or both. In some embodiments, the device comprises a method where one, two, or more catheters is retracted while patch material is polymerized within the lumen of the catheter, distal to an external aperture, or both. In some embodiments, the device comprises a method where one, two, or more needles is retracted while patch material is polymerized within the lumen of the needle before being expelled from the distal tip.

In some embodiments, the device uses a mechanical component to create the timed (or prolonged) retraction, while also generating electricity. This can be especially help in locations throughout the world that do not have.

In some embodiments, the device comprises a system for the user can control the timing mechanism to toggle it on, off, increase duration, decrease duration, other modifications, or a combination there of to alter the timing mechanism. This can be important as the user may use the same device to create different effects with the patch material such as hemostasis vs. tissue sealing, may be controlled to optimize the timing mechanism for a specific organ, anatomy, disease state, electronic display, sensor reading, computation, output, other reasons, or a combination there of In some embodiments, alterations of the timing mechanism of the device are controlled by a switch, nob, roller, dial, button, touch screen, sensors, computer process, output, automatically controlled, semiautomatically controlled, remotely controlled, other mechanism, or a combination there off. In some embodiments, these may be used to prevent a mechanism from taking place, essentially turning off, on, or otherwise modifying some mechanism of the device. This modification of timing and sequencing is also of benefit to users who do not wish to use the patch functions on every pass of the needle. This can have the unanticipated benefit of preventing altering the smooth biopsy action of a subsequent biopsy cut which may occur after photopolymerization as some macroscopic or microscopic portion of patch material may adhere to a portion of the biopsy device.

In some embodiments, the device comprises a system for the user can control the timing mechanism such that one or more components are withdrawal relative to the surface of a biological tissue during advanced stages of kidney disease. This allows the patch material to gain increased tensile strength to better match that of fibrotic kidney tissue and better resist hypertensive arterial force bleeding. It also allows for more polymerization time within some lumen of the device before exposing it to tissue to make up for the lack of innate hemostatic mechanisms and increased vascular rigidity in advanced kidney disease.

### 4. Patch Priming Step

In some embodiments, the device comprises a priming mechanism to prepare the device for use. In some embodiments, the device is composed of components used to prepare the patch material for injection. In some embodiments, the priming step comprises one or more components the mixing of the patch material, movement of the patch material to the distal portions of the device, sensors to check the constitution of the patch material, plunger movement to a set location, patch material pump movement, light intensity check, light wavelength check, valve modifications, filter modifications, other patch material components, change in the indicator display, change visible in the patch material window, or some combination thereof.

In some embodiments, the patch priming step comprises the activation of one or more light sources within the device, partial polymerization of patch material, full polymerization of patch material, other patch material component alteration, or some combination thereof. This allows for patch material to better seal in the setting of hypertensive arterial pressure bleeding, organ disease preventing the normal hemostasis processes. In some embodiments, the patch priming step comprises a temperature change to one or more of the patch material components by heating, cooling, normalizing, or some combination thereof.

In some embodiments, the device comprises a patch priming step that is specific to one or more issues related to the intended patient, organ, disease etiology, disease severity, disease progression, hemophilia, coagulation disorders, platelet disorders, anticoagulation medication use, antiplatelet medication use, other medication use, Platelet Time test, International Normalized Ratio, some other measure, or some combination thereof.

In some embodiments, the device comprises one or more controls the user can adjust to select an optimized patch priming step action. In some embodiments, the device comprises simplified patch priming step selection confined to one or more simplified selection options. These selection options may comprise one or more numerical selection, qualitative selections, organ selection, disease selection, coagulation selection, blood pressure selection, platelet disorder selection, or some combination thereof.

In some embodiments, the device comprises one or more automatically controlled adjustments to select an optimized patch priming step action. These automatically controlled adjustments to optimize patch priming step action can be comprised of outputs triggered by sensors detecting changes within patch material, power cartridge selection, patch material cartridge selection, light source selection, light source sensors, computer based calculations, external sensors, internal sensors, component positions, user control positions, detected aspects of the patient by sensors, detected aspects of the biological tissue by the imaging device, the guidance system, some other component, or some combination thereof.

In some embodiments, the device comprises a patch priming step that can be activated by the user at one or more times before the procedure, after attaching a module, during a procedure, following a procedure, or some combination thereof. In some embodiments, the patch prime step can be manually activated by the user to prepare for seal after biopsy but total device withdrawal. This allows the user to selectively prime the device after biopsy so the user can prevent alteration of the normal biopsy process and mechanism, which may otherwise be altered by priming or partially polymerizing patch material.

### 5. Ablative Step

In some embodiments of the device, the device is comprised of one or more ablative steps used to help clear debris from portions of the device during use. In some embodiments of the device, the device is comprised one or more ablative steps during an instrumentation procedure and patch placement. In some embodiments of the device, the device is comprised one or more ablative steps after a cautery procedure and wound patch placement. In some embodiments of the device, the device is comprised one or more ablative steps after a biopsy sampling and wound patch placement. In some embodiments, this ablative step may be used one or more times while a portion of the device is still within tissues, within other biological substances, before use, during use, after use, in between uses, or some combination thereof. In some embodiments, the ablative step uses photons of one or more frequencies to cleave chemical bonds, mechanical bonds, physical bonds, other bonds, or some combination thereof. In some embodiments, the ablative step is used to help clean debris from one or more needle, biopsy needle, internal biopsy needle, external biopsy needle, light diffuser, waveguide, catheter, sheath, lumen, interior, exterior, coating, tissue, substance, other target, or some combination thereof. The ablative step can be performed in some embodiments by one or more methods substance ablation such as photoablation, heating, vibrating, RF electrosurgery, HIFU, Thermal cautery, freezing, some other energy transmission, mechanical brushing, mechanical sweeping, some mechanical other method, or a combination there of In some embodiments, the ablative step can be used to free one or more aspects of the distal portion of the device from the sealed patch material or tissue. It can alternatively be used to prevent debris from obstructing patch material release or mechanical function during the next procedure. It can alternatively be used to prolong the lifespan of the device, or prevent contamination of the current or subsequent biopsy samples. This step can also be used to prevent mechanical friction forces on subsequent procedures related to patch material presence. This can be helpful to preventing frictional forces from patch material even when it is present in concentrations below those visible to the human eye. It can alternatively be used to prevent contamination of a biopsy sample which has already been removed from tissue and remains within some portion of the device before the ablative step.

When used after a core biopsy procedure and patch material placement performed by some embodiments of the device, the ablative step can be used to help remove patch material from portions of the external biopsy needle, inner needle, other portions, or a combination thereof. When used after a core biopsy procedure and photopolymerized patch material placement performed by some embodiments of the device, the ablative step can be used to help remove patch material from portions of the external biopsy needle, inner needle, other portions, or a combination thereof. Most core biopsy procedures are semi-automated with mechanical driving components such as springs to power biopsy sampling. When activated, these springs create forces which drive a consistent speed of forward axial sliding translation of one needle over another to cut the tissue. But any increase in frictional forces between those needles causes an exponential increase in biopsy procedural failures. When that biopsy trough is then opened to retrieve the biopsy sample after an ablative step, there is significantly reduced risk of microscopic patch material contamination of that biopsy sample. In some embodiments, the ablative step can used after one or more biopsy procedures utilizing a rotating biopsy needle. This prevents increased friction of the rotating needle from even microscopic portions of patch material which can disrupt biopsy samples, cause injury to tissue or biopsy because of lower slicing speeds, prevents contamination of biopsy sampling, and prevent obstruction of biopsy sampling return tubing. In some embodiments, the ablative step can be used after one or more passes of a vacuum assisted biopsy procedures. can also be used to sterilize some portion of device, tissue, wound, biological substance, or some other substance to decrease the likelihood of infection related to a procedure. In some embodiments, the ablative step can be used for more than one purpose, such as both for removing patch

In some embodiments, one or more aspects of the ablative step is selected by the user from a button, dial, other control, or a combination of these things. In some embodiments, one or more aspects of the ablative step is controlled semiautomatically or automatically. These semiautomatically or automatically controls may be comprised of one or more sensor, timer, mechanical component, electrical component, computer component, processor, other external component, other internal component, or a combination there of The aspects of ablative step controlled or modulated may include one or more type of ablation activation, wavelength, frequency, intensity, onset, duration, location of device, location of tissue or substance target, some other controlled aspect, or a combination there of.

In some embodiments, the device has an automatic ablative step that is optimized for use with a specific type of patch material. Those aspects of the ablative step may be controlled by the user, semiautomatically, automatically, or a combination of these controls. In some embodiments, one or more aspects of the ablative step is controlled by the attachment of a certain cartridge or disposable component to another aspect of the device. For example, a portion of a patch material containing cartridge used for a kidney or liver biopsy (such as an external RF code or specific component shape or length) may encode specific instructions for the ablative step. These instructions may then be sensed, interpreted, and executed by other components of the device when combined. In some embodiments, when the device is activated by the user to perform biopsy while the patch material is set to seal, the ablate step is optimized to break chemical bonds of polymerized patch material at or near the surface of one or more photon diffuser components.

In some embodiments the device performs an ablative step after each use. In some embodiments, the device uses sensors (such as those used in conjunction with reflected light spectroscopy) or other components to measure the photocuring process, other patch curing process, photoablation process, some other process, or some combination of these to help control one or more aspect of the ablative process.

In some embodiments, the ablative step is used in conjunction with partial or complete activation of patch material within one or more portions of the device. Partial activation of photopolymerized patch material within a portion of the device has not previously been described for these purposes, likely because it has the tendency to obstruct these components and adhere to the lumen walls of a needle or catheter. This ablative step can clear these internal components or lumens, to allow patch material to flow with less force or debris contamination. To fully seal tissue to a high tensile strength in the setting of active bleeding, (using photopolymerized or other patch material) there is also a prolonged ejection and/or activation time, which tends to increase the amount or intensity of patch material and tissue sealed to the device components. This ablative step can decrease traumatic forces to tissues and cured patch material by better releasing the device in those settings. In some embodiments, the use of photon ablation in conjunction with photopolymerized patch material to seal wounds related to biopsy allows further unanticipated simplification, safety, and precise control of the afore mentioned benefits. In some embodiments, there are one or more ablative steps during the automated retraction process.

In some embodiments, the device is comprised of a catheter that extends over some portion of the device after one or more patch material applications to break the at least some portion of the chemical bonds of the patch material. In some embodiments, the device is comprised of a catheter that extends over some portion of the device after one or more patch material applications to break the at least some portion of the physical bonds of the patch material. In some embodiments, this ablative catheter comprises a catheter with a light diffuser in some portion of the catheter which can deliver light or more instruments within the lumen of that catheter. In some embodiments, this ablative catheter comprises a catheter with a light diffuser which projects light only within the lumen of the catheter. In some embodiments, the ablative catheter comprises a catheter used at some time periods to deliver light to cure photopolymerized patch material and at other time periods to deliver light to break chemical bonds within the cured patch material. The light for these different functions may be delivered may be from the same or different portions of the catheter, and may use the same or different wavelengths. For example, the device can comprise a light delivering catheter used during a kidney biopsy, deliver light from the distal tip to cure patch material at some position distal to the tip of the catheter, then after that retraction of that needle, deliver light from a more proximal position within the catheter to break some of the chemical and/or physical bonds of the patch material to prevent disfunction of a subsequent biopsy procedure or contamination of the biopsy sample.

### 6. Balloon Aided Patch Placement

In some embodiments, the tissue repair device comprises an inflatable balloon within the patch material compartment. In some embodiments the balloon is inflated to assist with excreting a patch material. In some embodiments, the balloon is inflated to apply pressure to a site for tissue repair (e.g., a biopsy site). In some embodiments, the balloon is inflated to apply pressure to a patch material as it is applied to a site for tissue repair.

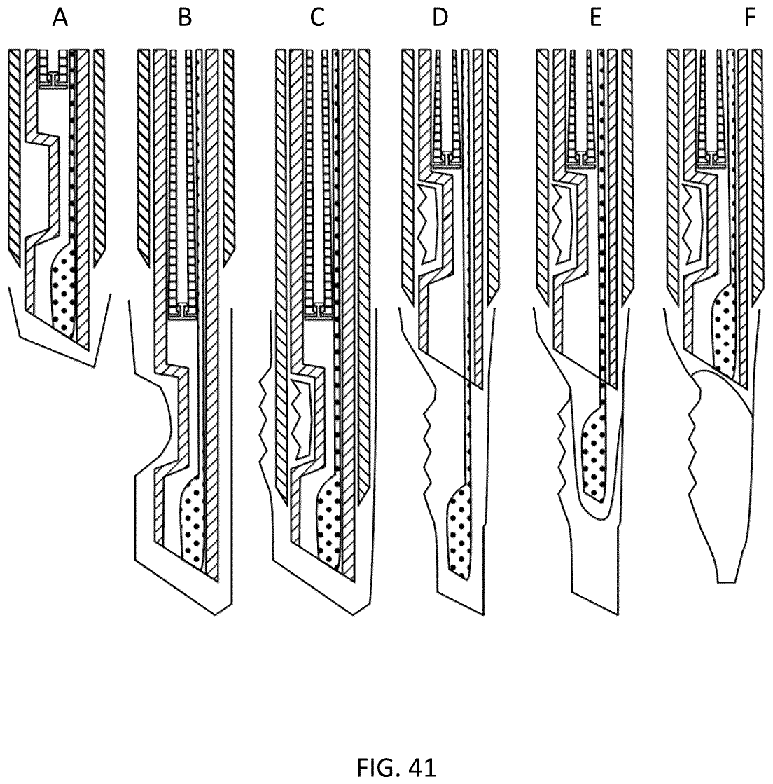

In some embodiments, as depicted in FIGS. 41A-41F, a balloon may inflate to expel patch material from the device. In some embodiments, the balloon applies pressure to the patch material as the patch material is being applied to a site for tissue repair. In some embodiments, after application of an initial volume of patch material, the balloon retracts into the device. In some embodiments, after retraction, additional patch material is applied into the space vacated by the balloon.

In some embodiments, the balloon is filled using a fluid. The device may comprise a balloon fluid reservoir to contain fluid used to fill the balloon. In some embodiments, the balloon fluid reservoir is pressurized such that the fluid fills the balloon upon actuation. In some embodiments, the device comprises a valve or actuator which, upon activation, provides the balloon in fluid communication with the balloon fluid reservoir. In some embodiments, a vent is in fluid communication with the reservoir. In some embodiments, venting of the reservoir allows for decreasing pressure such that the balloon may retract back into the device due to tension forces of an elastomeric material of which the balloon is comprised from.

In some embodiments, the balloon is comprised of polyurethane, silicone, another suitable elastomeric material, or a combination thereof. In some embodiments, the balloon comprises a coating. In some embodiments, the coating is hydrophobic. In some embodiments, the coating is hydrophilic. In some embodiments, the coating is selected to reduce adhesion to one or more patch materials utilized in the device.

### E. Optic Sensor

In some embodiments, the device comprises one or more optic sensors comprising: photo-diode surfaces; nanopatterned FO-SPR sensor tips; touch imprint cytology (TIC); charge-coupled devices (CCDs) (may be silica or IR coupled devices); analog (voltage or current) and digital (pulse-counting) domains; a single detector; a multidetector; a photomultiplier tube PMT (for spectroscopy and or microscopy); a time gated PMT; linear CCDs (and photodiode arrays); one or more CCDs; “Open Electrode” (or “Open Poly”) CCD; Silicon CCDs; basic, front-illuminated CCDs (e.g. coated with a phosphor to enhance its UV response); Electron Multiplying CCD (EMCCD); Intensified CCD (ICCD) detectors; or combinations thereof.

In some embodiments, optical sensors sense the environment and/or surrounding biological tissue types. In some embodiments, data received by the sensors is used to automate one or more processes performed by the device or provide feedback to a user of the device.

### F. Patch Material Sensors

In some embodiments, the device comprises patch material sensors. In some embodiments the patch material sensors comprise one, two, or more sensors including bioimpedance sensors, optical sensors, pressure sensors, other sensors, other sensors, or a combination thereof.

In some embodiments, the patch material sensor is used to detect the degree of polymerization while still within a portion of the instrument. This can be important to ensure the correct amount of polymerization has taken place before injecting the patch material into the target space, so that the patch material can have a tensile strength and viscosity to resist the pressures and turbulent forces related to bleeding at arterial pressures. Alternatively, these sensors can be used to ensure the patch material components or mixture have the correct composition for the intended organ or purpose. For example, in some embodiments, one two or more NIR spectrum short bands could be quickly analyzed by the sensor, which can be used to determine water molecule concentration relative to solutes. Other molecules can be detected in NIR and other bands of absorbance spectroscopy testing.

### 1. Patch Material Information Display

In some embodiments, the device comprises information to the user to provide information about the patch material level, readiness for use, number of available doses, priming status, type, intended organ, seal strength, information about mixing components, readiness for mixing component, completion of mixing, viscosity information, volume intended to inject, warning for low level, other warning, expiration date, curing status, empty status, mechanism status, other information about the patch material, or some combination thereof. In some embodiments, the device the patch information display provides information about one, two, or more patch materials, substances, cells, components, mixtures, additives, seal strength, or some other components thereof.

In some embodiments, the patch material information displays information on digital display, LED screen, ultrasound guidance viewing screen, CT guidance viewing screen, MRI guidance viewing screen, some other electronic viewing screen, indicator lights, physical window to the patch material, color display, some other physical display, some other display, some other indicator system, or a combination thereof.

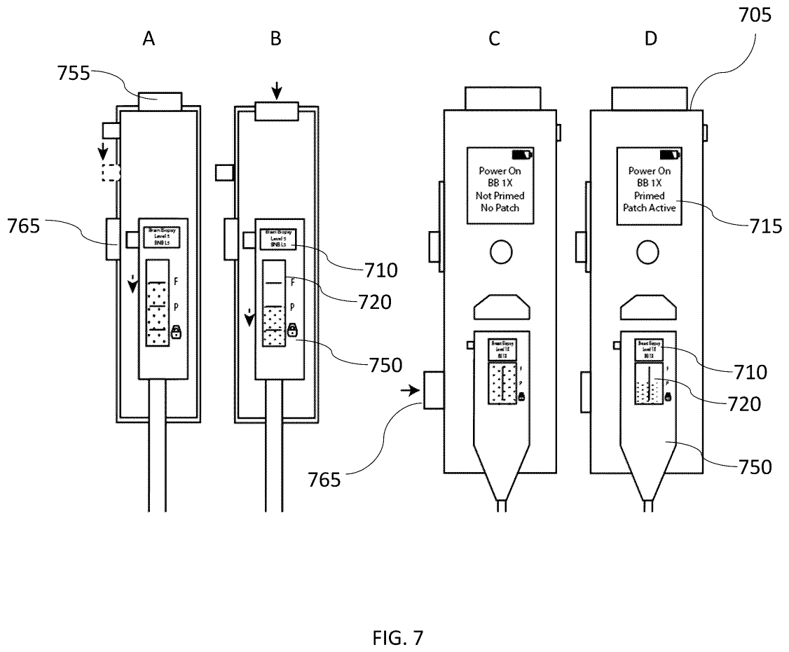

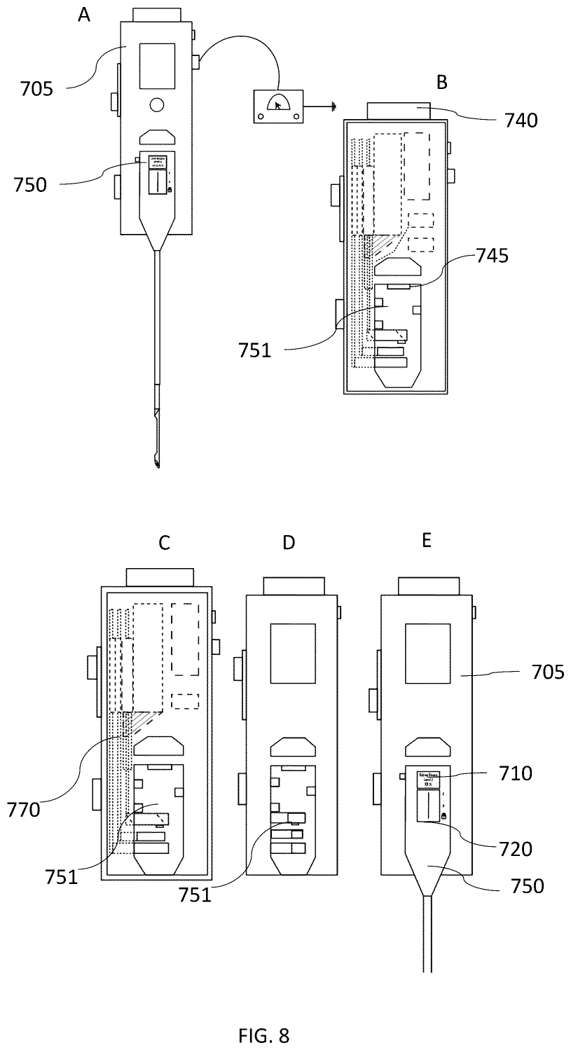

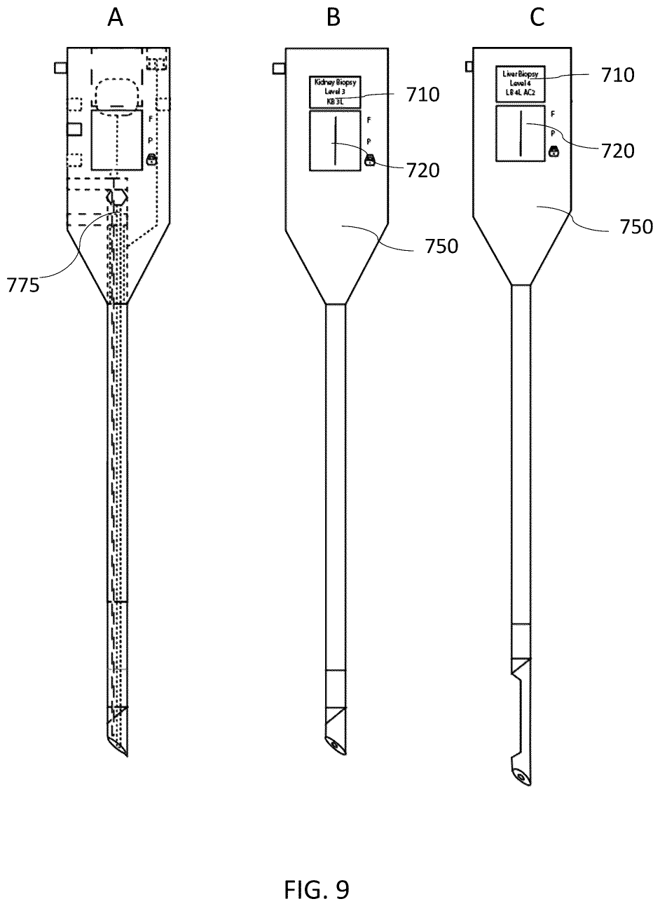

With reference to FIGS. 7A-9C, a patch material information displays are depicted, according to some embodiments. In some embodiments, an information display is provided on a needle cartridge. In some embodiments, a needle cartridge 750 comprises a procedure display 710 providing information as to the type of procedure to be performed. In some embodiments, the patch material is prepared based on the procedure to be performed. In some embodiments, the needle cartridge 750 comprises a gauge 720 to display the amount of patch material remaining in the cartridge.

In some embodiments, a device comprises a controller 705 which receives a needle cartridge. In some embodiments, the controller comprises a display 715 which provides information regarding a status of the controller, a status of a procedure, a status of the cartridge, or other information which may be useful in performing a biopsy and/or repairing a biological wound site. In some embodiments,

In some embodiments, the patch material information display comprises a window to physically see the patch material within one, two, or more portions of the device. In some embodiments, the patch material information display has a window for user to physically view the patch material. In some embodiments, the patch material information display comprises indicators which correspond to different levels of patch material within the device, prime status, ready status, number of available uses, empty status, patch material release information, conveys information as described above or some combination thereof.

In some embodiments, the device comprises one, two, or more patch material information displays which are both physical and electronic in nature. In some embodiments, the device comprises one, two, or more patch material information displays which includes both a physical window to view patch material components and an electronic indicator mechanism. In some embodiments, the device comprises one, two, or more patch material information displays which includes both a physical window to view patch material components and an electronic based indicator mechanism. Having the combination of both types of information may facilitate visual analysis, as some patch materials may polymerize before use, leak, premix, become contaminated, or be otherwise altered or denatured before use which at times may only be detectable through visible analysis by the user. Once this patch material is compelled, pushed, moved, mixed, activated, or otherwise altered, the user may need to rely on other displays and sensors because some information may not be detectable to the user. To safely inject the patch material, especially for uses such as stopping arterial-pressured bleeding, the device may therefor need to have both a window patch material display and a digital display or indicator system.

### G. Instrument Cartridge

In some embodiments, the device is comprised of a housing: removeable instrument cartridge comprised one or more biopsy needle: mechanism for photopolymerizable patch material injection and wound sealing: a single user-controlled mechanism to turn on or off both the patch delivery and curing mechanisms: components and controls for a standard biopsy procedure; and a light source.

In some embodiments, the device is comprised of a housing; removeable instrument cartridge comprised one or more biopsy needle: mechanism for photopolymerizable patch material injection and wound sealing: a single user-controlled mechanism to turn on or off both the patch delivery and curing mechanisms: components and controls for a standard biopsy procedure; and a light source. In some embodiments, the device is comprised of a housing; removable instrument cartridge comprised one or more biopsy needle; one or more photon projection components to partially polymerize patch material components within a lumen of the device before delivery to and further polymerization at a separate target location: mechanism for photopolymerizable patch material injection and wound sealing; one or more user-controlled mechanisms to turn on or off both the patch delivery and curing mechanisms; components and controls for a standard biopsy procedure. In some embodiments, the device is comprised of a housing: removable instrument cartridge comprised pf one or more biopsy needle; one or more photon projection component to partially polymerize patch material components within a lumen of the device before delivery to and further polymerization at a separate target location: mechanism for photopolymerizable patch material injection and wound sealing; components for an ablative step; one or more user-controlled mechanisms to turn on or off the patch delivery and curing mechanisms; components and controls for a standard biopsy procedure.

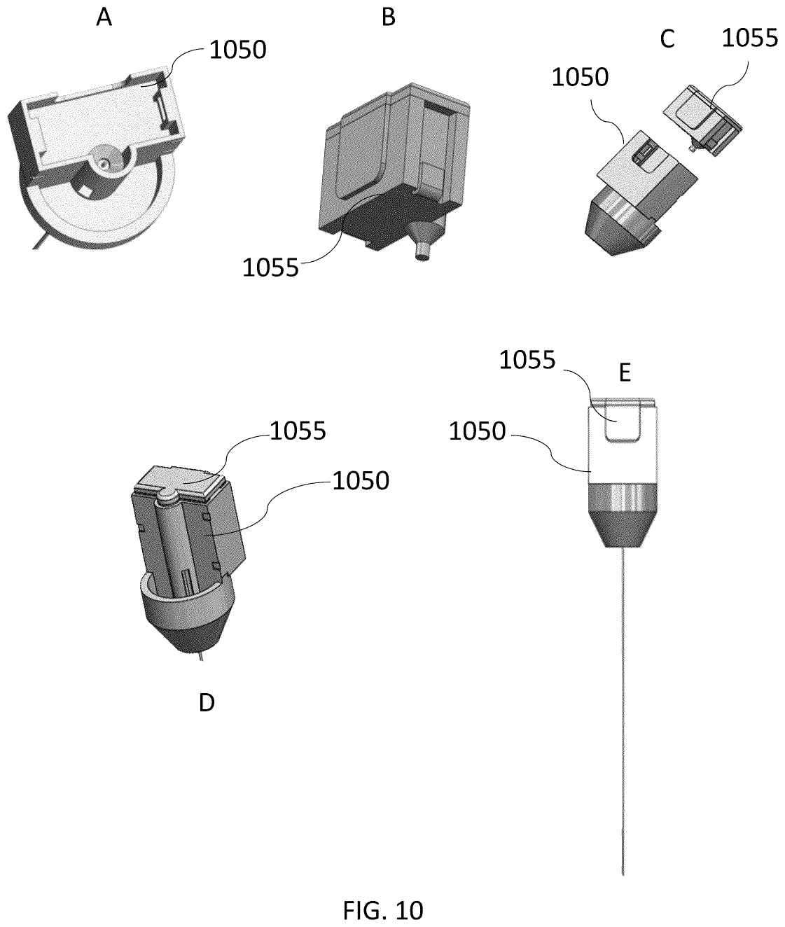

FIGS. 10A-10E depict a needle cartridge 1050 and patch material reservoir 1055. In some embodiments, the reservoir mates within the needle cartridge. In some embodiments, depression of the reservoir in a distal direction causes the patch material to be emitted from a distal end of the needle. In some embodiments, mating of the reservoir with the cartridge facilitates selection of patch material, as disclosed herein.

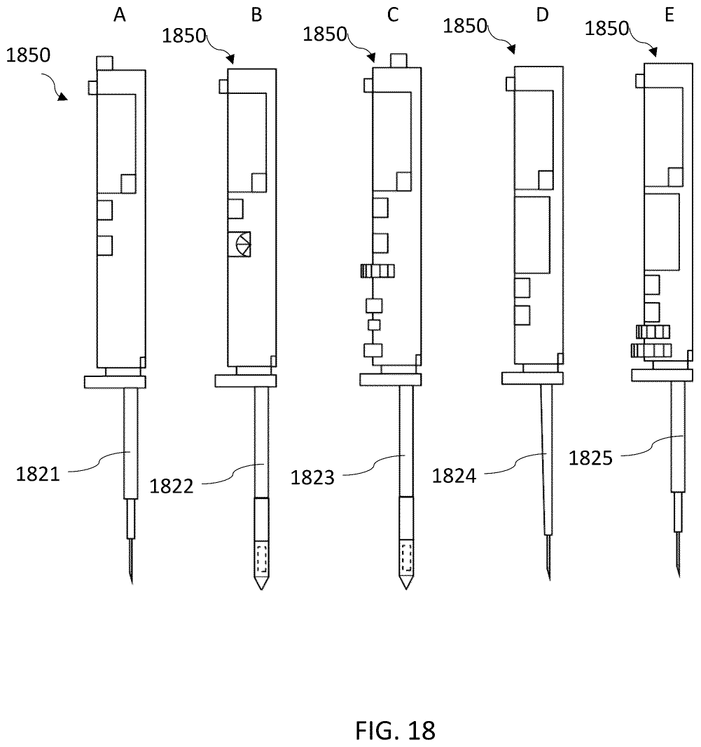

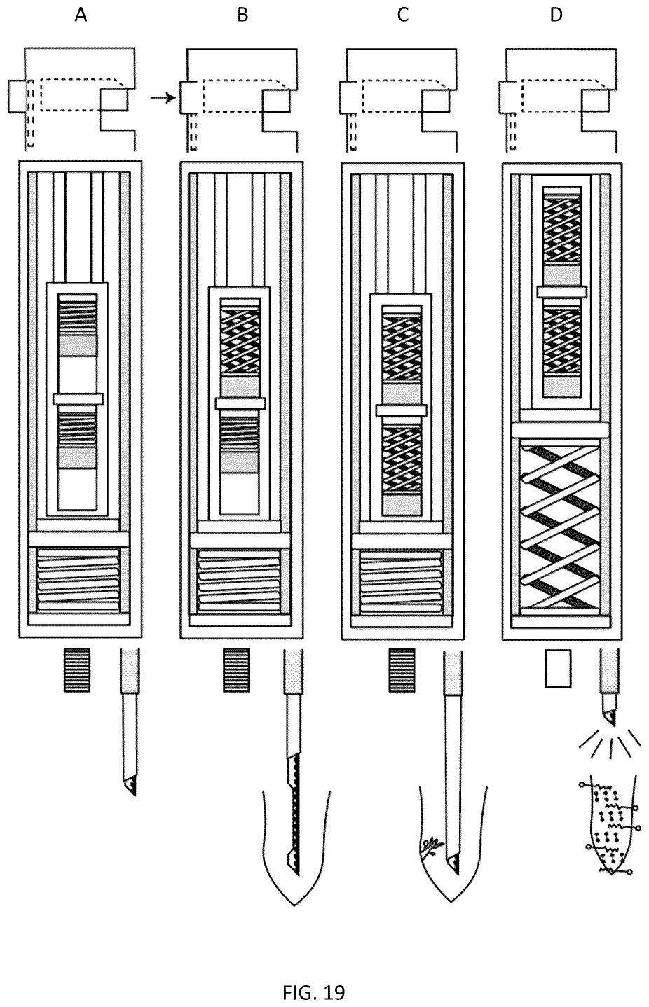

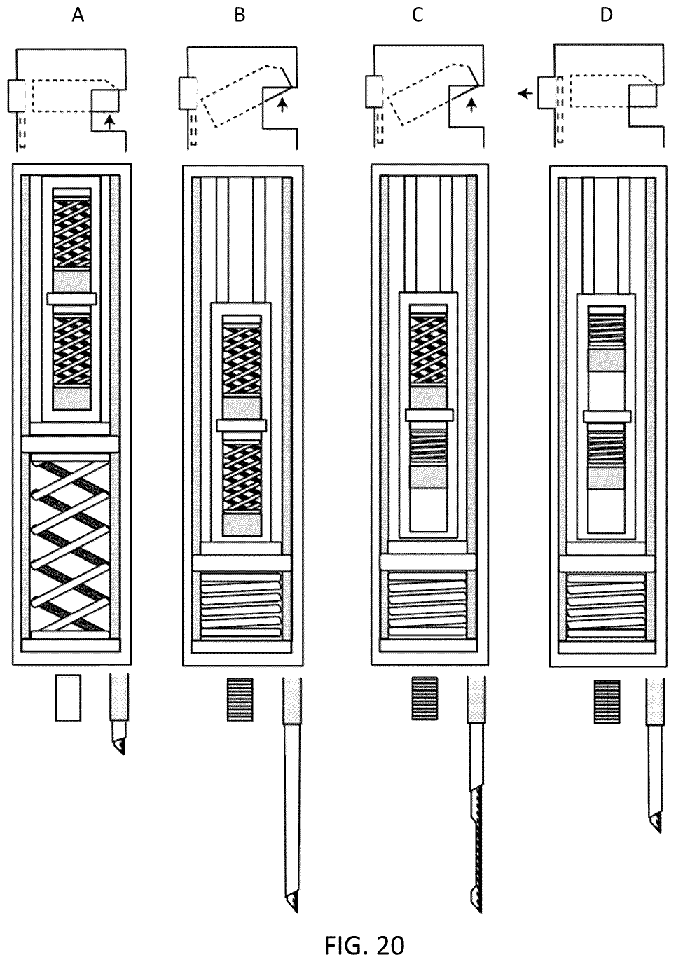

In some embodiments, as depicted in FIGS. 18A-18E, a needle cartridge 1850 may comprise one or more components which interface with a controller when the needle cartridge is placed within the needle cartridge recess of the controller. In some embodiments, the recess provides components for manual/mechanical translations of components of the needle cartridge. Further, upon receiving the needle cartridge, electrical components may be placed in electrical communication with corresponding electrical components of the needle cartridge. In some embodiments, a controller may be adapted for use with multiple needle types, such as retractable telescoping needles 1821, 1823, 1825 and biopsy needles 1822, 1823. Further, controllers may be adapted to acuate different mechanical components provided on a needle cartridge. For example, the needle cartridge of FIG. 18C may interface with a horizontal gear actuation system of a controller, as depicted in FIG. 30B.

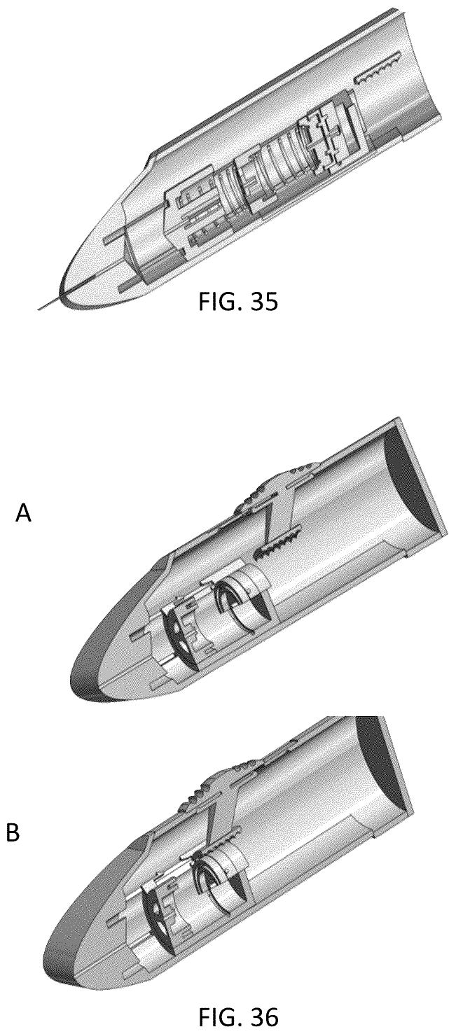

FIG. 35. depicts an automated needle retraction system, according to some embodiments. In some embodiments, the retraction system comprises a spring and teeth which engage with a needle carrier. In some embodiments, when the needle carrier extends beyond the teeth, the spring biases the needle carrier in a proximal direction, retracting the needle.

FIGS. 36A and 36B depict a needle retraction system comprising a slider, engageable by a user to retract a needle after use.

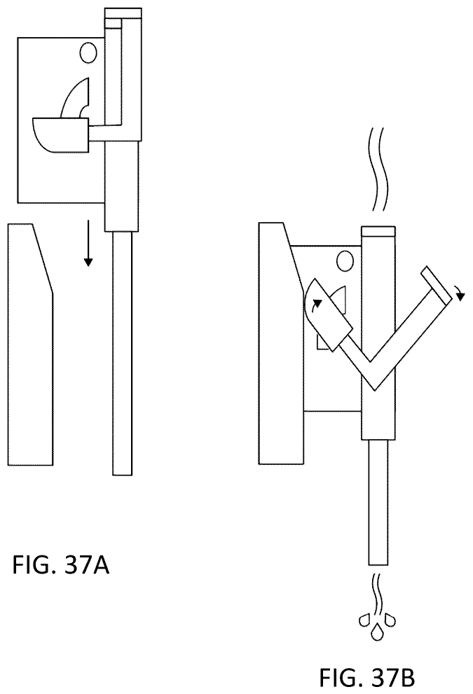

FIGS. 37A-37B depict a system for automated activation of a light source for initiating photopolymerizable materials. In some embodiments, as the needle carrier 3705 is translated downward and emits the photopolymerizable materials, lever 3710 abuts an edge, causing rotation of the lever. In turn the lever may an activation switch to activate the light source. This system may allow timing of a light source activation with dispensing of a photopolymerizable material.

In some embodiments, the device is comprised of a housing: removable instrument cartridge comprised one or more biopsy needle and locking mechanism that prevents repeat biopsy sampling after one application patch material; mechanism for photopolymerizable patch material injection and wound sealing; one or more user-controlled mechanism to turn on or off the patch delivery and curing mechanisms; components and controls for a standard biopsy procedure; and a light source. In some embodiments, the device is comprised of a housing: removable instrument cartridge comprised one or more biopsy needle and locking mechanism that prevents repeat biopsy sampling after one application patch material; one or more photon projection component to partially polymerize patch material components within a lumen of the device before delivery to and further polymerization at a separate target location; mechanism for photopolymerizable patch material injection and wound sealing; one or more user-controlled mechanism to turn on or off the patch delivery and curing mechanisms; components and controls for a standard biopsy procedure; and a light source.

In some embodiments, the device is comprised of a housing: removable instrument cartridge comprised one or more biopsy needle; a locking mechanism that prevents repeat biopsy sampling after a limited number of applications patch material; one or more photon projection component to partially polymerize patch material components within a lumen of the device before delivery to and further polymerization at a separate target location such that the delivery and seal can withstand bleeding pressures up to approximately 250 mmHG with every application; mechanism for photopolymerizable patch material injection and wound sealing; one or more user-controlled mechanism to turn on or off the patch delivery and curing mechanisms; components and controls for a standard biopsy procedure; an ablative step; and a light source.

In some embodiments, the device is comprised of a housing: removable instrument cartridge comprised one or more biopsy needle; a locking mechanism that prevents repeat biopsy sampling after a limited number of applications patch material; one or more photon projection component to partially polymerize patch material components within a lumen of the device before delivery to and further polymerization at a separate target; a patch material cartridge which can be selected by the user according to one or more variables related to the intended use; mechanism for photopolymerizable patch material injection and wound sealing; one or more user-controlled mechanism to turn on or off the patch delivery and curing mechanisms; components and user controls for a standard biopsy procedure; and a light source.

In some embodiments, the device is comprised of a housing; removable instrument cartridge comprised one or more biopsy needle, one or more attachment points for attachment to a separate image guidance system, and one or more attachment points for external robotic actuator controls; a locking mechanism that prevents repeat biopsy sampling after a limited number of applications patch material; a patch material cartridge which can be selected by the user according to one or more variables related to the intended use; mechanism for photopolymerizable patch material injection and wound sealing.

In some embodiments, the device is comprised of a housing; removable instrument cartridge comprised one or more biopsy needle, one or more attachment points for attachment to a separate imaging based guidance system, and one or more attachment points for external robotic actuator controls; a locking mechanism that prevents repeat biopsy sampling after a limited number of applications patch material; one or more photon projection component to partially polymerize patch material components within a lumen of the device before delivery to and further polymerization at a separate target; a patch material cartridge which can be selected by the user according to one or more variables related to the intended use; mechanism for photopolymerizable patch material injection and wound sealing.

In some embodiments, the instrument cartridge may comprise one or more patch sensors. These patch sensors may be comprised of bioimpedance sensor, pressure sensor, optical sensor, IR sensor, NIR sensor, UV sensor, vibrational sensor, position sensor, viscosity sensor, torque sensor, bending sensor, rotational sensor, other sensor, or some combination thereof. This sensor can be used to determine many unanticipated aspects of the device and patch material, such as monitoring components concentration, viscosity, strength, or other aspects of partially activated patch material within a lumen of the device before injecting into the instrumentation tract to help ensure seal and hemostasis in the setting arterial pressure bleeding.

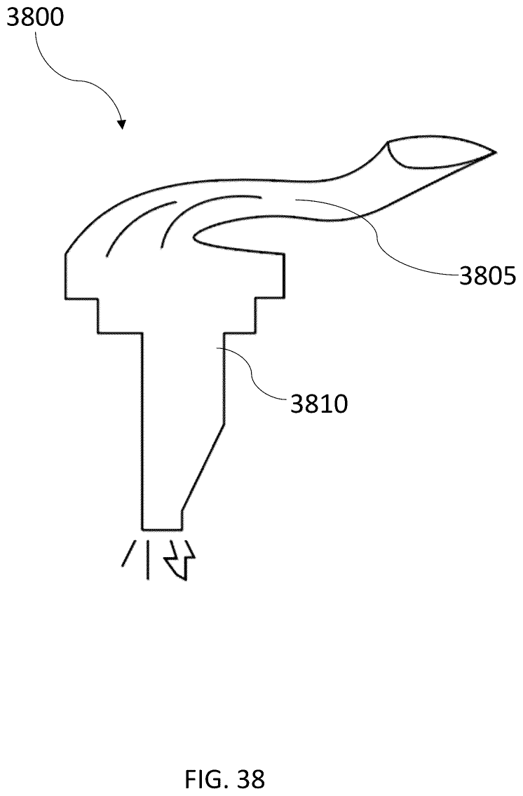

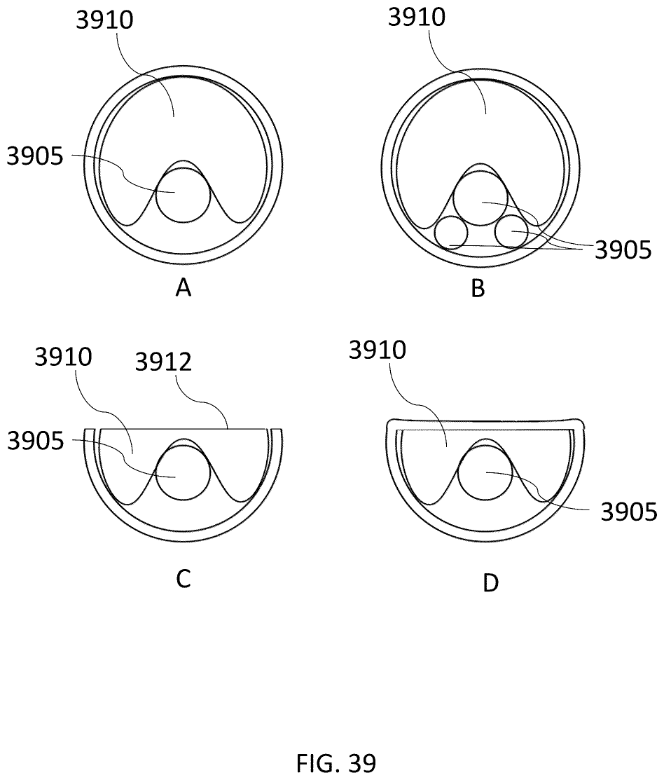

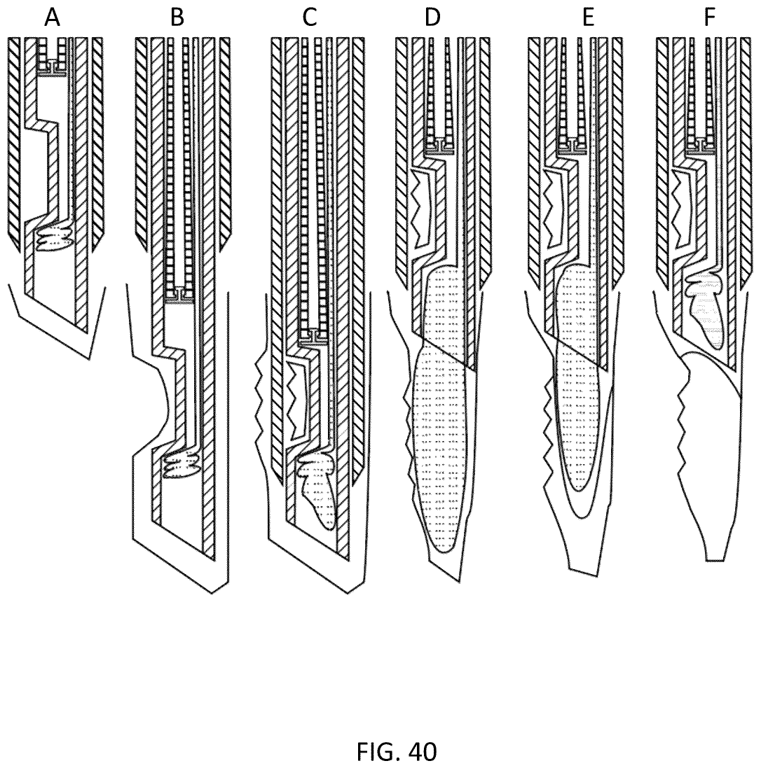

In some embodiments, then instrument cartridge is reusable. In some embodiments, the instrument cartridge contains patch material components in separate chambers before priming. In some embodiments, the instrument cartridge comprises one or more patch material mixing chambers. In some embodiments the instrument cartridge comprises a sterile cover. In some embodiments, the sterile cover comprises an external sterile sleeve, an internal sterile sleeve, a sterile component capsule system, a sterile sleeve trimming system, some other sterile cover, or a combination thereof. An exemplary sterile sleeve 3800 is depicted in FIG. 38, according to some embodiments.

In some embodiments, the instrument cartridge comprises a connector to an external axial drive component which also has a waveguide connection. This can be permanent or reversibly connected. In some embodiments, the instrument cartridge connects to a telescoping waveguide within a separate portion of the device. In some embodiments, the instrument cartridge comprises an integrated low mass light source. In some embodiments, the sterile sleeve permits the passage of photons from a light source for photoinitiation. In some embodiments, the sterile sleeve permits the passage necessary for RF cautery. In some embodiments, the sterile sleeve comprises a ridged base 3805 and a flexible top portion 3810.

In some embodiments, the instrument cartridge comprises a controller. In some embodiments, the instrument cartridge comprises one or more of an external catheter, internal catheter, wave guide within a catheter wall, light diffuser within a catheter wall, light diffuser which allows light to activate light within the lumen of the catheter, extendable catheter, stationary catheter, retractable catheter, or some combination thereof. In some embodiments, the instrument cartridge comprises a plate which ultrasound waves can be transmitted, as depicted in FIGS. 48A and 48B. The plate may comprise a connector 4805 for attaching to a device housing. The plate may comprise an aperture 4810 for allowing a guiding a needle or device.

In some embodiments, as depicted in FIGS. 7A-9C, a needle cartridge 750 may comprise one or more components which interface with the controller 705 when the needle cartridge is placed within the needle cartridge recess 751 of the controller. In some embodiments, the recess 751 provides components for manual/mechanical translations of components of the needle cartridge. For example, depression of button 740 may correspond to translation of actuator 745 to engage the actuation button 755 of the needle cartridge. Further, upon receiving the needle cartridge, electrical components 770 may be placed in electrical communication with corresponding electrical components 775 of the needle cartridge.

In some embodiments, the instrument cartridge comprises a port. This port can serve as a connection to attach one or more syringe, vacuum tube, luer lock, or a combination thereof. In some embodiments, the instrument cartridge can comprise a computer system. In some embodiments the instrument cartridge has one or more patch material sensors located within that portion of the device. Similarly, primer button 760 provided on the controller may engage primer button 765 provided on the needle cartridge.

In some embodiments, the instrument cartridge can comprise a vacuum system to assist with biopsy. In some embodiments, the instrument cartridge comprises one or more suction attachment areas to attach the cartridge to a biological site. Suction during photopolymerized patch sealing of these biopsy wounds can also improve the fluidity of motion of the device, so the patch material column is continuous and not interrupted by patient movement. This also allow optimal axial movement rate of the device components specific to a patient's needs, organ, disease etiology, and disease severity. during patch column creation within the wound tract. This also allows better assessment of bleeding before use of patch material which may allow for additional biopsies before changing the cartridge. For example, during prostate biopsy, the suction areas can be comprised of a curved surface with diameter small enough to be easily introduced into the rectum, while also stabilizing the device to anal tissue. Suction from the curved surface during rectal biopsy also increases force on the tissues between the biopsy site and to that of the can better maintain tissue alignment during can keep the ultrasound probe and needle aligned during a biopsy procedure, and improve photopolymerized patch material sealing and propagation by better maintaining alignment of the needle with the probe head.

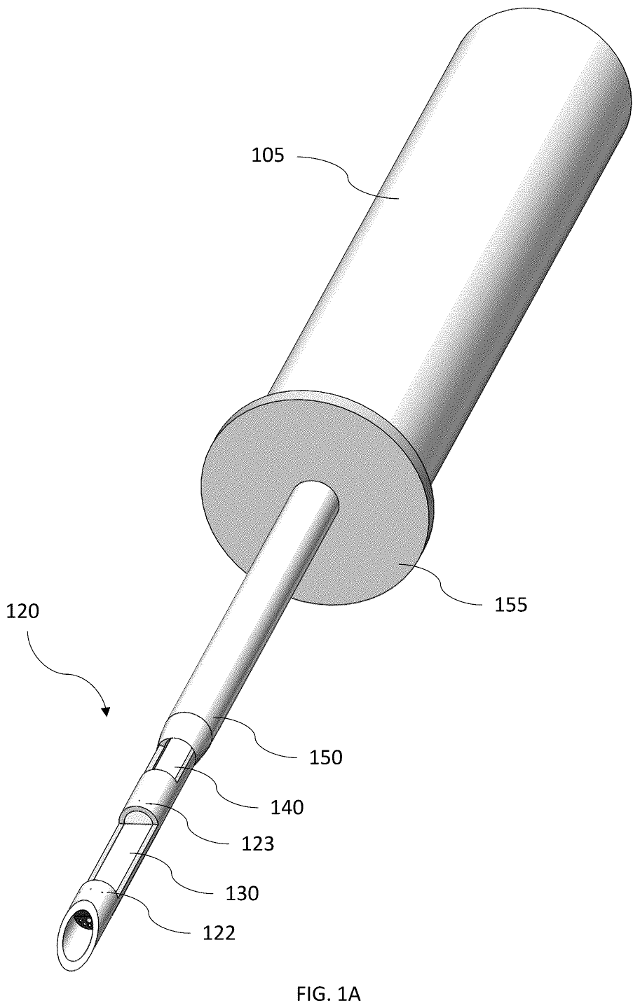

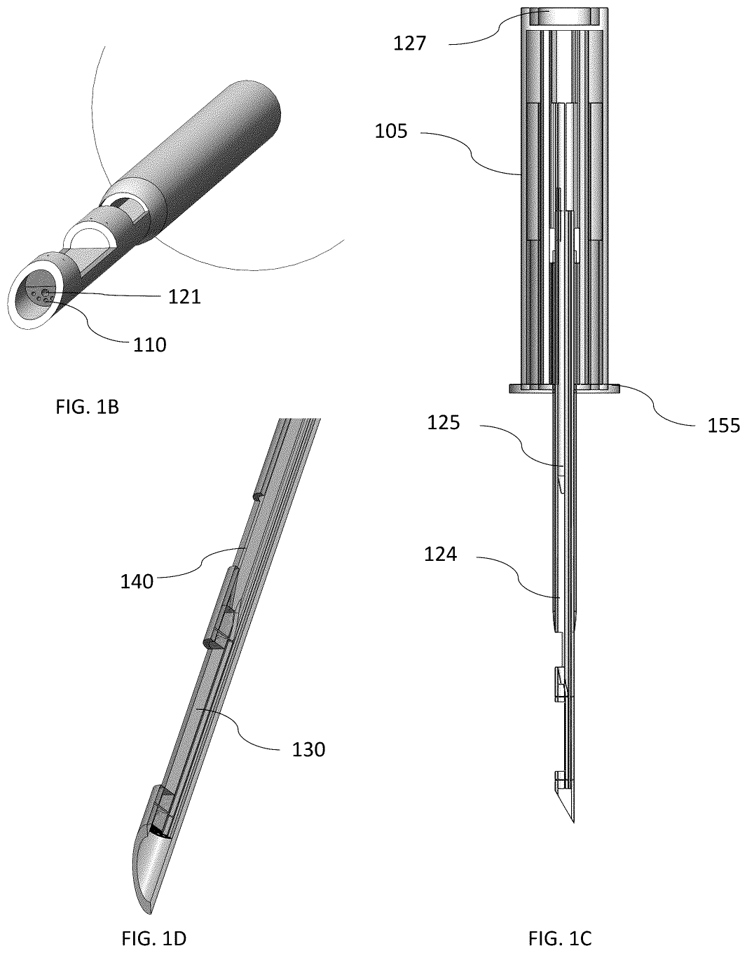

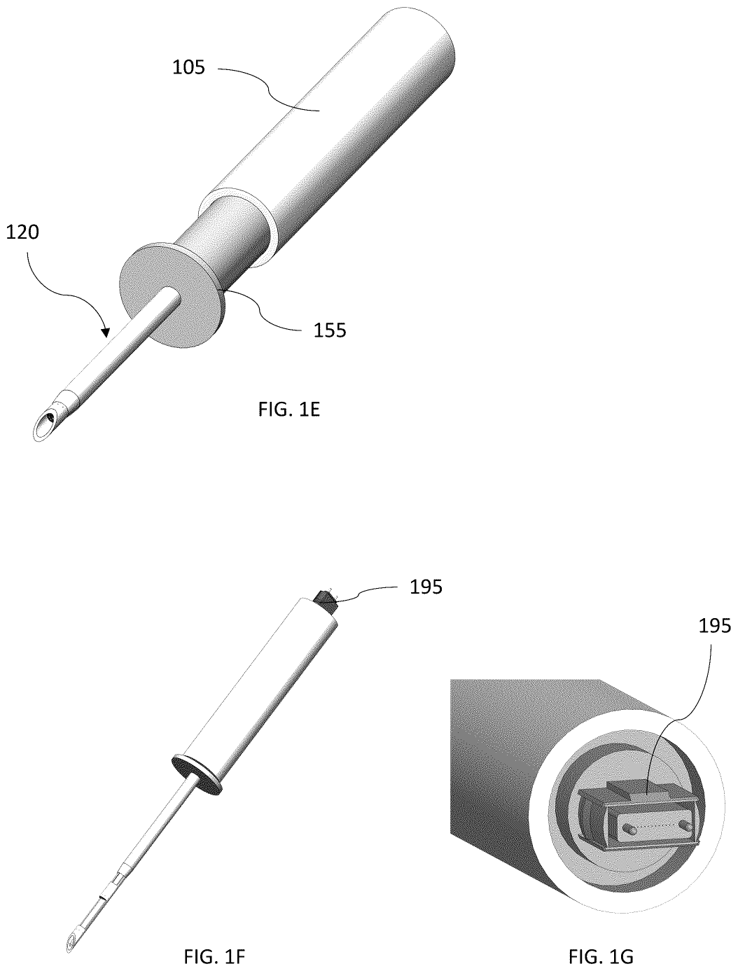

In some embodiments, the instrument cartridge comprises external attachments for an RF cautery energy source. In some embodiments, the instrument cartridge comprises external attachments for an RF cautery energy source and a light source for activation of photopolymerized gel (e.g., see 195 in FIGS. 1F and 1G). In some embodiments, the instrument cartridge comprises external attachments for a monopolar RF cautery energy source and a light source for activation of photopolymerized gel. In some embodiments, the instrument cartridge comprises external attachments for a bipolar RF cautery energy source and a light source for activation of photopolymerized gel. In some embodiments, the instrument cartridge comprises external attachments for a monopolar RF cautery external pad and a light source for activation of photopolymerized gel.

### H. Light Source Controller

In some embodiments, timing, wavelength, wavelength variability of, intensity, intensity variability, amount of, location emitted from, location transmitted to, portion of the device involved with, generation of, portion of device generated from, intensity of all, intensity of a portion of, shape, aim, target of, type of transmission, duration of, pause between, target of all, target of a portion of and/or other aspects or some combination thereof the photons released from the device relaying are timed with automatic timing by the device; with manual timing by the device; with semi-automatic timing by the device; with timing related to the photoactivated substance ejection; with timing for immediate release of photons; with timing for delayed release of photons; with timing before, during, or after a procedure is completed, or some combination thereof; with timing before, during, or after a needle is inserted, or some combination thereof; with timing before, during, or after a tubular structure is entered or some combination thereof; with any combinations thereof, or sometimes the photons are not released at all.

In some embodiments, the extent to which a substance has undergone-photochemical activation can be detected using optical input examining the target region, and the device can notify the user of that progress, and/or make computer based and/or computer aided decisions as to when, and/or how long photons will be emitted from the device. In some embodiments, light source emission is controlled relative timing related to the shape, structure, stricture, internal flow, internal pressures, luminal wall pressures, hemorrhage, oncotic pressures, physiology, pathophysiology, damage to, missing, removed, excised, manipulated, added, repaired, surgical portion and/or other features of an organ sensed, detected, calculated, and/or proposed by the user and/or computer software.

In some embodiments, light source emission is controlled to prevent photoactivated substance adherence to the device; on (and/or related) insertion of the needle; on (and/or related to) retraction of the needle; or combinations thereof. In some embodiments, the light source comprises a variable wavelength to change intensity, depth of penetration, or affect. In some embodiments, light is emitted from a variable location to target or avoid certain biological substances, tissues, cells, liquids, or materials, to activate certain locations or positions while not activating others, to create structures, or a combination thereof.

### I. Ancillary Sensors

In some embodiments, the device comprises one or more ancillary sensors to retrieve data related to a bioimpedance of substance; a bioimpedance of organ; a bioimpedance at biopsy cut location; a bioimpedance of a needle; a bioimpedance of a vessel; a bioimpedance of a wound; a bioimpedance measure of a biocompatible patch; a bioimpedance measure of needle portion in target organ; a bioimpedance of portions of the substrate; and combinations thereof.

In some embodiments, ancillary sensors utilize diffuse reflectance spectroscopy and/or infrared (IR) thermography to analyze the surrounding environment/tissue. In some embodiments, sensors monitor the deposition and/or curing of the biocompatible patch material in real time. In some embodiments, the state of the biocompatible patch material is monitored by Fourier-transform infrared (FTIR) spectroscopy, X-ray diffraction analysis, bioimpedance testing, visual spectrum spectroscopy, other spectroscopy testing, and combinations thereof.

In some embodiments, ancillary sensors allow obtain positional data of the device to locate, label, and store information such as the location of a biopsy site and the number of biopsy locations for later reference. In some embodiments, positioning/inertial sensors include accelerometers, gyroscopes, and combinations thereof.

In some embodiments, hybrid nanostructured films containing graphene nanoplatelets (GNPs) and/or double-stranded DNA are utilized for in situ and real-time detection of UV radiation damaging effects from the changes of the film electrical properties induced by exposure to UV-C radiation.

In some embodiments ancillary sensors are utilized to perform one or more of the following actions: confirming placement of the biocompatible patch material; confirming proper location of a biopsy site; detecting the angle, orientation, and/or the depth of a needle instrument within a tissue; detecting what portion of the needle is within what tissue, what aspect of tissue, or related structures; analyzing tissue for disease and/or diagnosing disease; analyzing tissue for presence and/or type of second disease, third disease, etc.; examining types of cells and their status of health and or disease; and examining the extracellular materials.

### J. Thermal Source

In some embodiments, the device comprises a thermal source. In some embodiment, the thermal source comprises a thermoelectric or Peltier device for providing heating and/or cooling effects. In some embodiments, the thermal source is used to cauterize tissue. In some embodiments, the thermal source is used to increase of adherence to the device or other portion of the sealant. In some embodiments, the thermal source is used decreased adherence to the device or other portion of the sealant. In some embodiments, the thermal source is used increase or decrease the amount or rate of substance released from the sealant, stent, or device. In some embodiments, a thermal source regulates temperature for photo sensitive gels which require different temperatures to maintain form.

In some embodiments, there is an electrosurgical control unit. The electrosurgical control unit may control circuitry aspects of the device. In some embodiments, the electrosurgical control unit receives information from one or more sensors, as described herein. In some embodiments, the electrosurgical control unit activates and deactivates components of the device. In some embodiments, the electrosurgical control unit regulates a power supplied to the light source, thermal source, cauterizing component, and other electrical components of the device described herein.

### III. Form Types

Components of the tissue repair device, as disclosed herein, may be incorporated into various medical/surgical tools or instruments to allow for such instruments to be utilized as a tissue repair device while performing their intended function as well. In some embodiments, the components of the tissue repair device may be incorporated into: vacuum biopsy needles, multiple vacuum biopsy devices, core biopsy needles, fine needle biopsy devices, high velocity needles, rotating biopsy needles, aspiration biopsy devices, cyrobiopsy devices, pneumatic pulse activated biopsy devices, injection needles, one-handed biopsy devices, two-handed biopsy devices, modular biopsy systems, disposable biopsy systems, and multiple cocking biopsy devices.

### A. Light Rod

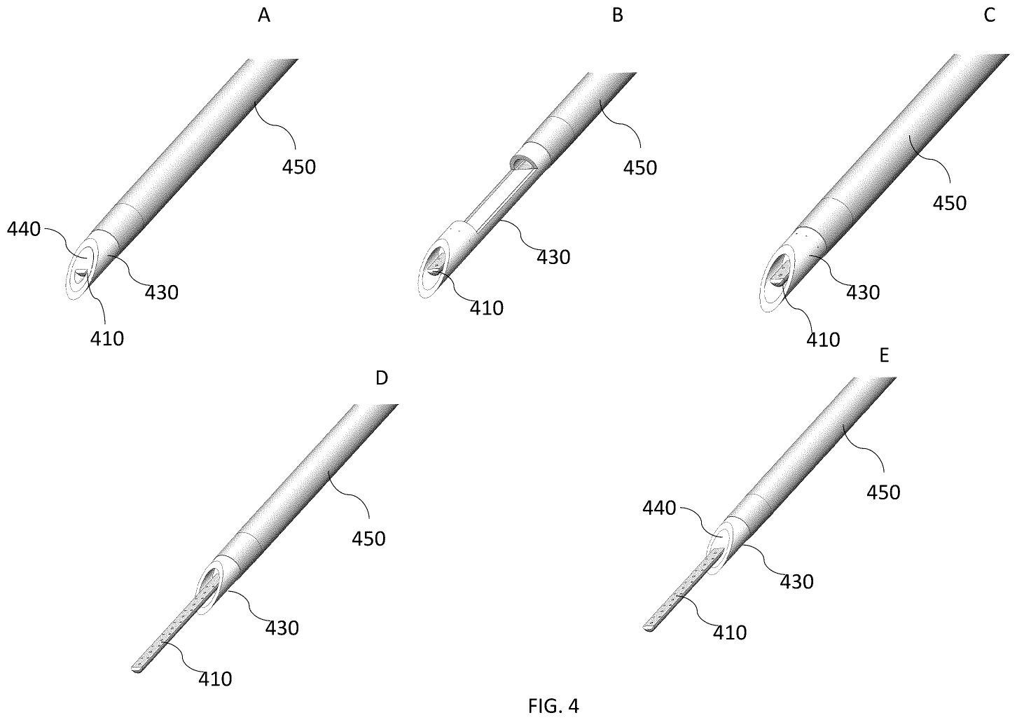

In some embodiments, as depicted in FIGS. 4A-4D, there is a portion of the device that can be extended beyond the distal end of the needle. In some embodiments, this is an unanticipated advantage because a larger portion of the biopsy bed can be covered and sealed with patch material, as compared with photons that project only from the distal end of the needle or some portion within the side of the needle. The location of the projected light can also give the advantage of having a more uniform seal created in the biopsy tract and/or the biopsied tissue bed. This portion of the device can also give an unexpected advantage of decreasing cost and/or difficulty of manufacturing. The other advantage to this, is that a scaffold or sheath can be advanced and the same time. This scaffold can be used to seal the seal the tissue, stabilize the patch material, or put radial and/or distal external pressure on the surrounding tissues.

In some embodiments, a first step is depicted in FIG. 4A. In some embodiments, all components are in their initial positions. The components include an outer sheath 450, biopsy core needle 430, middle rod sheath 440, and inner light rod 410. The core needle, middle rod sheath, and inner light rod are aligned at their distal tip, and the outer sheath is fully covering the biopsy trough of the biopsy core needle.

In some embodiments, a second step is depicted in FIG. 4B. In some embodiments, once the initial assembly is close to and aligned with the desired biopsy target, the user activated the biopsy action. This action compels the biopsy core needle, middle rod sheath, and inner light rod forward, and the outer cutting sheath remains in the same location. Once fully extended, the middle rod sheath is then pulled back within the lumen of the biopsy core needle. As it retracts, photon activated pated material is ejected into the new space within the center lumen of the biopsy core needle. The inner light rod may or may not begin to activate the patch material that surrounds it within the center lumen of the biopsy core needle.

In some embodiments, a third step is depicted in FIG. 4C. In some embodiments, the outer cutting sheath is advanced distally in axial alignment with the inner core needle, which cuts and encapsulates the target tissue within the biopsy trough of the biopsy core needle.

In some embodiments, a fourth step is depicted in FIG. 4D. In some embodiments, the outer cutting sheath, biopsy core needle, and middle rod sheath are the retracted some distance within the tissue, maintaining the same location relationship to one another. The Inner light rod is relatively advanced at the same time such there little to no linear movement of the inner light rod relative to its location within the tissue. This fully exposes the light rod to the tissue bed from which the biopsy was cut, while not further penetrating that tissue. This also prevents unintended penetration of the photo delivering component of the device into tissue distal to that already entered by the core needle itself, and/or any other portions of the needle. In some embodiments the inner light rod can both project photons and deliver patch material. The portion of the gel which has already been partially activated may be released distal to the biopsied tissue in the axial direction, located at or around the same location, or proximal to the location. The partially activated material may serve an anchor for more patch material, it may serve as a scaffold to stabilize the location of the patch material, it may not be present at all. More gel is compelled into the needle tract and the light is activated. In some embodiments, there is shape of the inner light rod has no sharp edges. In some embodiments the edges and or the tip are rounded, oval, semicircular, or shaped in such matter to decrease the likelihood of injuring to distal or surrounding tissue. In some embodiments there is more radial distance between the inner light rod and the biopsied tissue bed than the tissue in one or more of the other radial directions. This is an advantage because more patch material can be placed between the light rod and biopsied tissue bed. In some embodiments there is less radial distance between the light rod and the biopsied tissue bed as compared to one or more of the other directions. This is advantage under some circumstances because the inner light rod is closer to the biopsied tissue bed.

In some embodiments, a fifth step is depicted in FIG. 4E. In some embodiments, the middle rod sheath is then advanced distally within the center lumen of the biopsy core needle. In some embodiments, this pushes the patch material free of the biopsy core needle.

In some embodiments, the Inner light rod is then retracted into the middle core needle. In some embodiments, this action pushes the patch material free of the inner light rod. In some embodiments the inner lumen of the biopsy needle and/or middle rod sheet has an internal radial shape similar to that of the exterior radial shape of the inner light rod. In some embodiments this is an advantage because it can more effectively free the patch material from the external surface of the inner light rod.

In some embodiments, the area around all or a portion of inner light rod can be filled with patch material while the light rod is located within the center lumen of the core biopsy needle. In some embodiments all or a portion of this material can be partially or fully activated before being expelled from the distal tip of the needle. In some embodiments this can serve as a plug such that it more capable of preventing hemorrhage or having portions of activated or non-activated material be carried away from the site of the needle biopsy tract and/or biopsied tissue bed. This provides the unanticipated benefit of preventing patch material from embolizing, and/or prevent the patch material from disrupting tissues, cells, or physiological functions away from the target. This would be helpful to prevent portions of the patch material from damaging the tubules of the kidney or being lodged some other portion of the GU tract. This could perform a similar function in the liver, preventing patch material from embolizing and/or move within organ into a location in the bile tract where it might cause cholecystitis or bile tract blockages.

In some embodiments, the inner light bar does not all activate at the same time, such that only portions of its project photons in a controlled manner. In some embodiments only portions of the inner light rod activate within the lumen of the inner biopsy needle. This could prevent photons from damaging cellular structures and/or DNA.

### IV. Cauterization of the Biopsy Site

In some embodiments, portions of the device are configured to comprise tissue altering components such as a cauterizing other tissue altering mechanisms. Cauterization or tissue alteration performed before, during, or after the biopsy (or other instrumentation) may comprise electrical cautery, thermal cautery (including hot and cold cautery), ultrasonic vibrational cautery, chemical cautery, other types of tissue altering mechanisms, or combinations thereof.

### A. RF Electrosurgical Tissue Alteration

In some embodiments, wherein the device utilizes the radiofrequency electrosurgery, when raising the temperature of the targeted tissue to be altered by RF electrosurgery, a target temperature of the tissue is about 60° C. to about 100° C. In some embodiments, the apparatus facilitates hemostasis of high pressure bleeding and blood vessels in the hypertensive ranges (above 140, 150, 160, 170, 180, 190, 200, 210, 220, 230, 240, 250, 260, 270, 280, 290, 300 mm HG). In some embodiments, these effects can cause hemostasis in the presence of diseased tissues, with abnormal innate or exogenous coagulation factors or function, with abnormal platelet function or amount or concentration, damaged or altered blood vessels, altered or diseased tissue that is less compressible, and/or other the presence or absence of other factors increasing the likelihood of significant hemorrhage. This method may be applied during hemostasis by itself, hemostasis during biopsy procedures, in combination with patch material placement, in combination with photopolymerized patch material, and/or in combination other hemostatic treatment, substance, or material. In this range of temperatures, the mechanical and chemical bonds within molecules are broken and/or altered, and homogenous coagulum is formed. Treated at these temperatures, the number, density, mechanical bonds, and/or chemicals of water molecules present are greatly reduced from this tissue, cells, extracellular matrix, molecules, and/or substances. This may facilitate bonding of the biocompatible patch material.

In some embodiments, the RF electrosurgery, ultrasonic cautery, resistive cautery, chemical cautery, or other mechanism can be used without patch material to reduce the risk of bleeding associated with biopsy or other instrumentation. In some embodiments, the user may prefer to only alter or cauterize the bed of tissue on the lateral aspect of the tract from which the biopsy is cut. In some embodiments, this can be accomplished by using very small bipolar cautery surfaces with an adjacent blade to optimize the tissue changes to prevent hemostasis while minimizing overheating. This can be accomplished using rapid measurements and precise control. These measurements can be detected between the cutting surfaces, and may be used for all or a portion of the biopsy cutting process. This technique may involve internally rotating components within the device body, needle, other components, or a combination there of.

The desiccation and dehydration effects along with molecular and substance changes to the RF electrosurgically altered tissue, cells, substances, and molecules heated to these temperatures, create a stronger adhesion site for the patch material of various types, especially adhesion to photopolymerized gels. Treating at these temperatures followed by the use of patch material in contact with these tissues, molecules, and substances, hemostasis can also be greatly enhanced. This is caused by the combination of the increased mechanical and molecular bond types and density is better able to resist the force. In some embodiments, the use of RF electrosurgery to alter tissues in these temperature ranges, along with sealing that tissue to patch material, allows the tissue and/or patch material to improve its self-healing properties. Improved adhesion is facilitated by improved tissue molecular surfaces, decreased water concentration, or some other mechanism.

Because the cells can take up to 6 seconds to be altered by RF electrosurgery at 60° C., in some embodiments, the temperature of the target cells and/or tissue would be 70° C., 75° C., 80° C., 85° C., 90° C., or 95° C., so the desiccation, molecular changes, and/or denaturization actions can occur at increased rate without causing evaporation and gas expansion.

In some embodiments, the biopsy or other surgical device can be physically or wirelessly connected to an electrosurgery control unit, also known as Bowie unit or electrosurgical (ESU). In some embodiments the unit is able to detect, control, change, activate, and deactivate the RF electrosurgical actions of the device. In some embodiments, the ESU has components and signal which actively detect, measure, receive, compute, send, adjust, or otherwise use bioimpedance measurements of the biological tissue planned, undergoing, or completed changes to the target tissue. In some embodiments, the ESU uses impedance detection to alter the intensity, location, wavelength, frequency, activation waveform, measurements of other portions or actions of the device. In some embodiments, the ESU uses this input, data, and computations to optimize and/or control the output of the device, such that it is able to control the type, location, extent, or severity of the tissue changes, molecular changes, types or concentration of desired molecular changes, water content, temperature, desiccation, cautery, or other effects.

In some embodiments, the use of RF electrosurgery increases the amount, number, density, shape, mechanical bonds, and/or chemical bonds of molecules or substances in the target tissue in a way which increases their adhesive forces with the patch material. In some embodiments, these include increased number or density of hydrophobic amino acids available for bonding to patch materials. These may include one or more of the following amino acids: alanine (Ala), valine (Val), glycine (Gly), proline (Pro), phenylalanine (Phe), methionine (Met), and tryptophan (Trp), isoleucine (Ile), and leucine (Leu). In some embodiments, the treatment of the tissue, cells, and/or extracellular matrix by electrosurgical RF increases the strength of mechanical bonds and/or chemical bonds between those structures and the patch material. In some embodiments of the device, the RF treated tissue, cells, molecules

Above 200 F may cause carbonization, and would result in increased tissue damage. In some embodiments, bipolar electro cautery facilitates sensing of the temperature and impendence of the specific tissue that is altered by the RF electrosurgery. This is important to use along with photopolymerized patch material (or other type of sealing patch material) because you can better control for a very thin area of desiccation/coagulation. Because the patch material reinforces the cauterized edges of the biopsy site and/or tract, the use of bipolar RF electrosurgery is better.

In some embodiments, a frequency and wave type of RF electrosurgery for use in biopsy, hemostasis, and procedures related to this device are high voltage discharges, blended type waves and/or coagulation type waves, but depend on the specific procedure, location and type of electrodes used, and other factors that are dictated by the patient's specific needs. Blended cycles may be utilized, which is able to make the action faster than pure cautery mode. This is critical to preventing mechanical damage to the tissue in contact with or near the needle and/or other portions of the device. Ideally the once activated the action of cutting the biopsy would and any hemostatic prevention would be performed in less than 5 seconds, and even more preferable less than 3 and/or 1 second. This speed is also important to the user, and slowing down the procedure keeps them from other important activities. In some instances, the blended model RF electrosurgical waves are able to work faster than the pure coagulation model waves, but still able to they are able to adequately desiccate and alter the target tissue, preventing bleeding even from high pressure blood vessels (even when the patient's normal coagulation system is altered by medications or disease), turbulent flow, and diseased tissue. It also still optimizes the tissue at the molecular level for sealing and adhering to polymerized patch material, and photo-polymerized patch material by causing desiccation and protein denaturization without creating steam or exploding cells. The need for higher levels of adhesion to certain patch material may also make this wave mode preferable in some instances. The waves of these blended RF modes can be modulated (interrupted) waves, may be continuous, may have low to high duty cycles, may contain low to high voltages, and low % to 100% duty cycles (which is the percentage of time voltage is present over the activated period).

In some embodiments, the RF electrosurgery wavemode would be the pure coagulation mode. These waves typically have a lower duty cycle time than pure “cut” mode or “blended mode” type RFs. They are typically interrupted discharges, with higher voltages but for shorter periods of time than the other types. They also take longer time period to complete their action. This wave type may be preferred for certain types of diseases, tissue conditions, coagulation problems, electrode type, and/or type of patch material. The need for higher levels of adhesion to certain patch material may also make this the preferred wave mode type in some instances. For example, patients with kidney disease who need a kidney biopsy are often on anticoagulant medications for related problems such as heart disease and arrythmias, and/or DVTs or Pes. When their medications are reversed, the reversal is often not completely reversed (FFP is a blood product given to reverse warfarin use, but only gets INR to 1.4 at best when INR is normally 1 and fully anticoagulated is 2). Liver disease patients that need Liver biopsy often have problems with coagulation as well, because their liver's do not always synthesize an adequate amount and/or proper ratio of coagulation factors. Some if not most patients in each of these groups may benefit from the coagulation type mode as opposed to the blended and cut modes, as the risks related to longer procedures due to this setting may be greatly outweighed by the benefits of better sealed tissue, better adhesion of tissue molecules to other tissue molecules, better adhesion to certain patch materials, and less reliance on normal coagulation systems but still capable of sealing high pressure blood vessels.

In other types of embodiments, the wavemode would be the pure “cut” mode. These modes typically have continuous low voltage waves, with little variation in the size or shape of the wave. This wavemode would be of benefit when a tumor has been identified as cancerous or desire to ablate some area of tissue before, during, and or after the biopsy procedure. This type of RF wavemode may be especially useful in breast tissue biopsies of other tissues during tumor biopsy. This type of wave form may be best used with a biopsy system that includes suction, so tissue is not damaged during the procedure.

In some embodiments there is one set RF electrosurgical energy wave type and use type. In other embodiments, the wave and energy type can be set for specific organs, tissue types, disease severities, medication related issues, need for specific types of adhesion to patch material, and/or other factors.

RF desiccated and/or cauterized tissue is relatively inflexible compared to normal tissue, which may make photopolymerized patch material an ideal substance when used in combination with RF electrosurgery. When exposed to an area of a significant of swelling, the pressure and shear forces will tend to crack and break a sealed area of tissue, pulling apart at continuous area of RF electrosurgery treated tissue. This can disrupt hemostasis, tissue separation, continuity of the seal, or expose an area of otherwise sealed off vessel, tissue, or substance. This is especially true if the swelling takes place within a cylinder, an arch, and/or any space confining area of tissue that has been treated by RF electrosurgery. Because photo-polymerized patch material does not rely on swelling to create pressure in the surrounding tissue to cause hemostasis, the swelling ratio is typically much smaller than that used in other types of hemostasis material currently in use, such as Gel Foam.

In monopolar you need the dispersing (non-active) electrode to cover a large area. Active electrodes with small tips are generally used for cutting (point, hook, narrow tip, bladed edge. Active electrodes with broad surfaces are used for desiccation and coagulation and hemostasis. A split-pad is a dispersive electrode may be used in some embodiments as a part of monopolar RF electrosurgery. divided into two, so an impedance measured at both dispersive electrodes should match, if it doesn't the device shuts off because one of the dispersive pads may be coming loose, which could cause thermal injury.

There are two types of heating taking place with RF, electromagnetic energy causes the anion and cation molecules within the cells to move towards the different poles, so it is turned into kinetic energy, which is then converted into thermal energy through friction. Less important is electrical resistance. In some embodiments, the strength of the sealed tissue would allow for a second needle and/or biopsy to enter the same plane. If there was overlapped biopsy cut axis in the same plane it would not disrupt the effects of the seal, because the new seal would overlap the previous. Instead of weakening the seal, this would actually strengthen the seal as the polymerized hydrogel would polymerize and seal to the previous cauterized tissue or polymerized patch material.

In some embodiments, the biopsy device uses bipolar RF desiccation and coagulation along with a mechanical blade to cut the biopsy tissue from the tissue be. The use of bipolar RF along with a mechanical cutting action may be beneficial because vessels can be sealed consistently with the precise sensing of temperature and/or impedance measurements of a bipolar RF system, and the mechanical cut can then take place which causes less damage to the biopsied tissue bed and/or biopsy sample as opposed to an electro cautery used in cutting mode or some other type of cutting system.

In some embodiments, the bipolar RF electrosurgery active electrodes are blades themselves that are used to both desiccate and denature the proteins of the tissues of the biopsy sample connection to the biopsied tissue bed. This may allow for prevention of high pressure bleeding from occurring at the biopsied tissue bed. In some instances, the other bipolar electrode is a second blade. In some embodiments, the first bipolar blade is a portion of the exterior cutting needle, while the second bipolar blade is a portion of the core needle. This greatly simplifies the building of the device in an unexpected way and incorporates the prevention of arterial pressure bleeding while not significantly changing the motions or actions of the device. It also allows for prevention of thermal damage to the biopsy tissue and/or mechanical damage of the biopsied tissue and/or tissue bed during RF electrosurgical desiccation, coagulation, and and/or protein denaturing. This has the unexpected benefit of also minimizing the amount of tissue altered by RF electrosurgery, (when RF electrosurgery is the only type of hemostasis used) while still providing a tissue seal created by the denaturing and new bonds formed within the tissue itself that is capable of withstanding arterial level hydrostatic forces.

In some embodiments, the device uses the blade edges that are cutting the biopsy sample free to act as the active electrode surfaces to bipolar RF electrosurgical desiccation, coagulation, and/or protein denaturing along with the use of photopolymerized hydrogel is some portion of the needle tract and/or adhesion to the RF electrosurgery altered tissue bed. This provides the unexpected benefit of reinforcing the tissue bed denatured proteins. In some embodiments, this allows for absolute least amount of tissue to be altered by the RF electrosurgically sealing the tissue. First, the use of the electrodes in the biopsy blade edges minimizes the width of tissue exposed to the current. It also may allow precise sensing of the tissue to be cut to free the biopsy sample. Because the patch material also reinforces the biopsied tissue bed, the degree of tissue alteration can also be minimized.

In some embodiments, the device that uses RF electrosurgery with one, two, or more electrodes in the selectively at the cutting blade edges, would use RF electrosurgery frequency type of coagulation type frequency and/or blended type frequency. These frequencies are preferably modulated (interrupted) wave forms, which are coagulation type frequencies. Blended type would include frequencies that are modulated low-voltage waveforms, typically with wider duty cycles than the purely coagulation settings on the ESU. This also allows for the separation of tissue or biopsy sample from biopsy tissue bed to be mechanical and not caused by thermal means, while having the mechanical action be precisely aligned with the area that is monitored by impedance and/or thermal measurements. Using these frequencies as opposed to “cutting type frequencies” decreases the likelihood and size of altered tissue, decreases the volume of thermal tissue damage, decreases the likelihood of mechanical damage of tissue and sample due to exploding cells and rapidly expanding gases, decreases the likelihood of mechanically and/or thermally altering the biopsy sample which may destroy the utility and readability of the sample, increases the likelihood of the sealed tissue resisting arterial pressure hydrostatic forces and turbulent bleeding, and creates a surface with more bonding sites and stronger adhesion to patch material.

the tissue where the biopsy sample is cut from may cause the majority of major hemorrhages in most types of biopsies. Using patch material in the axis of the biopsy needle tract along with RF electrosurgery to specifically seal the biopsied tissue bed has the unexpected benefit of sealing the patch material selectively with the strongest bond to the biopsied tissue bed while flexibly still sealing other areas of the needle tract. The tissue that has been altered by RF electrosurgery has more mechanical bonding sites; with drier surface and more exposed hydrophobic molecules, more denatured proteins that allow for penetration into the tissue and entanglement, more hydrogen bonding sites among several others). That altered tissue bed also allows more chemical bonding sites and less water molecules obstructing chemical bonding sites. And that tissue also allows for better self-healing of the patch material bonding to the altered tissue bed, because of the stronger mechanical forces and more available low energy bonding sites that can form spontaneously. The bonding of the patch material to the other portions of the needle tract will be less as strong, though still provide strong hemostasis in those areas non-altered areas of needle tract in some embodiments as well. So, if there was movement or forces of the tissue affecting the tissue tract that were strong enough to breaking the seal of the patch material from some portion of the tissue in the biopsy needle tract, the biopsied tissue bed (where most major bleeding takes place) would have much lower likelihood of breaking its seal.

In some embodiments the biopsy device uses a combination of bipolar RF along with a laser cutting system. The bipolar RF can precisely desiccate and denature the tissue, which paired with laser can be quite advantageous. The device can precisely determine when the tissue in contact with the device is ready to cut with the laser. The laser use can be continuous, pulsed, or interrupted. But because the impedance and temp can be precisely taken, the laser use can be optimized to prevent overheating, which might otherwise damage the tissue biopsy tract, biopsy tissue bed, and/or biopsy tissue sample. This tissue would be at high risk of overheating when using both, but the precise control allows their use safely together. RF has the benefit of also better controlling the severity of tissue alteration, which can be used to minimize the thermal damage to the biopsied tissue.

Another unexpected benefit of denaturing the tissue before injecting patch material, especially with use of photopolymerized materials, is the reduction is changing the electrostatic forces of tissue altered during the procedure. Few experts in the practice of surgery or performing biopsy know the electrostatic charge of normal tissue is negative and that it naturally electrostatically repels most types of photopolymerizing molecules, monomers, and polymers. There is really only one monomer, chitosan, which is positively charged. When used with just photopolymerized patch material chitosan is electrostatically attracted to the tissues. This is because the cells walls of animals are typically negatively charged phospholipids. When these cells are destroyed as the molecules are altered and dehydrated during RF electrosurgery (and other types of heating), the negative charge of the tissue becomes much more positive, making most other types of patch material, especially photopolymerized patch material much more mechanically adhesive to tissue, before and after polymerization.

The “cutting mode” wave form mode that produces better coagulation for hemostasis. This is a low voltage continuous or non-continuous wave form. This prevents higher temperatures in the tissues from building up, but can still be used to measure impedance of the biological material between the electrodes exposed to tissue.

In some embodiments, monopolar type electrodes can be used alone or in combination with patch material. In some embodiments, this patch material is a plug or a mesh. In some embodiments this patch material is a photopolymerized substance. In some embodiments the patch material used in combination with the needle does not require photons for a polymer to form. In some embodiments, all or a portion of both the core needle and external needle are used as the active electrode in a monopolar device along with patch material. In some embodiments, this may provide a more consistent amount of tissue change throughout the entire area exposed to the active electrode than other types of electrosurgery, thermal, or acoustical types of cautery. This in turn, increases the likelihood to consistently adhere patch material to those areas, especially when used in conjunction with photopolymerized patch materials. This is because other types of electrosurgery and cautery may be difficult to achieve consistent results because of vascular structures, nerves, variations in tissue or vasculature, variations in liquids, variations in electrolytes, and/or some other variables which may affect tissue changes with more variability than monopolar RE. For example, when using RF electrosurgery in a bipolar mode with only the two cutting edges of the biopsy needle serving as the two active electrodes, the tissue and/or substances between those two portions may include the interior of a blood vessel which has lower impedance that the other portions of the substances in between the electrodes. This may cause an uneven cauterization effect during the RF electrosurgery activation, such that some portions of the activated area would not be altered in an optimized fashion (with percentage of water removed and/or percentage of molecules denatured varying significantly between portions of tissue. This would also extrapolate to patch material forming at different rates because of those differences, which would further increase the likelihood of hemorrhage and complications of the procedure. Because the non-active portion of the electrode is a significant distance from the monopolar active electrode, there would be an unanticipated constancy of that effected tissue, especially with regards to the changes that increase the speed and adhesive strength formed between the patch material and tissue or substances.

In some embodiments, it is a portion of the core needle that serves as the active electrode in the monopolar system, activating and altering the tissue at one, two, or more of the following locations: the distal tip of the needle, the most distal 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm. In some embodiments one, two, or more portions of the exterior cutting needle serve as the active electrode in a monopolar RF electrosurgical cautery system. In some embodiments, the active electrode is a portion of the inserted components that only comes in contact with the tissue and/or only activates during one, two, or more, portions of time the device is within the body. In some embodiments the monopolar active electrode includes portions of both the core needle and cutting needle, another portion of the needle or device. In some embodiments, portions of the needle the distal portion of the core needle and distal portion of the cutting external needle are used for biopsy that may be the same area exposed or different areas exposed to biological material. In some embodiments, the amount of exposed needle is circumferential on all or a portion of the needle. In some embodiments only a portion of the cutting external needle or some other portion of the device acts as the active electrode.

In some embodiments, the non-active electrode used in RF electrosurgery contains only one electrode to distribute the charge. Because of the relatively large size of the electrode, and the area in electrical contact with the patient, the tissues, cells, and substances near that electrode do not significantly increase in temperature during the procedure. In other embodiments, there are multiple electrodes used to distribute the charge of the non-active electrode. In those embodiments, the two, three or more non-active electrodes are separately attached to the ESU, such that the ESU can detect changes in the bioimpedance between the different non-active electrodes to detect any portion of area reduction related to the area of the patient, which may otherwise because a small enough area to heat that tissue, material, or substance. In some embodiments of the device there is a heat barrier that surrounds all or a portion of the area around the biopsied tissue, patch material, wiring, photon transporting portions of the device, or photon projecting portion of the device.

Another unexpected benefit of using the combination of RF radiosurgery to dehydrate, cauterize, vaporize, or otherwise alter tissue in combination with patch material, is to prevent the patch material from embolizing into or through blood vessels to an unintended location within the patient, which may be in a vessel, tissue, or organ adjacent, near, or relatively far away from the intended site of use. This is especially true for biopsy procedures, which may unexpectedly expose patch material to turbulent, venous, capillary, arterial, luminal, high pressured, anticoagulated, Arteriovenous malformations, or other types of dangerous blood flow. The blood flow which causes embolization of patch material may be unseen because of limitations of imaging equipment, such as x-rays, CT scans, MRIs, and ultrasound. For example, these vessels which can cause embolization of patch material may be significantly smaller than 1 cm in diameter. This is the standard width of an ultrasound probe beam, and vessels smaller than this size may not be detected or properly displayed on imaging device, which may prevent the users of a biopsy, endoscopy, bronchoscopy, or other device from accidentally entering or disrupting that structure before releasing patch material. Various similar limitations exist with the imaging equipment such as difficulty identifying, location, or differentiating the following: vascular structures and structures in the brain which provide a blood brain barrier, small vessels in the lung, vascular malformations and small high pressure vessels in the kidney, milk ducts in breast tissue, small arteries in the prostate, gastric and esophageal varices during endoscopy, tissue layer differentiation issues, and various other problems related to imaging quality which increase the likelihood of embolized patch materials.

Using RF electrosurgery before, during, or after the use of patch material or hemostatic agent in some embodiments of this device, significantly reduces the risk of embolization of these substances. Used in combination there is less exposure of patch material, it can reduce the likelihood of embolization caused blood flow of various types; normal blood flow, turbulent, venous, capillary, arterial, luminal, extraluminal, high pressured, anticoagulated, or other types of dangerous blood flow. It can prevent embolism of patch material caused by limitations of imaging in various type of tissues. It can allow use for otherwise prohibited types of patch material. It can even make patch materials currently is use safer and more effective.

This effect of RF electrosurgery preventing embolization of patch material is especially true when it cauterizes certain portions of the needle tract during some procedures, such as the portion of the needle tract from which a biopsy sample is cut or otherwise removed from the tissue. Cauterizing the tissue at the level or location of the biopsy can greatly reduce the risk of embolizing patch material. For example, a standard tru-cut style biopsy needle cuts the biopsy sample from a tissue bed about 1 cm proximal to the distal end of the needle, and biopsy sample cut is about 1.5 to 2 cm in length. Use of RF electrosurgery to reduce bleeding over all or a portion of these areas is especially helpful at reducing the likelihood of patch material embolizing. In some embodiments, only a portion, only half, only a quarter, or multiple segments of tissue are altered by RF electrosurgery in combination with patch use to decrease the likelihood of embolization, while decreasing the area damaged or altered. This segmentation of area may be especially important in diseased tissue that is unable to regenerate properly, such as tissue in the spine or brain, kidney, lung, eye, or other organs or portions of organs. This decreased amount of embolized patch material with use of some embodiments, gives the unexpected benefit of having much more precise control over the amount of patch material at the intended site. This is especially important when the patch material needs to prevent bleeding from high pressure vessels but expansion of the hydrogel may cause adjacent ischemia. This is true for certain organs such as the kidney or the brain. If the brain cortex, for example, has even a small amount of hemorrhage adjacent to a procedure, there is often an area called the penumbra, which is an ischemia or infarcted tissue that develops around the area of bleeding and appears as a thin encircling shadow. Having too much patch material in that area of brain tissue can cause too much pressure as well, especially if the material is hydrophilic. Preventing embolization of patch material with the combination of RF electrosurgery and patch material is also critical to precisely controlling the amount of patch material at the target site.

In some embodiments, the use of RF electrosurgery in combination with photopolymerized patch material has the advantage of further decreasing the likelihood of problems related to embolization to an unanticipated degree. The photopolymerized patch material will only reach the excited energetic state where it is exposed to certain wavelengths and intensities of light, but turbulent flow and high wetness, often cause the photopolymerization to selectively occur away from the tissue bed or lumen of needle tract. But because of the decreased blood and fluid flow, and denaturization of the molecules allows a higher percentage of the patch material monomers to come in contact with and bind to the tissue walls, the rate which the photopolymerization occurs in the walls of the tissue bed or lumen of tract can be greatly increased, further decreasing the likelihood for embolization. The photons can also preferentially be used to target the walls, which would not otherwise be possible with need from building a polymer center of the lumen then towards the tissue, to essential push against any bleeding which polymerizing. In some embodiments, the combination of RF electrosurgery and photopolymerized patch material therefor prevents the loss of patch material monomers and unbound polymers to embolization, which in combination to the photopolymerized gel's low swelling ration, allows for very precise amount of patch to be used, and can prevent multiple problems when used in sensitive tissue such as the cortex of the brain. This improved safety is also further protection for use of photopolymerized hydrogels in patients with high blood pressure, abnormal or diseased tissues, or problems with coagulation.

In some embodiments, the RF electrosurgery used to dehydrate and denature the tissues decrease the likelihood of embolization of non-photopolymerized patch materials to an unanticipated degree. Decreased amounts of bleeding, decreased turbulent flow, decreased wetness, and decreased hydrostatic pressure increase the rate or mechanical and chemical adhesion to target tissues and substances, as well as allow for polymerization to be specifically target the interface of the tissue tract or biopsy bed lateral walls without having to build the polymer from the center axis towards those tissue to build pressure to resist bleeding. It can also allow for three dimensional structures of bonding and polymerization to form, which can provide better prevention of embolization during healing and molecular reorganization, even 1, two, or more days after placement. This also increases the adhesive strength and burst pressure, and the rate at which those properties increase. These materials rely on photons of specific wavelengths and intensities for activation so they are much less likely to cause problems if they do happen to embolize.

In some embodiments of the device, the patch material is entirely activated by heat generated by the device. In some embodiments, the heat that activates all or a portion of the patch material is from the heat of the device, needle, cartridge, or some other aspect of the device. In some device there is a control to select the activation of the patch material by heat. In some embodiments, the control of this activation is automatic. In some embodiments, the activation occurs secondary to a semi-automated process, sensor, or other method.

In some embodiments, RF electrosurgical alterations of the tissue take place with one, two, or more portions of the inserted aspects of the device that serve as an active electrode, which are separated by one, two, or more portions of the inserted device which are part of the active electrode and do not conduct electricity to the tissue, materials, or substances. This may be accomplished using a physical barrier that does not allow conduction. This may be a portion of the inserted device that conducts electrons much slower, has a higher resistance or impedance, or is separated from other aspects of the device and/or tissue, materials, or substances in such a way that it does not transfer electrons to some other location. In some embodiments, these areas may be altered or changed. In some embodiments, the user is able to select which mode they want to use for the entire procedure and/or a portion of the procedure.

In some embodiments, the RF electrosurgically altered, acoustically thermally altered, cauterized, or otherwise altered tissue has skipped sections of unaltered tissue breaking up otherwise continuous sections of tissue in one, two, or more planes, when used in conjunction with polymers. Though these tissue can have high burst pressures and general strength, their low elasticity increases the likelihood of fracture under shear or twisting forces. Such factures can cause immediate or delayed bleeding or loss of seal. The fractures sections can also no longer withstand axial extension or compression, further unsealing and damaging underlying tissue. When skipped sectioning is used in conjunction with patch material, the more elastic patch material can allow for flexibility during shear and torsional forces, preventing fracture of the altered or cauterized tissue while maintaining high burst pressures, significantly exceeding those of elevated blood pressure above 140 mmHG, 200 mmHG, or more. This also allows for compression and extension of these sections greatly decreasing the likelihood of unsealing tissue under those forces. This structure combination also increases the ability for the patch material to self-heal. For example, when used in conjunction with a kidney biopsy, horizontal skip junctions can be placed between vertical potions of altered sealed tissue with respect to the axis of the needle tract. In doing so, there are different portions of the kidney tissue which may themselves have different elastic moduli, so body movement may create lateral or shear forces on just a small portion of the sealed track. This may damage a portion of the patch material, as it allows flexibility with that movement but concentrates its movement to the skip sections, especially the patch material on the side of the force occurred which would stretch as opposed to compress. So that side of patch material may crack, but likely only a portion and not the entire circular section of patch material in that section. Then when the force subsides, the broken edges of patch material would be held firmly together by the heat sealed portions of tissue, allowing mechanical bonds to reform as well as low energy chemical bonds, allowing self-healing of those substances while continuing to protect the tissue and organ against high pressure or anticoagulated bleeding.

In some embodiments, the patch material can be heated or cooled within the device, within the needle, during injection, or after injecting onto, within, or near the target location. In some embodiments, this increased the temperature of the patch material, reducing its viscosity and decreasing the force required to expel the patch material from the device. In some embodiments, the patch material is able to help reduce, decrease, increase, or otherwise alter the temperature of tissue or substances at a target location.

In some embodiments, the thermal changes in the patch material cause the local activation of the patch material. In other embodiments, the thermal effects used for tissue alteration can partial or fully polymerize all or a portion of the patch material. This action may be used in conjunction with photoactivation or alone, and may be used in some embodiments to improve the ‘toughness’ of the patch material within the device, near a certain portion of the device, proximal or distal location within the device, as the patch material leaves the device, during activation of the patch material, or some timing or location, or some combinations of these.

In some embodiments of the device, the changes in the tissue at the target location are induced by components of the device comprising ultrasonic vibrational cautery or tissue changes. In these devices, many of the same advantages apply as described above regarding tissue alteration by RF electrosurgery. However, the embodiments utilizing ultrasonic cautery may comprise a wireless cautery and instrumentation device, which may allow for easier control by the user. In some embodiments, the ultrasonic vibrational cautery device may have optional combined use of biocompatible patch material. This combination of ultrasonic cautery and biocompatible patch material allows the device to optimize the tissue targeted by the patch material, which can greatly improve the tissue seal strength as compared to either ultrasonic cautery or patch material alone. Ultrasonic cautery can be used to alter the temperature, viscosity, toughness, adhesive strength, bond strength or other aspects of the biocompatible patch material. In some embodiments, the biocompatible patch material used in conjunction is photopolymerized patch material, non-photopolymerized patch material, some other plug, mesh, substance, or some combination thereof. In some embodiments, the cautery and patch material are able to selectively be activated, changed, automated, semi-automated, or controlled in some other ways, or a combination thereof, depending on the comorbidities or risk factors of the patient. In some embodiment, the use of ultrasonic cautery along with release and activation of patch material allows for reduction in risk of complications. In patients with low or impaired coagulation factors, have low or altered platelets, are severely ill, have severely diseased tissue or organs, low RBC or hemoglobin count, or a combination of these problems, the reduction in risk related to the use of ultrasonic cautery in combination with patch material sealing may allow for otherwise contraindicated patients to receive a particular procedure, surgery, or therapy. In some embodiments, the patch material is a dual network photopolymerized gel used along with ultrasonic cautery to significantly reduce the risk of major hemorrhage, hemorrhage from arterial sources or arterial pressured vasculature, reduce healing time, reduce the required toughness or viscosity of the patch materials, reduce the volume radius locations or extent of tissue transformation related to cautery of the target tissue, other benefits, or a combination thereof. In some embodiments, the use of ultrasonic cautery in combination with patch material may allow for improved sealing of large, unseen, or anomalous vasculature.

In some embodiments the ultrasonic cautery delivering components heat the tissues in a controlled fashion to temperatures similar to that of RF electrosurgery, about 60 C to 100 C. In other embodiments, the ultrasonic cautery increases the internal temperature to selectively alter the patch material. In some embodiments, the device uses an oscillating electronic circuit to provide feedback, measurement, computations, activation, change, and control of the ultrasonic cautery components. In some embodiments the type, wavelength, frequency, or temperature generated by the device is controlled by the user, is automatic, or semiautomatic, and may be customized or optimized for one or more certain diseases, tissues, organs, coagulation abnormalities, locations, patch material, or other factors, or a combination of these.

In some embodiment's utilizing RF electrosurgery, ultrasonic cautery, or other tissue altering mechanism along with patch material, the temperature, bioimpedance, optical detection and measurements, other measurements, or a combination of these may be used to optimize the tissue for patch material adhesion, general hemostasis, arterial bleeding, tissues, organs, other affects.

In some embodiments, thermal transformation biological substances (RF electrosurgery, acoustic heating, cautery or heating of various other types, or cooling of various types) in conjunction with patch material and/or plug placement to seal wounds against high pressure bleeding after instrumentation, the patch material may act as a thermal sink. In some embodiments, this patch material acts as a thermal sink in the area surrounding the biopsied sample within the device. In some embodiments, the entire metallic needle portions, a portion of the needle, or multiple portions needle (s) are thermal conductive. The patch material which has not been expelled from the needle or device will typically not be in direct contact with the sample. But in some embodiments, the patch material is thermally well conductive, as are selectively all or a portion of the walls of material surrounding the biopsied sample holding section of the device. In some embodiments, after absorbing the heat (or providing heat) from the biopsied sample, the thermally conductive portions of patch material are circulated back to a larger volume of patch material or other thermal sink to better distribute this thermal change. In some embodiments, the heating or cooling of the area surrounding the biopsied tissue affects the chemical or mechanical bonds within the material, such that properties of the patch material are altered. In some instances, this thermal change decreases the viscosity of the patch material, which is beneficial for patch material with large viscous monomers such as polysaccharides, glycoproteins, or other molecules. These temperature changes can provide an unexpected benefit, by greatly reducing the force required to expel the patch material from the needle, and, in some instances, allow the patch material to better conform to the tissue defect. The temperature the patch material is raised to is to a temperature more the approximately 38 C but less than about 50 C, such that the viscosity is reduced but the tissue is not further altered by the patch material.

In other embodiments, the patch material has the benefit of reducing the temperature of the cells around the needle tract after being exposed to a thermal transforming force. There is the risk of some portion of the tissue receiving a faster removal of water or structural change. The electrical impedance of this area can be accidently increased in that scenario, and other portions of the tissue or biological substance receive a much higher conductive power increasing the temperatures to an unsafe level. Expelling thermally conductive patch material in the heated or cooled area of tissue would have the near immediate effect of normalizing those temperatures to a safe degree. It would be especially help in tissues such as the kidney and lungs that cannot regenerate, so it could prevent bleeding even at high pressures, while protecting against excessive thermal damage to remaining tissue.

In some embodiments, the patch material extruded from the needle provides the unexpected benefit of preventing the RF altered tissue from adhering to the inserted portion of the device.

In some embodiments of the device, the portion of the device which hold the biopsied sample after removing it from the biopsy tissue bed has flexible internal walls. In some embodiments which contain a flexible internal wall of the biopsy sample holding area, the lumen which carries the patch material will pass this flexible section of material though a continuous lumen, causing the flexible area to flatten and significantly increasing the cross-sectional area of this portion of the needle, thereby greatly reducing the required force and work performed to pass this section, possibly less than a quarter of that required in an unmodified biopsy needle. In some embodiments that contain a flexible backing to the area when the biopsy sample is stored, the patch material itself can be drawn back before, just before, at the time or, or during the biopsy, such that the negative force in the lumen of the column containing the patch material can transfer a negative pressure onto the tissue, and thereby a pull a section of biopsy sample into the cutting path and increasing the volume of the biopsy sample. In the same device, the size of the lateral defect that contains the biopsy sample can be reduce while still obtaining similar sample sizes with each use. In some embodiments, there would be a flexible external potion of the device that covers the external biopsy sample area, which may be a portion of the external cutting needle or a separate piece of the device. If again in direct connection to the column of patch material and internal flexible biopsy wall, this has the unexpected benefits of applying pressure directly to the biopsy tissue bed when the patch material pressure within the injecting column is increased, making more volume for patch material to pass through, decreasing the force require for patch material injection, and making the patch material more likely to adhere to the biopsied tissue bed, and more likely to stop bleeding of pressures above 140 mmHG.

Vessels are not compressed (Coapted) with use of RF cautery from a needle so the patch material is helpful at increasing their burst pressure tensile modulus:

Another reason why sealing the tissue with both patch material as well as RF electrosurgical coagulation and desiccation, is because large vessels or high pressure vessels damaged during biopsy cannot be coapted and compressed before sealing with RF applied from the biopsy needles. Large diameter or high pressure vessels will typically only be sealed by compression if these vessels along with some dispersed RF coagulation with low voltage continuous waveform energy. The coagulated tissue would have greatly increased likelihood of prevented hemorrhage with the combination however, because the energy of the vessels forces and pulsations can be better distributed throughout the surrounding tissue, and the

Burst pressure of patch material delivered to a biopsy site or needle tract after sealing is approximately in the range of 100 mmHG to 200 mmHG.

### V. Modular System

In some embodiments, the tissue repair device is provided as a modular system. In some embodiments, the modular system comprises a central unit. The central unit may allow for some components of the system to be disposed of to maintain sterility while retaining other components within the central unit.

In some embodiments, the central unit comprises a light source. In some embodiments, the central unit comprises at least one computer processing system. In some embodiments, one or more sensors are provided within the sensor unit. In some embodiments, a biocompatible patch material is loaded into the central unit. In some embodiments, the biocompatible patch material is contained within a cartridge. In some embodiments, a cartridge containing the biocompatible patch material is loaded into the central unit. In some embodiments, the biocompatible patch material is specific to the target tissue type. In some embodiments, a cartridge containing the biocompatible patch material is labeled with the target tissue type.

In some embodiments, a modular system comprises a light source provided outside of the central unit. In some embodiments, a light source is provided within a needle or instrument. In some embodiments, the biocompatible patch material is provided within a disposable. In some embodiments, a portion of the biopsy equipment is disposable. In some embodiments, all of the biopsy equipment is disposable. In some embodiments, a portion of the biopsy equipment is reusable. In some embodiments, all of the biopsy equipment is reusable.

In some embodiments, the modular system allows for substance to be advanced through needle before insertion to prefill instrument. In some embodiments, the modular system connects to biopsy equipment, vascular equipment, electrocautery equipment, surgical equipment, a needle, endoscopic equipment, and combinations thereof.

In some embodiments, a modular system is provided for 3D printing biocompatible structures. In some embodiments, a modular 3D printing system comprises a removable nozzle for expelling biocompatible material. In some embodiments, a nozzle is selected based on the shape, location, or tissue type of the biopsy site. In some embodiments, a modular 3D printing system comprises a plurality of interchangeable nozzles.

In some embodiments, the modular system allows for selection of certain patch material with predetermined type of tissue altering component, such as ultrasonic cautery, RF electrosurgery, thermal resistive cautery, freezing, some other tissue altering mechanism, or a combination of these things.

In some embodiments, the modular system allows the user to separately select the tissue altering components and patch material type, or controls thereof, to optimize the device for specific goals, tissues, or risk factors. These selections may be simplified in some embodiments, so the user can simply choose predetermined combinations to meet certain criteria of target locations, or a dial switch or control to selectively turn on or off different actions or components. In some embodiments there are more complex selection capabilities, such as cartridges of different patch material to meet different needs of target tissue or organ location, disease type or severity, density diameter or size of local vasculature to target location, cartridges for different tissue sealing mechanisms, special wires and pads selectively used for the monopolar cautery actions, select device alterations for dampening of tissue altering effects, of other issues or a combination of these things. In some embodiments, the user is able to select one or more patch material or patch material component or additive for photoactivated or non-photoactivated substances, including but not limited to concentration of substances, additives and curing time may also be adjusted for specific target, organs, disease, or a combination of these factors. In some embodiments, all or a portion of device is preloaded with patch material or tissue altering mechanisms. In some embodiments, there are some reusable portions that may be cleaned, covered, sterilized, autoclaved, of otherwise treated to prevent transmission of disease while also reducing the cost of the product or its components.

In some embodiments, the device comprises a method of using a modular system biopsy and patching system such that disposable cartridges can be used for biopsy capture, mixing patch material, partially polymerizing the patch material within a lumen of the device, delivering that partially polymerized patch material to a portion of the biopsy tract, and locking to prevent reuse of some portion of that disposable cartridge. In some embodiments, the device comprises a method of using a modular system biopsy and patching system such that disposable cartridges comprised of a biodegradable light guide can be used for biopsy capture, mixing patch material, partially polymerizing the patch material within a lumen of the device, delivering that partially polymerized patch material to a portion of the biopsy tract.

### 1. Method of Modular System Use in Biopsy

In some embodiments, the device comprises a method for instrumentation and wound sealing. Some embodiments are intended for use performing a biopsy procedure and preventing major hemorrhage by sealing the biopsy tissue tract. In some embodiments, the user selects one or more instrument cartridges comprised of one or more biopsy needles for specific biopsy procedures, an extendable waveguide, patch material cartridge attachment site, patch material component viewing window, attachment and attaches sites for attachment to the device housing. The user then attaches the selected instrument cartridge to the device handle, which comprises a handle, housing, plurality of controls, instrument cartridge attachment points, and instrument cartridge control components. In some embodiments, the user separately chooses a patch material cartridge according to one of more variables related to the target organ, disease process, disease severity, other factors, or a combination thereof and attaches that to the device housing as well (In some embodiments, the patch material cartridge comprises a portion of the instrument cartridge). In some embodiments, the user then connects the light source to the housing in sterile fashion. The user then presses the photopolymerization priming mechanism, which in some embodiments automatically runs system check, checks the viability of the patch material components, and causes the patch material components to travel to the distal needle tip, and mixes two types of monomers in solution with a photoinitiator component in the process. The user then uses a sperate or integrated imaging device to identify the target location, approach for instrumentation, and any hazards or other issues which can be seen. The user decides whether or not she intends to use the photopolymerized patch sealing mechanism (which would take place immediately after the biopsy procedure if toggled to the on position), and turns on patch mechanism activation using one external switch to toggle both the release and seal activation on or off. She then begins the procedure, which can be performed using standard techniques in some embodiments. When the distal end of the device is adjacent to and aligned which target biopsy tissue, the user then presses the biopsy activation button which activates the biopsy sampling mechanism, which is followed immediately by patch activation initiation within the distal lumen of the device so as to enhance strength and viscosity of the patch material to seal the tract even when the tract is bleeding at hypertensive arterial pressures (up to about 300 mmHG) and anticoagulated blood. The patch material is simultaneously compelled from the distal needle tip and activated with light while in contact the target tissue, while also retracting the needle tip(s) towards the handle of the device (allowing more intensity of the forming polymer column in line with the tissue biopsy tract). The activation process also triggers the automatic locking mechanism preventing repeated biopsy procedures, which is triggered by the photopolymerized patch use as well. The patch sealing mechanism continues over a time period of about 5 seconds and cures approximately the distal 3 cm of the biopsy tract. In some embodiments when complete, the removal indicator flashes and the needle can be safely removed from the patient. The user then presses the ablation step activation button which helps break down the photopolymerized gel to prevent sample contamination. The user then presses the biopsy sample retrieval button and removes the biopsy sample. The user can then attach a new instrument cartridge and repeat the process if they so choose. This is only one example of numerous possible embodiments, and the components, sequence, and procedure type can vary greatly while performing instrumentation and wound sealing.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term arteriostatic refers to an agent or substance which prevents bleeding (e.g., arterial, venous, and/or capillary bleeding) and hemorrhaging.

The section headings used herein are for organizational purposes only and are not to be construed as limiting the subject matter described.

### VII. Exemplary Embodiments

### Manually Operated Biopsy and Biopsy Site Repair Device

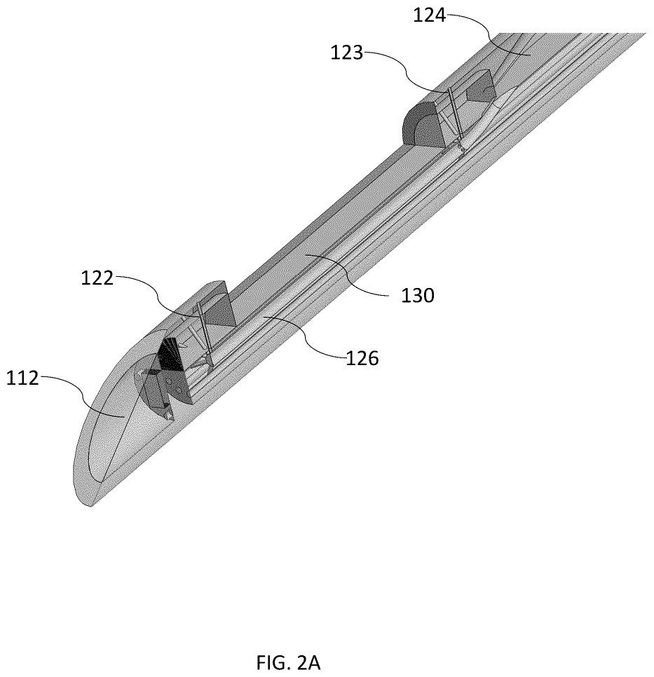

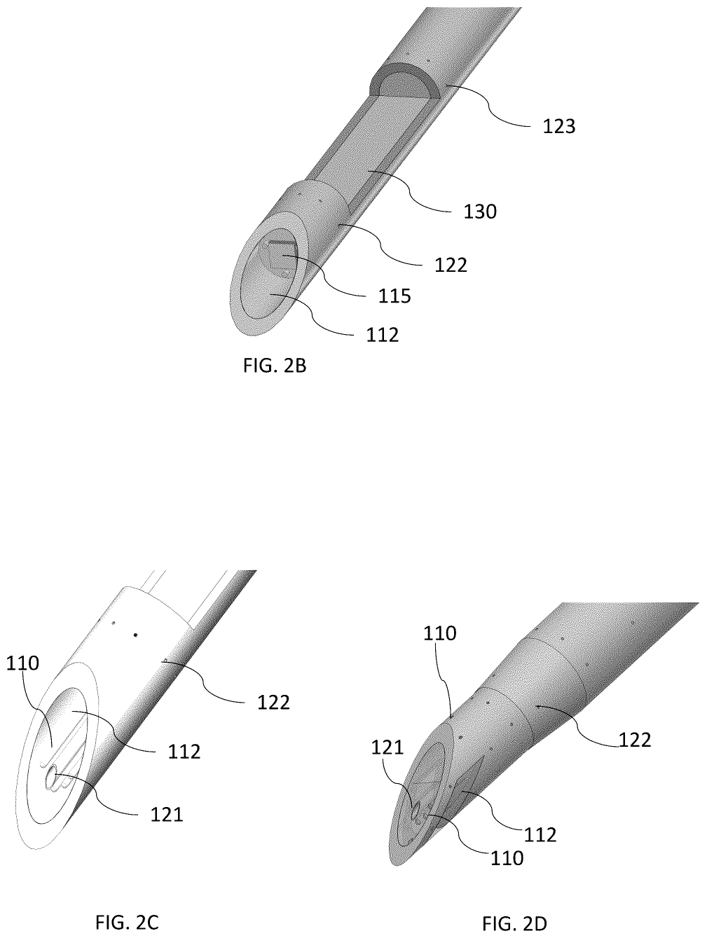

According to some embodiments, a tissue repair device and related components are depicted by FIGS. 1A-2D. In some embodiments, as depicted by FIG. 1A-1E, a tissue repair device may comprise a body 105 and a needle 120. In some embodiments, the needle 120 is a biopsy needle comprising a sample tray 130 for collecting a tissue sample. In some embodiments, the sampling tray 130 may be enclosed or covered by sheath 150. In some embodiments, sheath 150 is configured as a cutting sheath. In some embodiments, a shield 155 is provided to prevent contamination of the body 105 of the device. In some embodiments, the shield 155 may be manipulated to manually translate the sheath 150.

In some embodiments, the device comprises a reservoir 140 for containing a biocompatible patch material. In some embodiment, sheath 150 is translated proximally toward the body 105 to expose the reservoir and biocompatible patch material. In some embodiments, the needle is provided with one or more apertures 121, 122, 123 in fluid communication with a reservoir 124 of biocompatible patch material. In some embodiments, the reservoir 140 comprises an internal portion 124 which extends into the interior of the needle and/or body of the device. In some embodiments, a lumen 126 provides allows transfer of the biocompatible patch material through the device and throughout apertures. In some embodiments, a plunger 125 is provided to translate the biocompatible patch material through a fluid circuit and to a target area.

In some embodiments, the device comprises a light source 115. In some embodiments, the light source is housed within the body. In some embodiments, a light source is provided at a distal tip of the device. In some embodiments, one or more optical elements guide light from the light source to one or more light emitters 110. The light emitters may be provided within a lumen of a needle or on an exterior surface of the needle. In some embodiments, apertures 122, 123 are utilized as emitters. In some embodiments, activation of the light source, emission of a biocompatible patch material, translation of the cutting sheath, or a combination thereof is initiated by depression of a plunger or button by a user. In some embodiments, one or more light diffusing and/or transparent covers 112 are provided to prevent substances from entering a tip of the needle while allowing light to transmit to a dispensed patch material.

### Automated Biopsy Site Repair Device

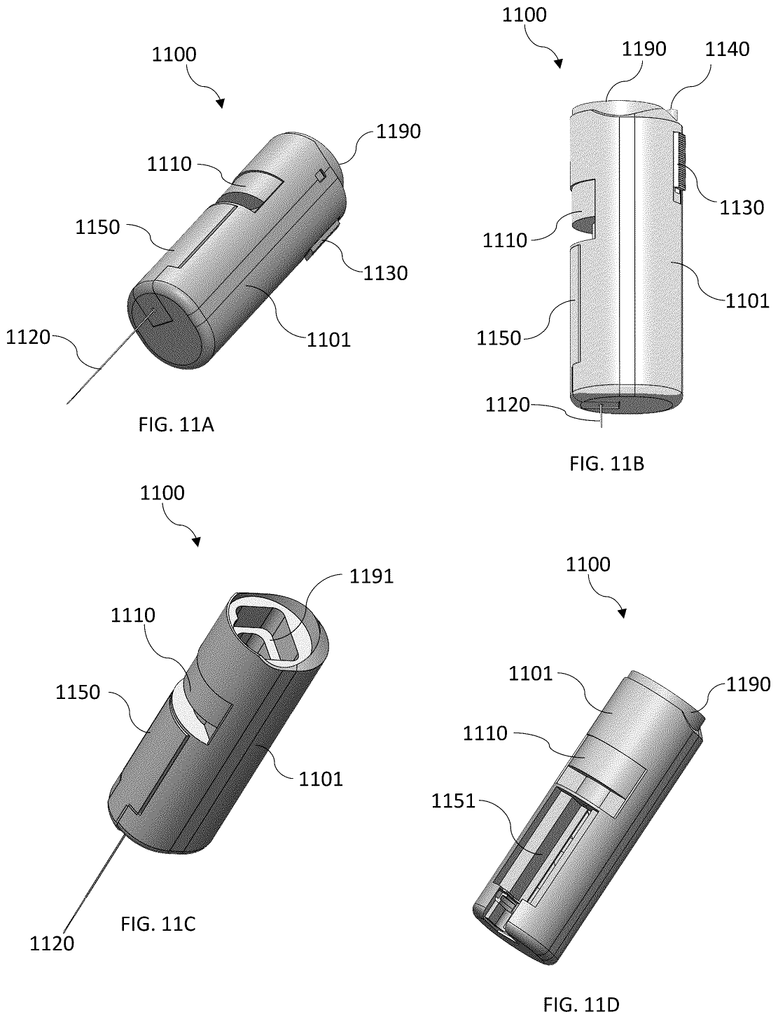

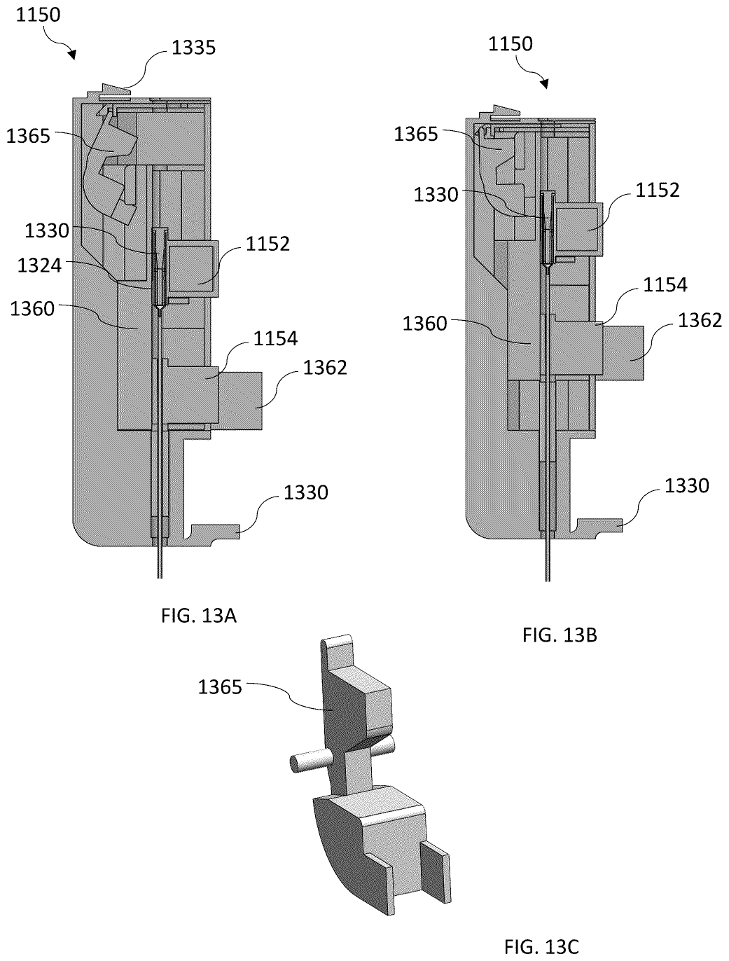

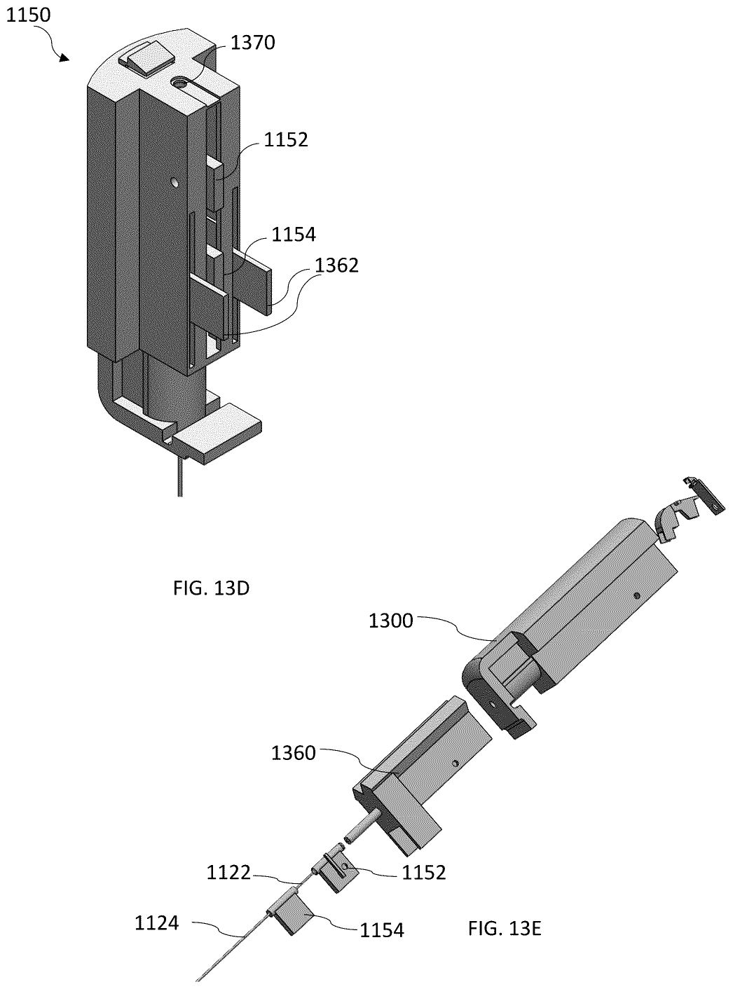

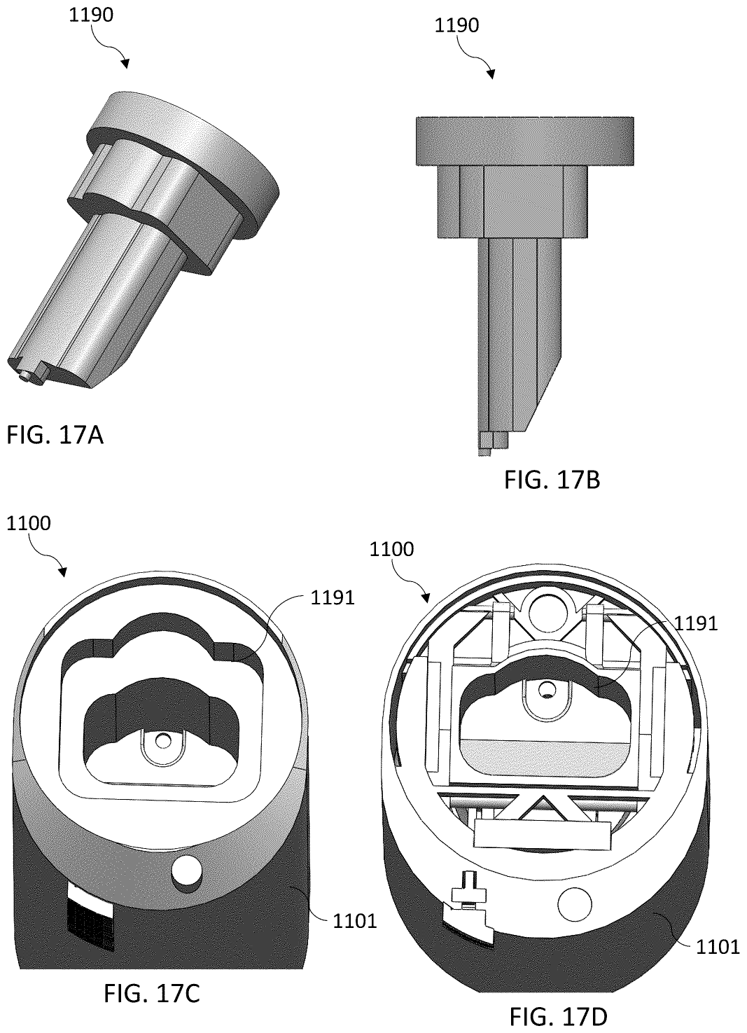

With reference to FIGS. 11A-17D a device for automated tissue biopsy collection and repair at the site of the biopsy is depicted, according to some embodiments. In some embodiments, a biopsy site tissue repair device 1100 is provided comprising a housing 1101. In some embodiments, the housing 1101 retains the components of the cocking mechanism for the device. The cocking mechanism allows for simple loading or cocking and subsequent activation of the device to take a tissue sample using biopsy needle 1120, dispense a biocompatible patch material, and emit light to cure said biocompatible patch material.

In some embodiments, the housing 1101 comprises a needle cartridge recess 1151 to removably receive a needle cartridge 1150. In some embodiments, a user first loads the needle cartridge 1150 into the housing 1101. In some embodiments, after loading the needle cartridge, the user engages the cocking trigger 1110 to activate the cocking mechanism. In some embodiments, the cocking trigger 1110 is engaged by moving it toward the proximal end of the device 1100. In some embodiments, the cocking trigger 1110 is engaged by moving it toward the distal end of the device 1100 The user may then insert provide the needle 1105 at the biopsy site and depress the activation button 1140. In some embodiments, depression of the activation button 1140 releases cocking mechanism. In some embodiments, wherein the needle 1120 is a biopsy needle, when the activation button 1140 is depressed to release the cocking mechanism, sample tray moves relative to the outer sheath of the biopsy needle, such that a tissue sample is cut from the biopsy site and retained in the sample tray. In some embodiments, depression of the activation button 1140 causes the biocompatible patch material to dispense from the distal end of the needle 1120 and into the biopsy site. In some embodiments, the device 1100 further comprises a light source 1190 which is placed within a light source recess of the housing 1191. In some embodiments, depression of the activation button 1140 causes activation of the light source 1190. In some embodiments, the light source 1190 is used to cure a photoactivated biocompatible patch material, as described herein.

In some embodiments, the needle cartridge 1150 comprises a housing 1101 proximal needle wing 1152 and a distal needle wing 1154. In some embodiments, the proximal needle wing 1152 is coupled to an inner needle 1122 of a biopsy device. In some embodiments, the inner needle 1122 comprises a sample tray for collecting and retaining a tissue sample. In some embodiments, the distal needle wing 1154 is coupled to an outer needle or sheath 1124 of a biopsy device.

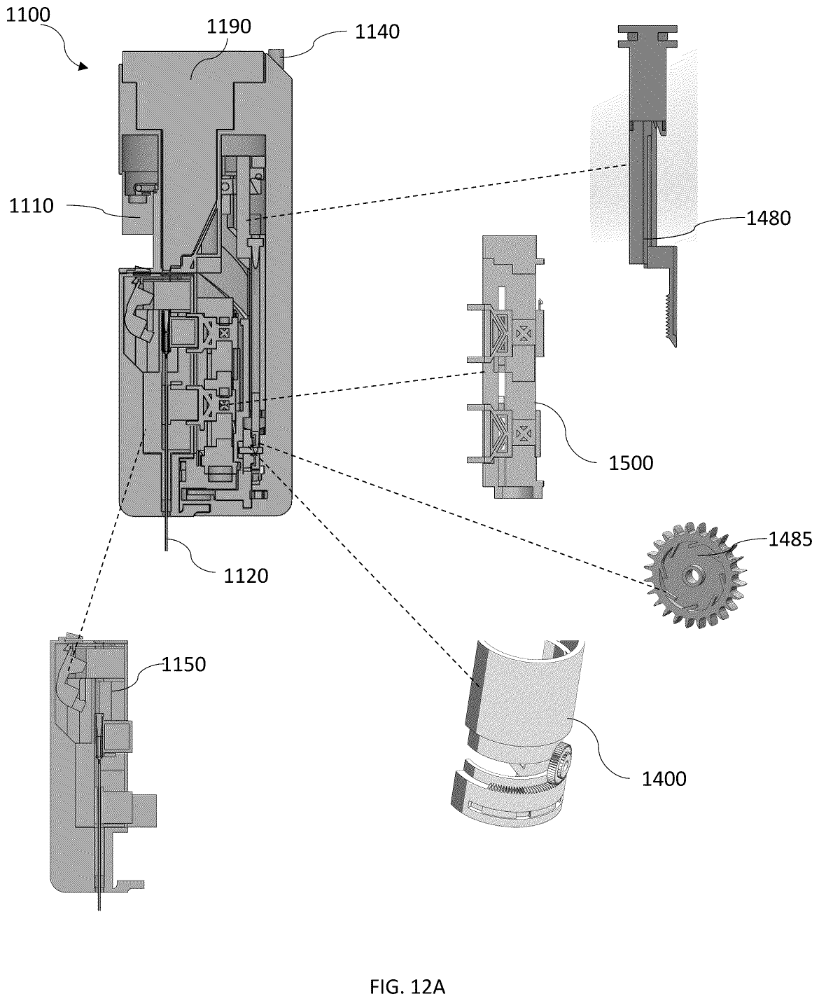

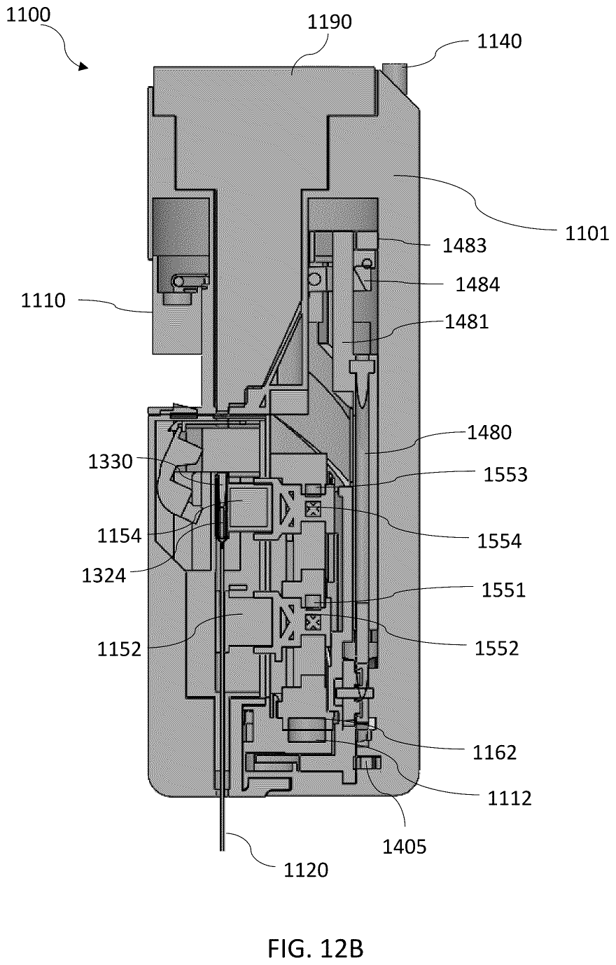

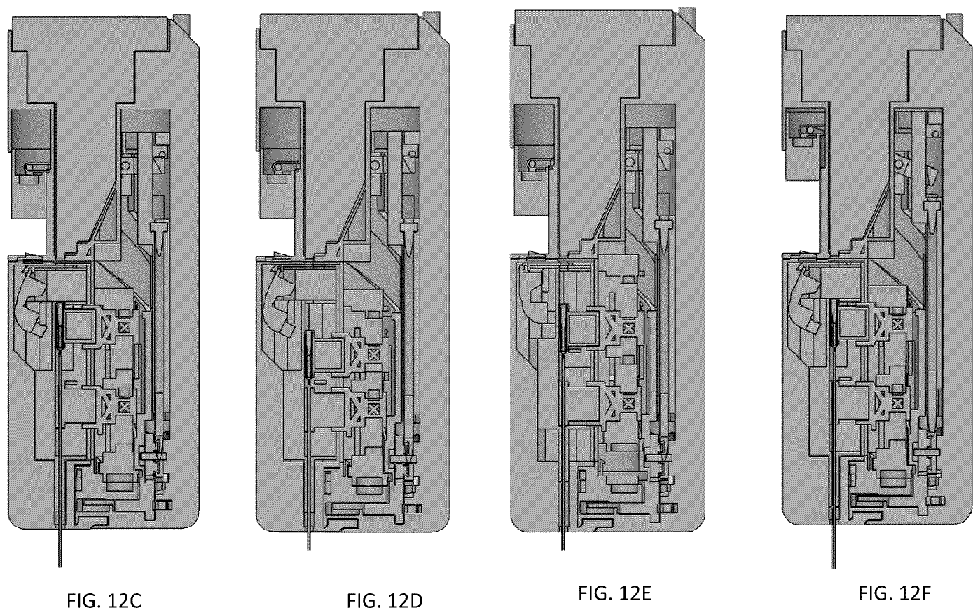

In some embodiments, when cocking trigger 1110 is pulled in the proximal direction, the proximal needle wing 1152 and a distal needle wing 1154 are provided in a proximal position and the inner needle 1122 and outer needle 1124 are in a retracted position. This may be referred to as the first position. The cocking action and first position are depicted in FIGS. 12C and 12F, according to some embodiments. Upon depression of the activation button 1140, the proximal needle wing 1152 and inner needle 1122 move distally, such that the sample tray of the inner needle is exposed from the outer needle or sheath 1124. This may be referred to as the second position and is depicted in FIG. 12D, according to some embodiments. Upon a second depression of the activation button 1140, proximal needle wing and the outer needle or sheath 1124 move distally, such that the outer needle or sheath 1124 covers the sample tray of the inner needle 1122 to enclose a sample. In some embodiments, this movement also causes cutting of the tissue sample, as disclosed herein. This position may be referred to as the third position. Upon a third depression of the activation button 1140, biocompatible patch material is pushed through a lumen of the needle and dispensed at the biopsy site. This may be referred to as the fourth position and is depicted in FIG. 12D, according to some embodiments. In some embodiments, this position also activates the light source to activate a photoinitiator of the biocompatible patch material. In some embodiments, cartridge 1150 can be filled with patch material via aperture 1370. In some embodiments, tab 1335 and edge 1330 mate with the housing 1101 to lock the cartridge 1150 into place.

With reference to FIGS. 12A-16F, further details of the automated biopsy and biopsy site repair device 1100 and its components are depicted, according to some embodiments. In some embodiments, the device 1100 comprises a carrier cage 1500 and a cocking plate 1400. In some embodiments, the carrier cage 1500 comprises proximal wing carrier 1554 and distal wing carrier 1552. In some embodiments, the proximal wing carrier 1554 is biased in a distal direction by a spring (not shown) abutting surface 1553. In some embodiments, the distal wing carrier 1552 is biased in a distal direction by a spring (not shown) abutting surface 1551 In some embodiments, the carrier cage 1500 is biased in a proximal direction by a spring (not shown) abutting surface 1512 of the carrier cage 1500 and surface 1112 of the housing 1101. As depicted, a recess may be provided to retain the springs.

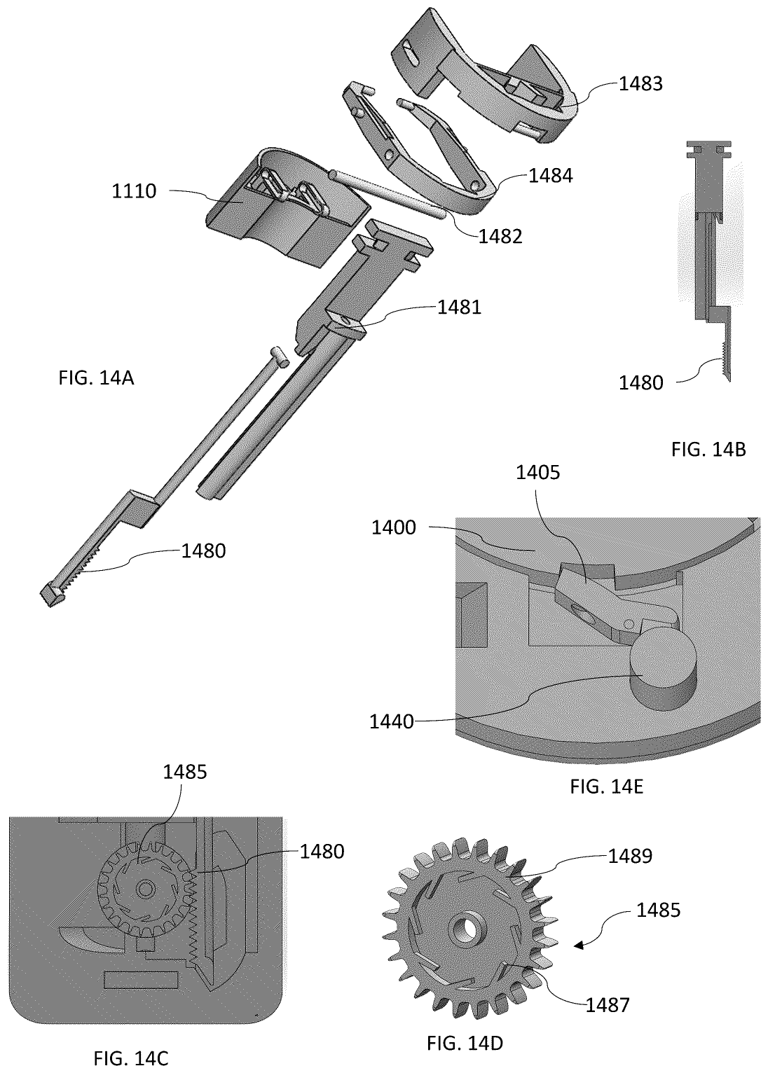

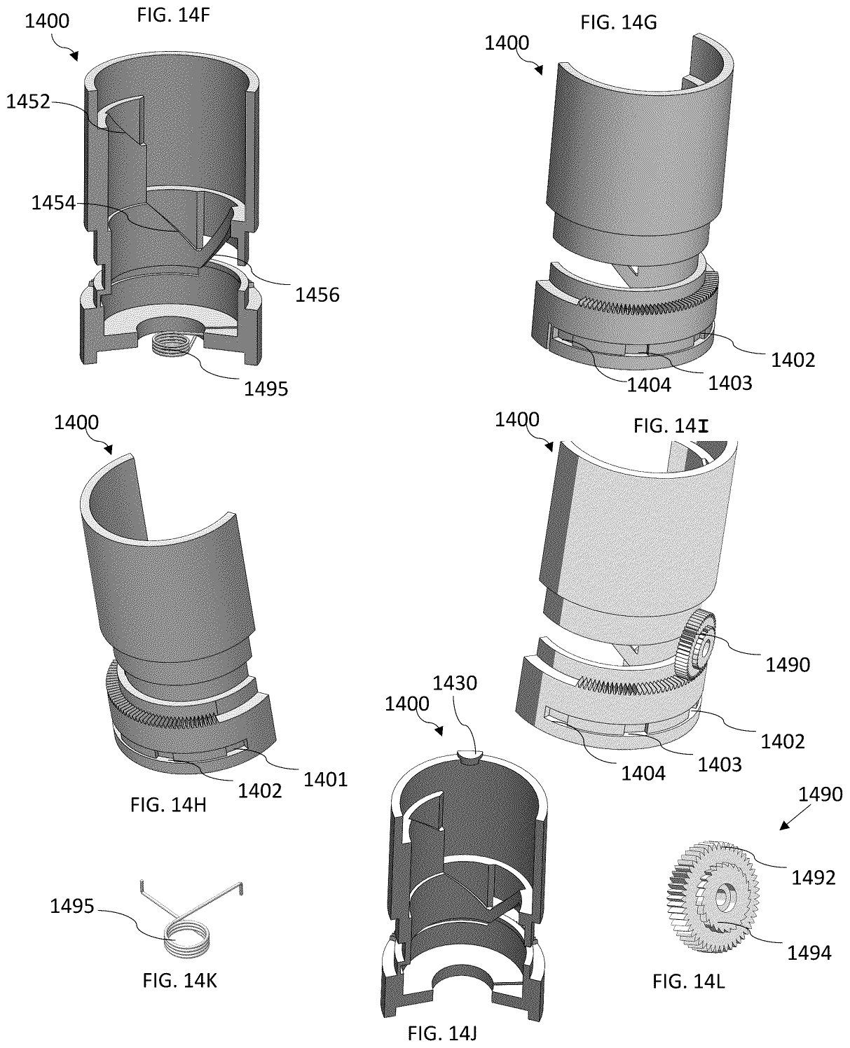

In some embodiments, the cocking mechanism comprises a cocking plate 1400. Cocking plate 1400 may be referred to as having a partially circular or annular shape. In some embodiments, cocking plate 1400 comprises a spring 1495 (depicted in FIGS. 14F and 14K) to bias the plate. In some embodiments, as depicted, the spring 1495 biases the plate 1400 in a counter-clockwise direction. In some embodiments, the plate comprises a plurality of teeth which engage with a cog 1490 (as depicted in FIGS. 141 and 14L). In some embodiments, the cog 1490 comprises an outer diameter 1492 having a plurality of teeth and an inner pawl 1494. In some embodiments, the inner pawl 1494 of the cog 1490 is fits within a gear 1485 comprising direction arms 1487 such that they form a one-way locking gear. In some embodiments, gear 1485 comprises and outer diameter with a plurality of teeth 1489 which engage with the teeth of cocking rack 1480. In some embodiments, the cocking rack 1480 is coupled the cocking trigger 1110 by cocking slide 1481, cocking pole 1482, cocking pull 1483, and cocking lever 1484. In some embodiments, the arrangement is such that when a user engages the cocking trigger, the vertical motion is translated through the cocking lever 1484 resulting in vertical movement in the proximal direction of cocking rack 1480. In some embodiments, the proximal motion of the cocking rack 1480 rotates gear 1485 and cog 1490, thereby rotating the cocking plate 1400 in a and creating a bias by spring 1495.

In some embodiments, the locking plate 1400 comprises four recesses 1401, 1402, 1403, 1404 which are engaged by a cocking pawl 1405. In some embodiments, the cocking pawl 1405 is biased into the recesses 1401, 1402, 1403, 1404 by spring (not shown) having one end retained within a recess 1405A of the cocking pawl 1405. In some embodiments, the cocking pawl 1401 is engaged by an extension of the activation button 1440 comprising a ramped portion, such that the pawl is withdrawn from a recess and the locking plate is allowed to rotate until the locking pawl engages in the next recess. In some embodiments, the arrangement allows for the rotation of through all positions if the activation button 1440 is held down by a user. In some embodiments, the recesses correspond to the four positions of the device, referred to herein, wherein recess 1401 corresponds to a first position just after cocking, recess 1402 corresponds to a second position after a first depression of the activation button, recess 1403 corresponds to a third position after a second depression of the activation button, and recess 1404 corresponds to a fourth position after a second depression of the activation button. In some embodiments, because the mating configuration of gear 1485 and cog 1490 provides a one-way pawl, the gear does not resist or translate the cocking rack 1480 as the locking plate 1400 rotates under the bias of the spring. In some embodiments, cocking pawl 1405 contacts the outer surface of the locking plate as it rotates. In some embodiments, this contact provides friction to slow the rotation of the locking plate after the activation button is engaged.

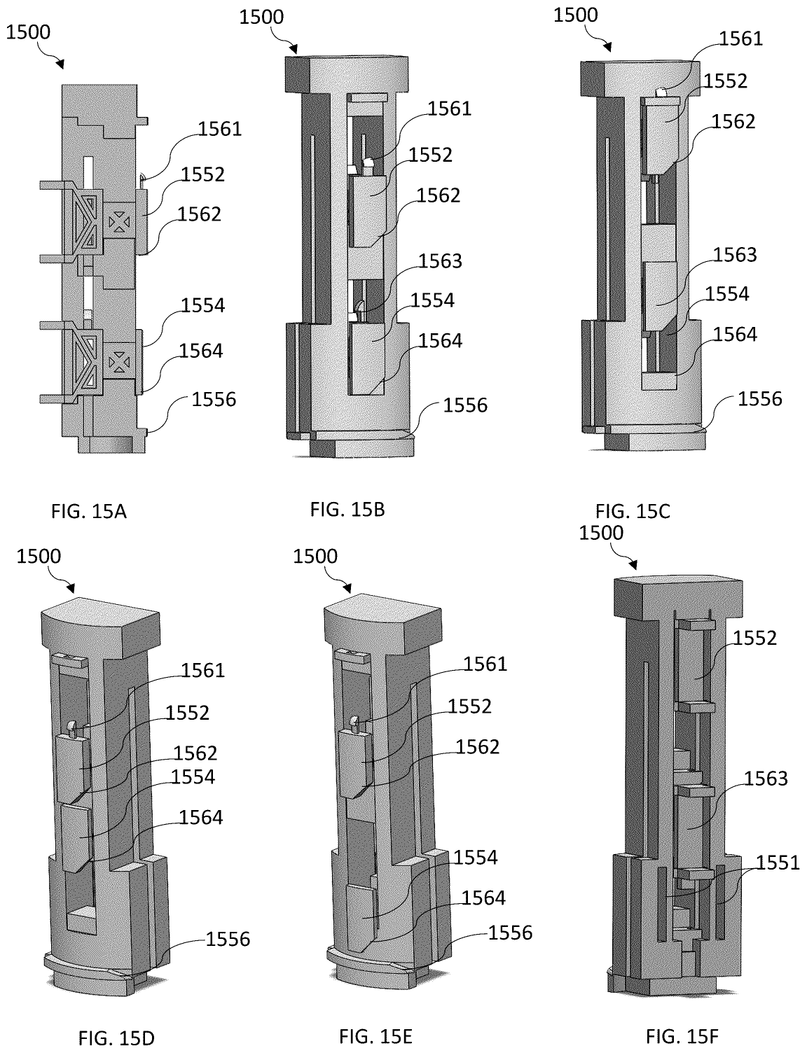

In some embodiments, the wing carriers 1554, 1552 comprises tabs 1563, 1561 which engage into recesses, (e.g., recess 1567 depicted in FIGS. 15D and 15E). In some embodiments, depression of the activation button engages a ramped portion of the tabs to release them from their corresponding recess.

The locking plate also comprises a distal wing carrier cam surface 1454 and a proximal wing cam surface 1452 to urge the distal wing carrier 1554 and proximal wing carrier 1552, respectfully, into the proximal position (against the bias of their respective springs) as the locking plate 1400 rotates during cocking of the device. In some embodiments, the corresponding abutting surfaces (1564, 1562, respectively) of the distal wing carrier cam surface 1454 and a proximal wing cam surface 1452 are depicted in FIGS. 15B-15E. In some embodiments, the distal wing carrier 1554 and proximal wing carrier 1552 couple to the proximal needle wing 1152 and a distal needle wing 1154, respectively, when a needle cartridge 1150 is inserted into the needle cartridge recess 1551 of the device 1100. Therefore, as the locking plate 1400 is rotated through its positions, the needle wings are positioned as described above.

In some embodiments, the carrier cage 1500 comprises a cartridge wing recesses 1562 for receiving patch actuator wings 1362 of the patch actuator 1362. In some embodiments, the carrier cage 1500 further comprises a lower extrusion 1556 which abuts the carriage cocking cam surface 1456 of the cocking plate 1400. In some embodiments, as the cocking plate moves beyond into the fourth position, the carrier cage is biased upward by the corresponding spring. In in turn, this pushes the patch actuator 1362 upwards, also retracting the needle assembly. When patch material is provided within the patch material storage cell 1324 of the needle cartridge 1150, the patch material is forced out through one or more aperture provided proximal to the distal end of the needle assembly, as discussed herein.

In some embodiments, upward movement of the patch actuator 1362, engages lever 1365 to open provide an aperture such that light emitted from light source 1190 can reach a light receiver. In some embodiments, a needle integrated light receiver 1330 is provided to receive light through the open aperture, from the light source, and act as a waveguide, transmitting light to the biological site being treated. In some embodiments, a light source activation protrusion 1430 is provided on the locking plate 1400 which, in turn, activates the light source for photoinitiation of a photopolymerizable biocompatible patch material.

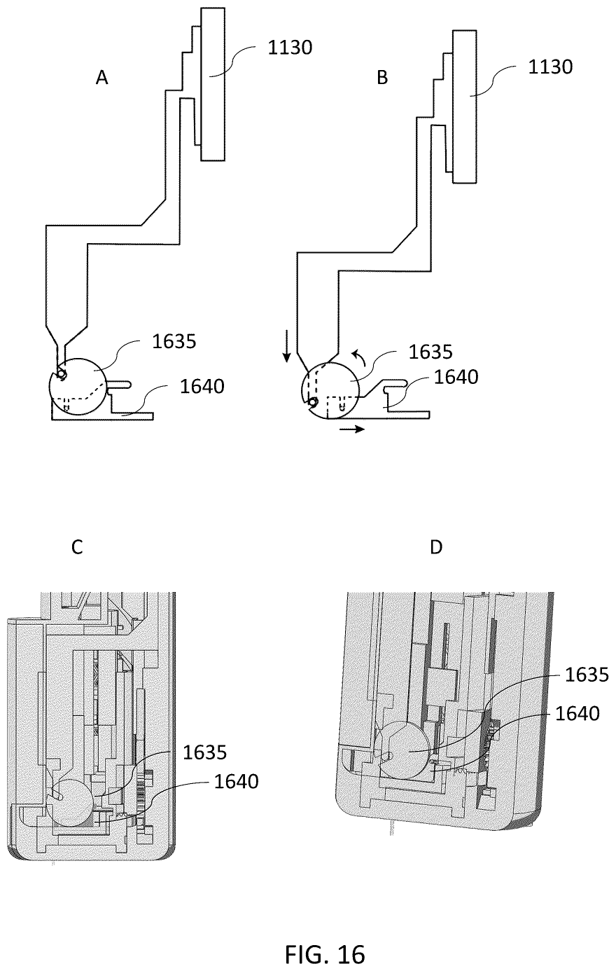

In some embodiments, the device further comprises toggle switch 1130. In some embodiments, the toggle switch 1130 is provided in a proximal position to allow a biocompatible patch material to be dispensed and for the light source to activate. In some embodiments, the toggle switch 1130 is provided in a proximal position to prevent a biocompatible patch material to be dispensed and for the light source to remain unactuated. In some embodiments, in a proximal position, the toggle switch will prevent the needles from retracting. FIG. 16A-16D depict toggle switch 1130, according to some embodiments. In some embodiments, sliding toggle switch 1130 to a distal position rotates toggle wheel 1635 and pushing lock 1640 over a bottom portion of the locking plate 1400, preventing the locking plate from moving upward due to the spring bias and thereby preventing the patch actuator from moving upward. In some embodiments, a top protrusion of the lock 1640 abuts the prevents the

One skilled in the art will appreciate that components of the device could be modified to create a desired result or procedure. For example, the needle cartridge could be modified such that the device provides tissue repair only and does not perform a biopsy. In another example, the needle cartridge may provide a biocompatible patch material which is not photoactivated, therefore it may be desirable to provide a modified needle cartridge which does not allow for activation of the light source and/or blocks transmission of the light source. For example, FIGS. 16A-C.

Further, one will appreciate that the device and components thereof could be further modified as described herein. For example, the needle cartridge could be provided with components necessary for cauterization, or mechanical components of the device could be replaced with electrical, pneumatic, and/or magnetic components. Further, mechanical delays may be introduced to modify the timing of the components. However, one will appreciate that the fully mechanical device of some embodiments may provide a lower cost per unit.

### Lever Action Biopsy Site Repair Device

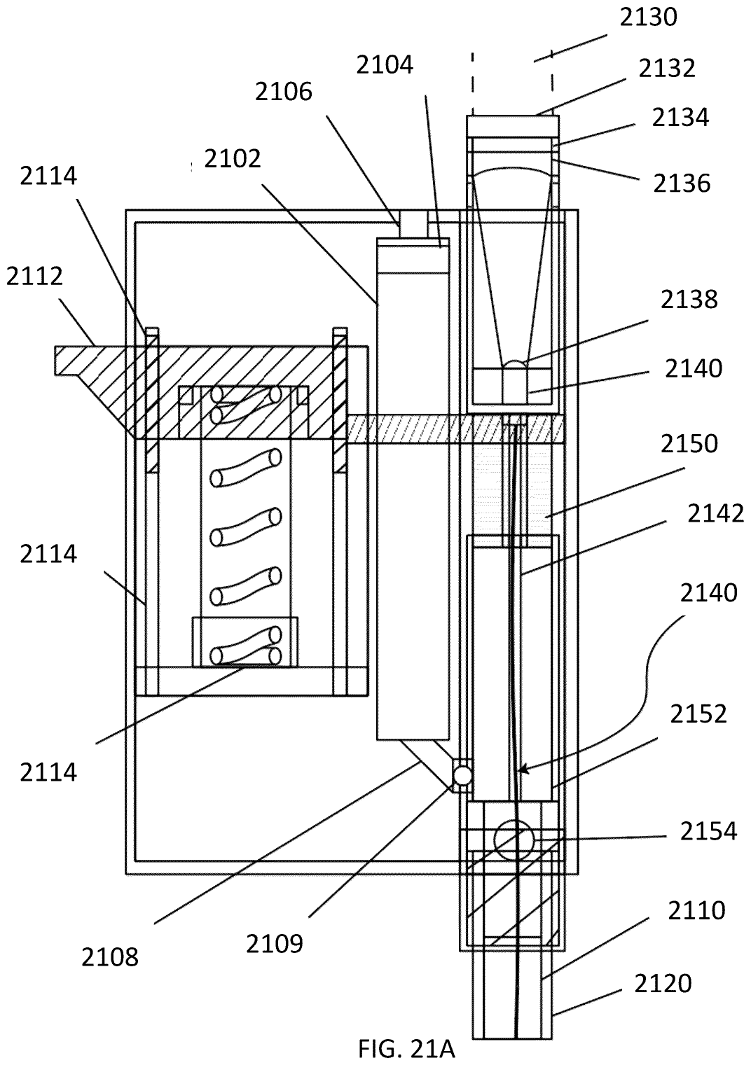

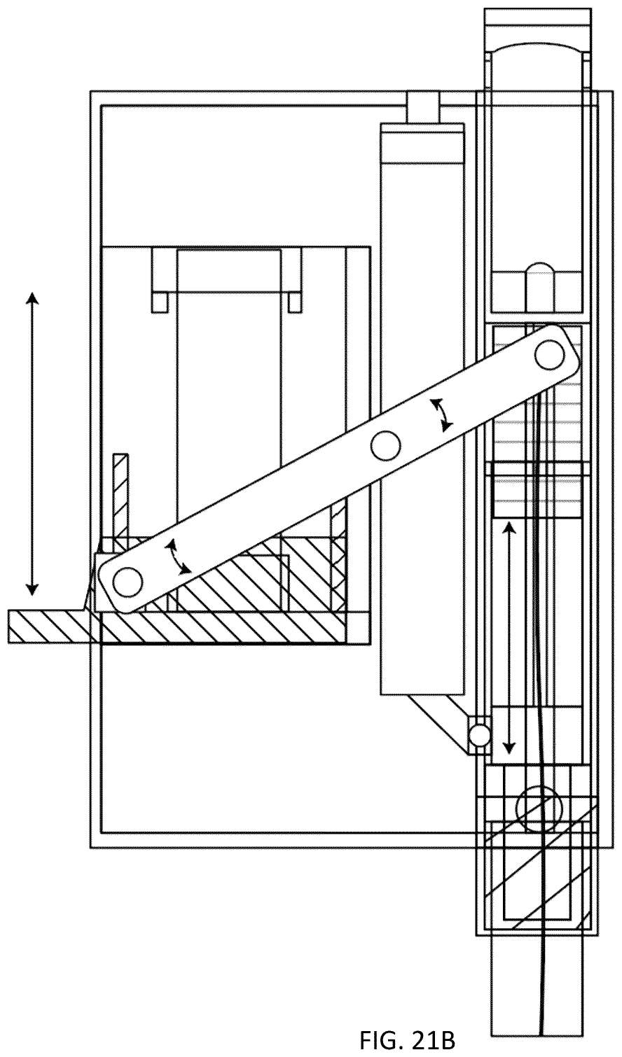

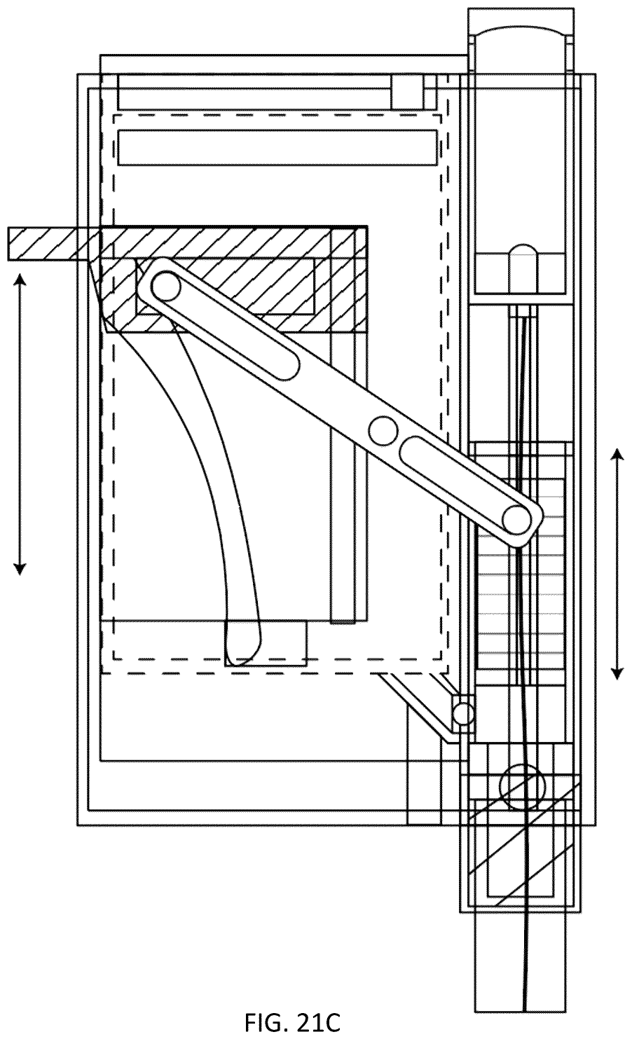

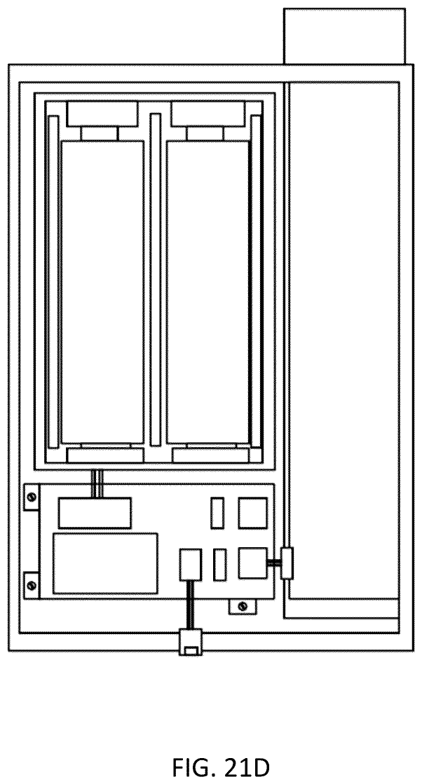

With reference to FIGS. 21A-21D, a biopsy site repair device is depicted, according to some embodiments. In some embodiments, the device comprises patch material storage chamber 2102 having a rear plunger 2104 and air vent 2106. In some embodiments, a tubular connection path 2108 provides the patch material in the storage chamber in fluid communication with a lumen 2010 of a needle 2120. In some embodiments, a one-way valve 2109 is provided along the tubular conduit 2108. In some embodiments, the device comprises a patch does delivery lever 2112 which translates along one or more sliding guide arms 2114. In some embodiments, the device comprises a patch does delivery lever rest 2116.

In some embodiments, the device comprises a light source 2130 for activating a photoinitiator of a patch material. In some embodiments, the device comprises a light receiver lens 2132 for focusing or collimating light. In some embodiments, a wavelength selection filter 2134 for filtering out wavelengths for the light source. In some embodiments, the device comprises a proximal beam concentrator 3136 and/or an internal light concentrator 3138 for guiding light into the proximal end of a waveguide 3140. In some embodiments, the waveguide 3140 is provided in a waveguide shaft 3142 directs light from the light source to a distal end of the needle.

In some embodiments, the device comprises a patch material plunger 2150 provided to move the patch material through the patch dose holding chamber 2152. In some embodiments, the lever is lifted up near the patch dose holding chamber to fill the chamber 2152 with the patch material (as depicted in FIG. 21B). The lever is then pressed down to push the patch material out of the chamber 2152, through a one-way valve 2154 and out a lumen of the needle 2120. In this manner, the desired amount of patch material may first be loaded into the chamber 2152 prior to dispensing through the needle lumen. FIG. 21D depicts a similar device comprising two material storage chambers, according to some embodiments.

### Light Conducting Catheter

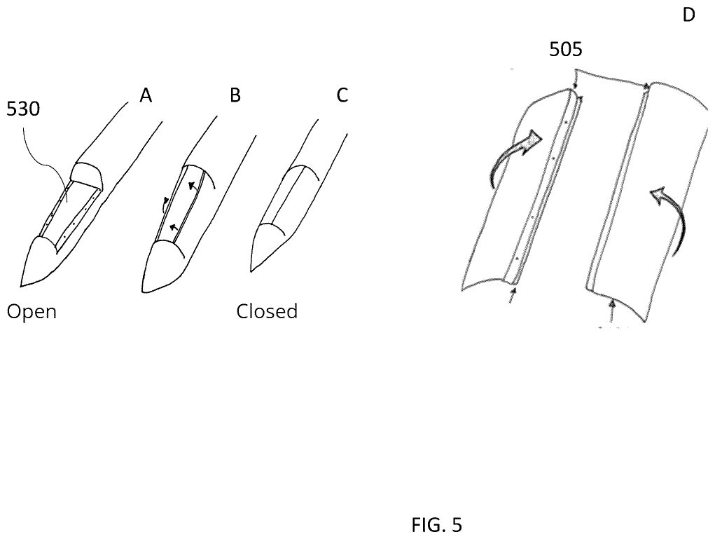

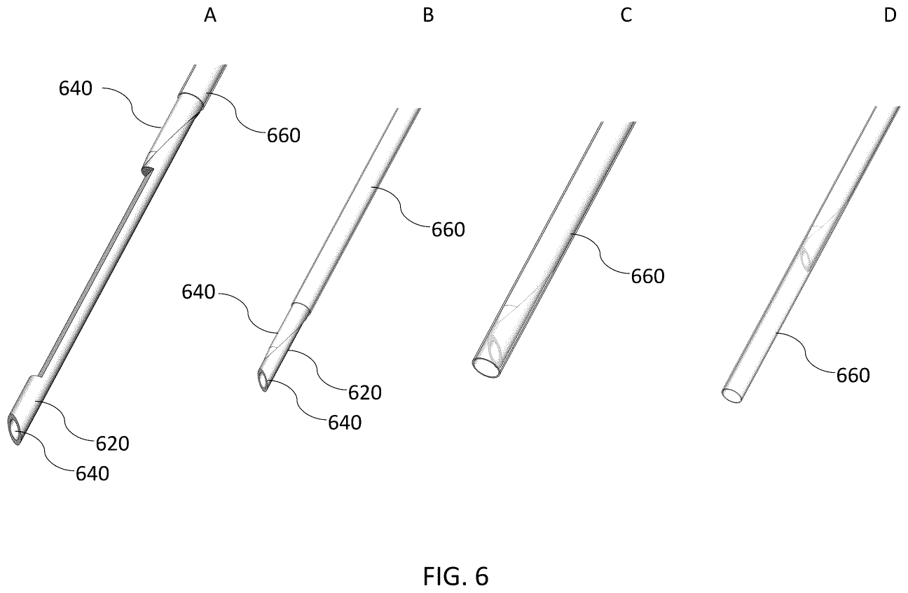

In some embodiments, a tissue repair device is provided to target location using a catheter. A light conducting catheter may be ideal for preventing injury to organs, and allows for prolonged activation of photopolymerized patch material without risk of injuring biological structures. A light conducting catheter is depicted in FIGS. 6A-6D, according to some embodiments. In some embodiments, the core and cutting needles 620, 640 are withdrawn into the light conducting catheter 660 and the light conducting catheter has flexibility so it won't harm the tissues, structures, or organs, but it has strength against piercing pressure from the needle, further protecting the tissues. In some embodiments, the core and cutting needles can be fully removed from the organ or tissue which is at higher risk for bleeding. Of great importance, this embodiment allows the user to have the regular flow of actions, while having the great benefit of patch material released to prevent high pressure bleeding, high volume bleeding, or bleeding in tissues, organs, or patients where normal coagulation mechanisms are impaired. In some embodiments this mechanism allows for the photoactivated gel to cure for 10 seconds, 30 seconds, 1 minute, five minutes, or thirty minutes safely, without risk of needle injury even with patient, device, and organ actively moving.

After the biopsy is advanced just after the biopsy is cut, such that is advanced to the tip of the core needle. Then the patch material begins to be ejected from the inner lumen of the core needle. The photopolymerized gel is ejected from the distal tip of the needle. The patch material within the light transducing catheter which keeps it taught at its distal end providing some amount of pressure against the walls. The walls of the light conducting catheter has fenestrations in some embodiments, such so it can release more patch material at the specific location of biopsy.

In some embodiments, the light conducting catheter acts as an electrical resistor as well. This allows for optimal amount of the distal end of the device to alter tissue at the distal 0 cm to 6 cm before the dispensing of the patch material. In some embodiments, the distal end of the needles (core and cutting) act as a monopolar electrode of RF electrocautery. This device allows the unique benefit of allowing the user to selectively activate zero, one, two, or more of the hemostatic measures while using the single device and simple controls. For example, a patient with new onset kidney disease without risk factors and with a normal coagulation system, may only require patch material (non-photopolymerized) to have nearly zero percent chance of major hemorrhage. A patient with advanced kidney disease has various factors which increase the likelihood for hemorrhage, many of which have not previously been identified. These patients are likely to need photopolymerized patch material deployed, along with the significant photocuring of that patch to reduce the risk of major hemorrhage to an acceptable level. Many patients who need live, kidney, colon prostate, brain, lung, or other types of biopsy have major problems with their innate coagulation system. They might also be at risk for other materials, liquids, substances, or air from leaking or contaminating unintended areas, such that these biopsies require both tissue alteration (such as monopolar electrosurgery) along with photoactivated gel release and cured seal. Embodiments of the present invention allows for all of those, again, without significantly changing the user experience.

In alternative embodiments of this type, this device could use bipolar electrosurgery, thermal cautery, freezing, acoustical cautery or some other form of tissue alteration or hemostasis, along with the release of patch material.

Of note, with the “light catheter embodiment,” there are some embodiments that have a second catheter surrounding the proximal end of the needle and light catheter. This serves multiple unanticipated purposes; because it can be used as a stable base to create friction with and holding position of the device relative to the tissue and organs automatically. This can be utilized to withdraw the light catheter at a specific rate so it can properly cure the photopolymerizing gel. This second catheter can also be used to shield the other tissues and biological substances from the photons, thereby allowing the device to only target the wound, or the patch material, or both. In some embodiments, there may be some photopolymerization occurring while the patch material is within the lumen of the light catheter after exiting the needle. This can allow the toughness of the patch material to increase before exposure to high pressure or turbulent bleeding. There can also be multiple apertures, diffusion areas, shielded areas releasing less photons, other components, or some combination of these things. In some embodiments the inner lumen of the device releases low levels of photons than the exterior portion, thereby promoting full polymerization at the location of the tissue.

The light catheter can also be used seamlessly in conjunction with cautery or other type of tissue alteration, combining that with photopolymerized patch material. The light carrying catheter, or another catheter, some other surrounding substance, or a combination of these can be used to dampen or prevent the tissue altering mechanisms from affecting tissue or substances outside of the target area.

The light carrying catheter can also be used to protect tissue from the sharp or blunt trauma by the biopsy needle tip in some embodiments. This can also have the benefit of allowing the light diffusing portions of device to safely stay within the patient, organ, tissue, or substance for a longer period of time, which is an unanticipated benefit improving both safety of the device and diversity of patch materials and device components.

With respect to the delayed/timed withdrawal of the light projecting element, this can be accomplished in many ways by various embodiments mechanically, electronically, magnetically, or otherwise. In some embodiments, it can use the viscosity of the patch material to resist a movement, there by automatically controlling the speed of withdrawal. For example, if there is a helical style pump screw is attached within the center lumen of the core needle, which is coupled mechanistically to both to the rotation of that patch driving force as the device as well as mechanical withdrawal of the light source and/or needle, so the rotation and ejection of patch material into a target location can also be used resist a driving force, such as a spring or motor, thereby automatically mechanically controlling the speed of the light source movement. The cartridge containing patch material can also adjust the rate of patch material flow or light intensity or motion of the light source, other things, or some combination of these.

### VII. Examples

The following examples are included for illustrative purposes only and are not intended to limit the scope of the invention.

### Exemplary Methods of Biopsy and Subsequent Repair of the Biopsy Site

FIGS. 41A-41F depict a biopsy and subsequent repair of the biopsy site using a biocompatible patch material and a balloon to provide compression at the biopsy site. The biocompatible patch material may be a photoinitiated patch material, as disclosed herein. One or more components of the devices may be configured as a waveguide to transmit light to activate the patch material. A lumen may be provided through the inner cannula of the biopsy needle. The lumen may be connected to a pneumatic device for inflation of the balloon. In some embodiments, light is transmitted through the balloon

In some embodiments, a first step is depicted by FIG. 41A, wherein a biopsy needle is beginning insertion into the biopsy site. In some embodiments, a second step is depicted by FIG. 41B with the inner needle of the biopsy needle, which comprises a sample tray, being fully inserted into the biopsy site. In some embodiments, a third step is depicted by FIG. 41C with the outer lumen or sheath of the biopsy needle moving to cover the sample tray and enclose a tissue sample. In some embodiments, a plunger pushes a biocompatible patch material as or after the sheath covers the sample tray. In some embodiments, the balloon begins inflation. In some embodiments, a fourth step is depicted by FIG. 41D with the balloon fully expanded such that it compresses the wound site and reduces bleeding as the biopsy needle is removed from the biopsy site. In some embodiments, the compression from the balloon facilitates adhesion of the patch material to the biopsy site. In some embodiments, a fifth step is depicted by FIG. 41E with the balloon beginning to compress. In some embodiments, patch material continues to be dispensed as the balloon compresses. In some embodiments, a sixth step is depicted by FIG. 41F with the balloon fully compressed and retracted within the distal end of the needle.

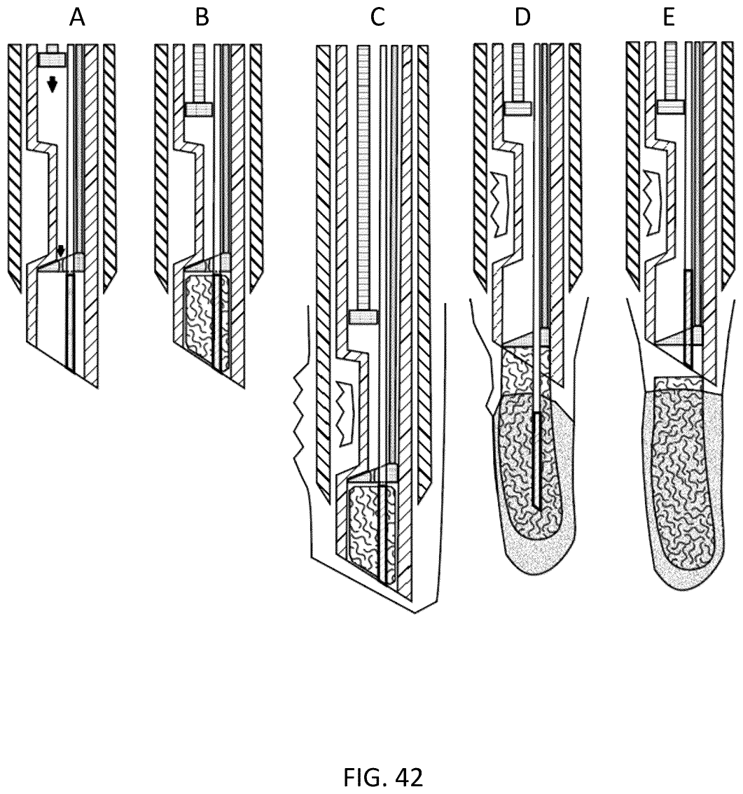

FIGS. 42A-42F depict a biopsy and subsequent repair of the biopsy site using a biocompatible patch material comprising a photoinitiator and an extendable light source.

In some embodiments, a first step is depicted by FIG. 42A, wherein a biopsy needle is beginning insertion into the biopsy site. In some embodiments, a second step is depicted by FIG. 42B with the inner needle of the biopsy needle, which comprises a sample tray, being fully inserted into the biopsy site. In some embodiments, a third step is depicted by FIG. 42C with the outer lumen or sheath of the biopsy needle moving to cover the sample tray and enclose a tissue sample. In some embodiments, a plunger pushes a biocompatible patch material as or after the sheath covers the sample tray. In some embodiments, a fourth step is depicted by FIG. 42D with the patch material being dispensed into the biopsy site and the extendible light source being left in the biopsy site as the biopsy needle and sample are removed from the biopsy site. In some embodiments, a fifth step is depicted by FIG. 42E with the extendible light source starting to be removed from the biopsy site. In some embodiments, patch material continues to be dispensed as the light source is removed. In some embodiments, a sixth step is depicted by FIG. 42F with the needle and light source fully removed from the biopsy site and retracted within the distal end of the needle.

FIGS. 43A-43E depict a biopsy and subsequent repair of the biopsy site using a biocompatible patch material comprising a photoinitiator and an extendable light source.

In some embodiments, a first step is depicted by FIG. 43A with a biopsy needle prepared for insertion into the biopsy site. In some embodiments, a second step is depicted by FIG. 43B with the patch material being dispensed and initiated prior to insertion of the device into the biopsy site. In some embodiments, a third step is depicted by FIG. 43C with the outer lumen or sheath of the biopsy needle moving to cover the sample tray and enclose a tissue sample. In some embodiments, a fourth step is depicted by FIG. 43D with the patch material being dispensed into the biopsy site and the extendible light source being left in the biopsy site as the biopsy needle and sample are removed from the biopsy site. In some embodiments, a fifth step is depicted by FIG. 43E with the needle and light source fully removed from the biopsy site and retracted within the distal end of the needle.

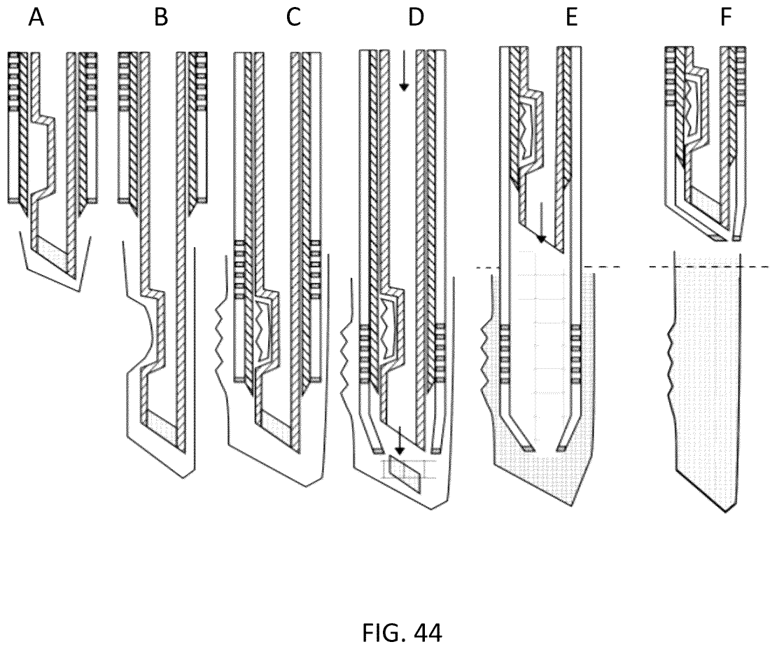

FIGS. 44A-44F depict a biopsy and subsequent repair of the biopsy site using a biocompatible patch material and a catheter.

In some embodiments, a first step is depicted by FIG. 44A, wherein a biopsy needle is beginning insertion into the biopsy site. In some embodiments, a second step is depicted by FIG. 44B with the inner needle of the biopsy needle, which comprises a sample tray, being fully inserted into the biopsy site. In some embodiments, a third step is depicted by FIG. 44C with the outer lumen or sheath of the biopsy needle moving to cover the sample tray and enclose a tissue sample. In some embodiments, catheter begins to move around the biopsy needle. In some embodiments, a fourth step is depicted by FIG. 44D with the patch material being dispensed into the biopsy site. In some embodiments, a fifth step is depicted by FIG. 44E with the patch material being dispensed into the biopsy site and the catheter being left in the biopsy site as the biopsy needle and sample are removed from the biopsy site. In some embodiments, a sixth step is depicted by FIG. 44F with the needle and catheter fully removed from the biopsy site.

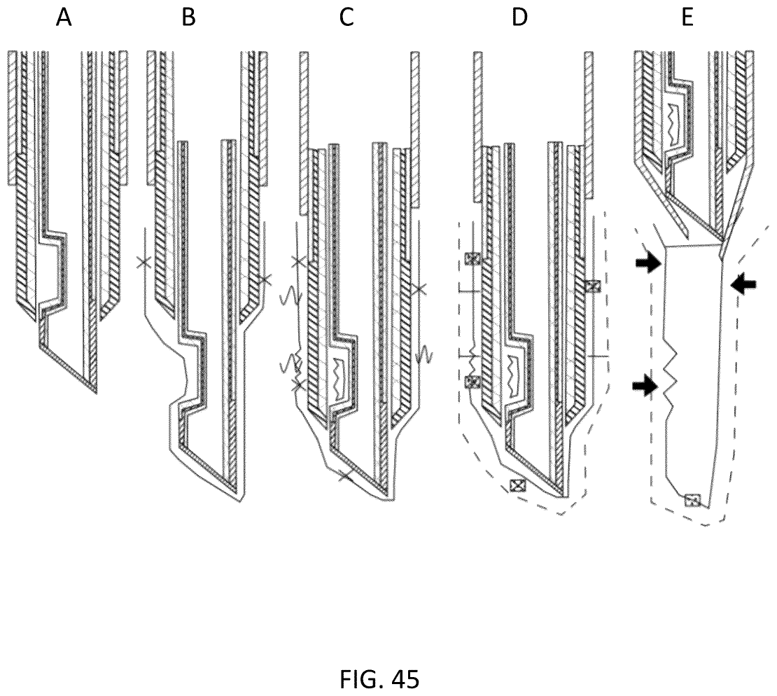

FIGS. 45A-45F depict a biopsy and subsequent repair of the biopsy site using a biocompatible patch material comprising a photo initiator and a light transmitting catheter.

In some embodiments, a first step is depicted by FIG. 45A, wherein a biopsy needle is beginning insertion into the biopsy site. In some embodiments, a second step is depicted by FIG. 45B with the inner needle of the biopsy needle, which comprises a sample tray, being fully inserted into the biopsy site. In some embodiments, a third step is depicted by FIG. 45C with the outer lumen or sheath of the biopsy needle moving to cover the sample tray and enclose a tissue sample. In some embodiments, catheter begins to move around the biopsy needle. In some embodiments, a fourth step is depicted by FIG. 45D with the patch material being dispensed into the biopsy site. In some embodiments, a fifth step is depicted by FIG. 45E with the patch material being dispensed into the biopsy site and the catheter being left in the biopsy site as the biopsy needle and sample are removed from the biopsy site. In some embodiments, a sixth step is depicted by FIG. 45F with the needle and catheter fully removed from the biopsy site.

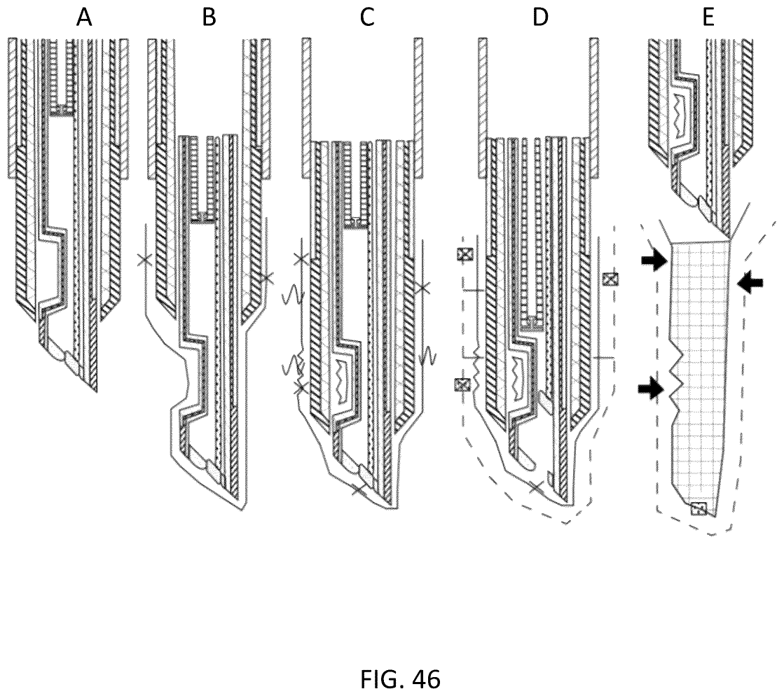

FIGS. 46A-46F depict a biopsy and subsequent repair of the biopsy site using a biopsy needle configured for cautery.

In some embodiments, a first step is depicted by FIG. 46A, wherein a biopsy needle is beginning insertion into the biopsy site. In some embodiments, a second step is depicted by FIG. 46B with the inner needle of the biopsy needle, which comprises a sample tray, being fully inserted into the biopsy site. In some embodiments, cauterization begins as the sheath enters the biopsy site. In some embodiments, a third step is depicted by FIG. 46C with the outer lumen or sheath of the biopsy needle moving to cover the sample tray and enclose a tissue sample. In some embodiments, cauterization continues. In some embodiments, a fourth step is depicted by FIG. 46D with cauterization being maintained. In some embodiments, a fifth step is depicted by FIG. 46E with the procedure ends as the biopsy needle and sample are removed from the biopsy site.

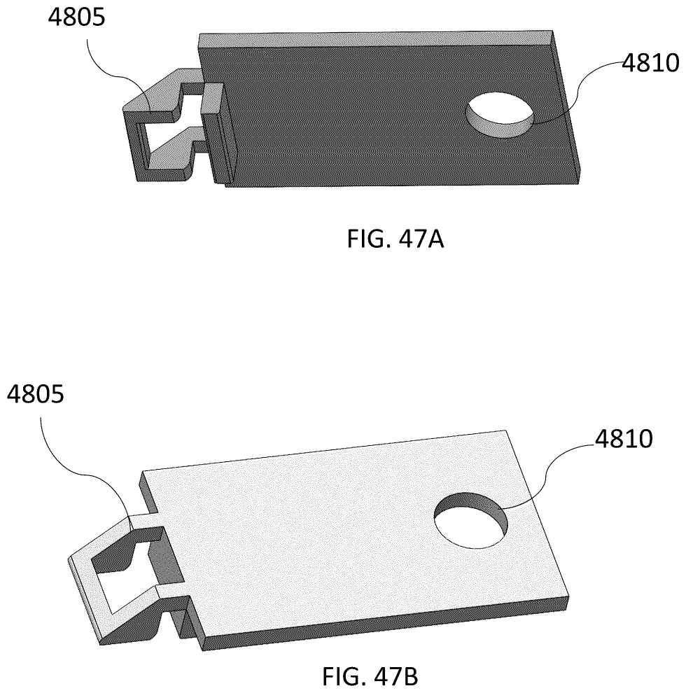

FIGS. 47A-47F depict a biopsy and subsequent repair of the biopsy site using a biopsy needle configured for cautery and dispensing a biocompatible patch material.

In some embodiments, a first step is depicted by FIG. 47A, wherein a biopsy needle is beginning insertion into the biopsy site. In some embodiments, a second step is depicted by FIG. 47B with the inner needle of the biopsy needle, which comprises a sample tray, being fully inserted into the biopsy site. In some embodiments, cauterization begins as the sheath enters the biopsy site. In some embodiments, a third step is depicted by FIG. 47C with the outer lumen or sheath of the biopsy needle moving to cover the sample tray and enclose a tissue sample. In some embodiments, cauterization continues as the waveguide moves to the distal tip of the needle. In some embodiments, a fourth step is depicted by FIG. 47D with cauterization being maintained and patch material being dispensed as a waveguide is retracted into the tip of the needle while still emitting light to initiate the patch material. In some embodiments, a fifth step is depicted by FIG. 47E with the procedure ends as the biopsy needle and sample are removed from the biopsy site. FIGS. 47A-47F depict a biopsy and subsequent repair of the biopsy site using a biopsy needle configured for cautery and dispensing a biocompatible patch material.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for delivering a biocompatible patch material to a biological site, the device comprising:
a housing comprising:
a cocking mechanism;
an activation button to release the cocking mechanism; and
a needle cartridge recess; and
a needle cartridge removably received by the needle cartridge recess of the housing, the needle cartridge comprising:
a storage cell containing the biocompatible patch material; and
a needle comprising a lumen in fluid communication with the storage cell;
wherein the needle cartridge is mechanically coupled to the cocking mechanism when the needle cartridge is received by the needle cartridge recess such that engagement of the activation button dispenses the biocompatible patch material from a distal end of the needle.

2. The device of claim 1, wherein the needle is a biopsy needle.

3. The device of claim 2, wherein engagement of the activation button activates the biopsy needle causing a sheath of the biopsy needle to cover a sample tray of the biopsy needle, thereby capturing a tissue sample from the biological site.

4. The device of claim 3, wherein activation of the biopsy need occurs prior to dispensing the biocompatible patch material from the distal end of the needle.

5. The device of claim 1, wherein the cocking mechanism comprises a cocking plate, wherein activation of the cocking mechanism rotates the cocking plate against a bias of a spring and locks the cocking plate in a first position.

6. The device of claim 5, wherein engagement of the activation button releases the cocking plate such that it moves to a second position, wherein the sample tray of the biopsy needle is not covered by the sheath when the cocking plate is in the second position.

7. The device of claim 6, wherein a second engagement of the activation button releases the cocking plate such that it moves to a third position, wherein the sheath covers the sample tray of the biopsy needle when the cocking plate is in the third position.

8. The device of claim 7, wherein a third engagement of the activation button releases the cocking plate such that it moves to a fourth position, wherein the biocompatible patch material is dispensed from the distal end of the needle when the cocking plate is in the fourth position.

9. The device of claim 8, further comprising a light source, wherein the light source emits light when the cocking plate is in the fourth position.

10. The device of claim 9, further comprising a waveguide to transmit light from the light source to the distal end of the needle.

11. The device of claim 10, wherein light from the light source is blocked from entering the waveguide until the cocking plate is in the fourth position.

12. The device of claim 1, further comprising a light source.

13. The device of claim 12, further comprising a waveguide to transmit light from the light source to the distal end of the needle.

14. The device of claim 12, wherein the housing comprises a cavity to receive the light source.

15. The device of claim 14, wherein the light source is removably received by the housing.

16. The device of any one of claims 12 to 15, wherein the biocompatible patch material comprises a photoinitiator, and wherein engagement of the activation button activates the biopsy needle to direct photons to activate the biocompatible patch material.

17. The device of claim 1, wherein engagement of the activation button extends at least a portion of the needle from the housing and engages releases the cocking mechanism such that the biocompatible patch material is dispensed through the retractable needle.

18. The device of claim 1, wherein the needle is configured for cauterization of the biological site.

19. The device of claim 18, wherein the needle cauterizes the biological site using thermal cauterization, RF cauterization, ultrasound cauterization, chemical cauterization, electrical cauterization, freeze cauterization, or a combination thereof.

20. The device of claim 1, wherein a thermal source is provided proximal or adjacent to the lumen or the storage cell, such that the biocompatible patch material is heated to reduce viscosity.

21. The device of claim 20, wherein the biocompatible patch material is heated to reduce the viscosity from greater than about 1000 cp to less than 500 cp.

22. A device comprising:
a needle comprising a lumen, wherein the lumen comprises an outlet;
a light emitter provided on a surface or disposed within the lumen of the needle; and
a fluid circuit connecting a fluid reservoir to the outlet of the lumen.

23. The device of claim 22, wherein the reservoir retains a biocompatible repair material.

24. The device of claim 23, wherein the biocompatible repair material comprises a photoinitiator.

25. The device of claim 22, wherein the needle further comprises a sample tray and a sheath.

26. The device of claim 25, wherein the sheath translates along the axis of the needle to enclose the sample tray.

27. The device of claim 23, wherein the needle further comprises a sheath, and wherein the sheath translates in a proximal direction along the axis of the needle to expose an opening to the reservoir.

28. The device of claim 27, wherein the needle further comprises a sample tray, and wherein the sheath translates in a distal direction along the axis of the needle to enclose the sample tray.

29. The device of claim 28, wherein the sheath translation of the sheath along the axis of the needle facilitates removal of the tissue sample from a tissue.

30. The device of claim 29, further comprising a syringe having a body and a plunger, wherein depression of the plunger toward a of the device causes: the biocompatible repair material to be expelled from the outlet; activation of the light emitter, and translation of the sheath in the distal direction to remove the tissue sample for the tissue and enclose the sample tray.

31. The device of claim 23, further comprising a syringe having a body and a plunger, wherein depression of the plunger toward a of the device causes: the biocompatible repair material to be expelled from the outlet; and activation of the light emitter.

32. The device of claim 22, wherein the needle is configured as a cauterizing needle.

33. The device of claim 32, wherein the cauterizing needle cauterizes the biological site using thermal cauterization, RF cauterization, ultrasound cauterization, chemical cauterization, electrical cauterization, freeze cauterization, or a combination thereof.

34. The device of claim 24, wherein a portion of the biocompatible repair material is activated within the lumen.

35. The device of claim 23, wherein the biocompatible repair material comprises a dual network material.

36. The device of claim 23, wherein the biocompatible repair material is provided in a cartridge.

37. The device of claim 36, wherein the cartridge is received by the device, and wherein receiving the cartridge provides the biocompatible repair material in fluid communication with the fluid circuit.

38. The device of claim 22, further comprising a thermal source.

39. The device of claim 38, wherein the thermal source transmits heat to a portion of a biocompatible patch material within the fluid circuit.

40. The device of claim 25, wherein the sheath rotates about the axis of the needle to enclose the sample tray.

41. A method for dispensing a photopolymerizable patch material comprising:
providing a tissue repair device comprising:
a needle comprising a lumen, wherein the lumen comprises a fluid outlet;
a light emitter disposed within the lumen of the needle; and
a fluid circuit connecting a fluid reservoir to the outlet of the lumen;
providing the photopolymerizable patch material to the fluid reservoir;
actuating the fluid circuit to dispense the photopolymerizable patch material from the outlet.

42. The method of claim 41, further comprising activating the light emitter to activate the photopolymerizable patch material after the patch material is dispensed from the outlet.

43. The method of claim 41, further comprising activating the light emitter to photopolymerize a portion of the photopolymerizable patch material in the lumen.

44. The method of claim 41, further comprising activating the light emitter to photopolymerize a portion of the photopolymerizable patch material in the lumen.

45. The method of claim 41, further comprising inflating a balloon to compress the photopolymerizable patch material.

46. The method of claim 45, wherein the photopolymerizable patch material is compressed by the balloon after it has been dispensed from the outlet.

47. The method of claim 46, wherein the photopolymerizable patch material is compressed by the balloon against a surface requiring repair.

48. The method of claim 47, wherein the surface is a biological surface.

49. The method of claim 48, biological surface is a biopsy site.

50. A method of collecting a tissue sample and repairing a wound created during collection of the tissue sample, the method comprising:
providing a biopsy needle having a sample tray and a sheath at a biological site;
sliding the sheath over the sample tray, thereby cutting the tissue sample from the biological site and retaining the tissue sample within the biopsy needle;
dispensing a biocompatible adhesive into the wound; and
retracting the needle from the biological site.

51. The method of claim 50, further comprising cauterizing wound prior to dispensing the biocompatible adhesive.

52. The method of claim 50, wherein the biocompatible adhesive is dispensed from a distal end of the biopsy needle.

53. The method of claim 52, wherein the biopsy needle comprises a lumen, wherein the lumen comprises a fluid outlet for dispensing the biocompatible adhesive into the wound.

54. The method of claim 53, wherein the fluid outlet is distal to the sample tray.

55. The method of claim 52, wherein the sheath comprises an electrode, and wherein the method further comprises actuating the electrode to cauterize the wound after cutting the tissue sample from the biological site.

56. The method of claim 55, wherein cauterizing the would is prior to dispensing the biocompatible adhesive.

57. The method of claim 56, wherein the biocompatible adhesive comprises a photoinitiator, and wherein the biopsy needle further comprises a wave guide to direct light from a light source to the biocompatible adhesive.

58. The method of claim 57, wherein the biocompatible adhesive comprises a photoinitiator, and wherein the biopsy needle further comprises a wave guide to direct light from a light source to the biocompatible adhesive.

59. The method of claim 55, wherein the waveguide is provided within the biopsy needle, and wherein an end of the wave guide is distal to the sample tray.

60. A device for collection of a tissue sample and repair of a biological site, the biological being located within a body of a subject, wherein said device is configured such that removal of the device from within the body of the subject is not required between collecting the tissue sample and repairing the biological site.
